(19) United States
(12) Patent Application Publication
(10) Pub. No.: US 2026/0250375 A1
(43) Pub. Date: Aug. 27, 2026

(54) COMPOUND TARGETING IL-23A AND TNF-ALPHA AND USES THEREOF

(71) Applicants: BOEHRINGER INGELHEIM INTERNATIONAL GMBH, INGELHEIM AM RHEIN (DK); MACROGENICS, INC., ROCKVILLE, MD (US)

(72) Inventors: Rachel Rebecca Barrett, Bethel, CT (US); Leslie S. Johnson, Darnestown, MD (US); Sanjaya Singh, Sandy Hook, CT (US); Kathleen Last-Barney, Ridgefield, CT (US); Daw-Tsun Shih, Ridgefield, CT (US); Patricia Giblin, Ridgefield, CT (US); Scott Brodeur, Ridgefield, CT (US); Nelamangala Nagaraja, Ridgefield, CT (US)

(21) Appl. No.: 19/547,752

(22) Filed: Feb. 24, 2026

Related U.S. Application Data
(63) Continuation of application No. 18/178,783, filed on Mar. 6, 2023.
Application No. 18/178,783 is a division of application No. 17/002,890, filed on Aug. 26, 2020, now Pat. No. 11,680,096.
Application No. 17/002,890 is a division of application No. 16/052,133, filed on Aug. 1, 2018, now Pat. No. 10,793,629.
Application No. 16/052,133 is a division of application No. 14/844,338, filed on Sep. 3, 2015, now Pat. No. 10,059,763.
(60) Provisional application No. 62/045,498, filed on Sep. 3, 2014.

(51) Int. Cl. C07K 16/24; A61K 39/00; A61K 39/395; C12N 5/07; C12N 5/0783; C12N 15/63; C12P 15/00; G01N 33/575

(52) U.S. Cl. CPC C07K 16/244; C07K 16/241; C12N 5/0638; A61K 39/3955; A61K 2039/505; C07K 2317/31; C07K 2317/76; C07K 2317/92; C07K 2317/94; C12N 5/06; C12N 15/63; C12P 15/00; G01N 33/575

(57) ABSTRACT

The disclosure relates to compounds specific for IL23A and TNF-alpha, compositions comprising the compounds, and methods of use thereof. Nucleic acids, cells, and methods of production related to the compounds and compositions are also disclosed.

### CROSS-REFERENCE TO RELATED APPLICATIONS

This application is a continuation of U.S. application Ser. No. 18/178,783, filed Mar. 6, 2023, which is a divisional of U.S. application Ser. No. 17/002,890, filed Aug. 26, 2020, now U.S. Pat. No. 11,680,096, which is a divisional of U.S. application Ser. No. 16/052,133, filed Aug. 1, 2018, now U.S. Pat. No. 10,793,629, which is a divisional of U.S. application Ser. No. 14/844,338, filed Sep. 3, 2015, now U.S. Pat. No. 10,059,763, which claims priority to Provisional Appl. No. 62/045,498, filed Sep. 3, 2014, the disclosures of each of which are hereby incorporated by reference in their entireties.

### REFERENCE TO AN ELECTRONIC SEQUENCE LISTING

The contents of the electronic sequence listing (1157443o001005.xml; Size: 262,870 bytes; and Date of Creation: Feb. 11, 2026) is herein incorporated by reference in its entirety.

### BACKGROUND OF THE INVENTION

Inflammation involves an innate and adaptive immune response that is required for fighting infection. However, when the inflammation becomes unchecked autoimmune or autoinflammatory diseases, neurodegenerative disease, and even cancer can develop. It is well established that inhibiting activity of proinflammatory cytokines such as IL1, TNF-alpha, IL6, IL12, IL17, IL18, or IL23 reduces inflammation and suppresses specific pathways that activate immune cells.

Interleukin 23 (IL23) is a heterodimeric cytokine consisting of two subunits, p40 and p19. The p19 subunit is also referred to as IL-23A. While the p19 subunit is unique to IL23, the p40 subunit is shared with the cytokine IL12. IL23 is emerging as a key regulator of pathogenic Th17, γδ T and innate lymphoid cells (ILCs) driving the production of IL17, IL22 and other cytokines that lead to local tissue inflammation and damage. IL23 promotes upregulation of the matrix metalloprotease MMP9, increases angiogenesis, reduces CD8+ T cell infiltration, and has been implicated in the development of cancerous tumors. In addition, in conjunction with IL6 and TGF-beta1, IL23 stimulates naive CD4+ T cells to differentiate into Th17 cells. In turn, the Th17 cells produce IL17, a proinflammatory cytokine that enhances T cell priming and stimulates the production of proinflammatory cytokines such as IL1, IL6, TNF-alpha, NOS-2, and also induces expression of chemokines resulting in inflammation and disease pathogenesis. IL23 exerts its effects via a cell surface receptor composed of the IL12β1 subunit of IL12 receptor partnered with a unique IL23R subunit. Expression of the IL23R is restricted to specific populations of immune cells and is found primarily on subsets of T cells (αβ and γδ TCR+) and NK cells.

In mice, genetic ablation of the IL23p19 gene results in selective loss of IL23 function accompanied by severely compromised T-dependent immune responses, including reduced production of antigen-specific immunoglobulins and impaired delayed type hypersensitivity responses (Ghilardi N, et al. (2004) J. Immunol. 172(5): 2827-33). Knockout mice deficient in either IL23p40 or IL23p19, or in either subunit of the IL23 receptor (IL23R and IL12-beta1), develop less severe symptoms in animal models of multiple sclerosis, arthritis and inflammatory bowel disease. Similar results have been obtained using an antibody specific for IL23p19 in EAE and a T cell mediated colitis model further substantiates the role of IL23 in these disease settings (Chen Y. et al. (2006) J. Clin. Invet. 116(5):1317-26; Elson C O. et al. (2007) Gastroenterology 132(7): 2359-70). This highlights the importance of IL23 in chronic inflammation (Langowski et al. (2006) Nature 442 (7101): 461-5; Kikly K, et al. (2006) Curr. Opin. Immunol. 18 (6): 670-5). In addition, elevated IL23 production has been implicated as being a major factor in inflammatory arthritis and in inflammatory autoimmune diseases (Adamopoulos et al. (2011) J. Immunol. 187: 593-594; and Langris et al. (2005) J. Exp. Med. 201:233-240). A connection between IL23, its downstream cytokine IL22, and bone formation has been published in a mouse model system in which IL23 is overexpressed (Sherlock et al. (2012) Nat. Med. 18: 1069-76).

The homotrimeric TNF-α cytokine is expressed predominantly by macrophages, lymphocytes, endothelial cells and fibroblasts and binds two distinct receptors: TNFRI, expressed on nearly all cell types and TNFRII, with more limited expression on immune cells (CD4+ T cells, NK cells). Like many TNF superfamily members, TNF-α exists as both membrane and soluble forms, the soluble form arising from cleavage of the membrane form by the ADAM12 metalloprotease (TACE, TNFα converting enzyme). Both membrane-bound and soluble forms of the cytokine are biologically active.

Tumor necrosis factor (TNF-alpha/TNF-α) is a proinflammatory cytokine that stimulates the acute phase of inflammation. Tumor necrosis factor increases vascular permeability through induction of IL8, thereby recruiting macrophage and neutrophils to a site of infection. Once present, activated macrophages continue to produce TNF-alpha, thereby maintaining and amplifying the inflammatory response. The primary role of TNF-alpha is the regulation of immune cells; however, TNF-alpha is also involved in the regulation of a wide spectrum of biological processes including cell proliferation, differentiation, apoptosis, lipid metabolism, and coagulation. TNF-alpha is able to induce inflammation, induce apoptotic cell death, inhibit tumorigenesis and inhibit viral replication.

Dysregulation of TNF-alpha production has been implicated in a variety of human diseases, including autoimmune disease (e.g. rheumatoid arthritis (RA), Crohn's disease, multiple sclerosis), inflammatory bowel disease (IBD), ulcerative colitis, psoriasis, toxic shock, graft versus host disease, insulin resistance, Alzheimer's disease, cancer, and major depression (Swardfager W, et al. (2010) Biol Psychiatry 68 (10): 930-941; (Locksley R M, et al. (2001) Cell 104 (4): 487-501; Dowlati et al., (2010) Biol Psychiatry 67 (5): 446-457; Brynskov J. et al. (2002) Gut 51 (1): 37-43).

Antibodies have been used as biologic therapies for inhibition of TNF-alpha and IL23 in order to treat a variety of inflammatory diseases. Infliximab (Centocor, Malvern, Pa.) described in U.S. Pat. Nos. 6,277,969, 6,284,471, and 6,790,444, is a chimeric anti-TNF-alpha monoclonal IgG antibody bearing human IgG4 constant and mouse variable regions and is used clinically to treat rheumatoid arthritis, psoriatic arthritis, ankylosing spondylitis, Crohn's disease, ulcerative colitis and plaque psoriasis. Monoclonal antibody adalimumab (clone D2E7; Abbott Laboratories, Abbott Park, Illinois) described in U.S. Pat. No. 6,090,382, is an anti-TNF-alpha therapy used clinically to treat rheumatoid arthritis, Crohn's disease, psoriasis, psoriatic arthritis, ankylosing spondylitis, and juvenile idiopathic arthritis. Golimumab is a TNF-alpha blocker used to treat rheumatoid arthritis, psoriatic arthritis, ankylosing spondylitis, and ulcerative colitis. In addition, human monoclonal antibody ustekinumab (Centocor, Inc, Malvern, Pa.), described in U.S. Pat. Nos. 6,902,734 and 7,166,285, is directed against interleukin 12 and interleukin 23 (specifically the p40 subunit), is clinically used to treat severe plaque psoriasis, and is further being investigated for the treatment of psoriatic arthritis, multiple sclerosis, and sarcoidosis. However, anti-TNF-α therapies have reported side effects [see for example: Keane J et al. (2001)]. Tuberculosis is associated with infliximab, a tumor necrosis factor α-neutralizing agent. N Engl J Med 345 (15):1098-1104; Scheinfeld N. (2005) Adalimumab: a review of side effects. Expert Opin Drug Saf. 4(4):637-41; Chovel-Sella A et al. (2012) Clinical efficacy and adverse effects of golimumab in the treatment of rheumatoid arthritis. Isr Med Assoc J. 14(6):390-4]. Identification of more efficacious treatments should allow for administration of reduced dosages, as well as lower costs associated with the treatment.

There remains a need for compositions with increased efficacy for treating and preventing autoimmune or inflammatory diseases.

### SUMMARY OF THE INVENTION

Provided herein are compounds specific for TNF-alpha and IL23A, compositions comprising such compounds, as well as methods of use and production thereof.

Aspects of the disclosure relate to a compound comprising a first polypeptide and a second polypeptide, wherein:
(A) said first polypeptide comprises:
   (i) a light chain variable domain of a first immunoglobulin (VL1) specific for a first target protein;
   (ii) a heavy chain variable domain of a second immunoglobulin (VH2) specific for a second target protein; and
   (iii) a hinge region, a heavy chain constant region 2 (CH2) and a heavy chain constant region 3 (CH3); and
(B) said second polypeptide comprises:
   (i) a light chain variable domain of the second immunoglobulin (VL2) specific for said second target protein;
   (ii) a heavy chain variable domain of the first immunoglobulin (VH1) specific for said first target protein;
wherein:
   (i) said VL1 and VH1 associate to form a binding site that binds said first target protein;
   (ii) said VL2 and VH2 associate to form a binding site that binds said second target protein;
   (iii) said heavy chain constant region 2 (CH2) comprises a tyrosine at position 252, a threonine at position 254 and a glutamic acid a position 256, numbered according to the EU index as in Kabat for a conventional antibody; and
   (iv) said first target protein is TNF-alpha and said second target protein is IL-23A or said first target protein is IL-23A and said second target protein is TNF-alpha,
wherein:
   (i) said VL1 comprises SEQ ID NO:2, said VH1 comprises SEQ ID NO:1, said VL2 comprises SEQ ID NO:8 and said VH2 comprises SEQ ID NO:7; or
   (ii) said VL1 comprises SEQ ID NO:4 or 6, said VH1 comprises SEQ ID NO:3 or 5, said VL2 comprises SEQ ID NO:8 and said VH2 comprises SEQ ID NO:7; or
   (iii) said VL1 comprises SEQ ID NO:8, said VH1 comprises SEQ ID NO:7, said VL2 comprises SEQ ID NO:2 and said VH2 comprises SEQ ID NO:1; or
   (iv) said VL1 comprises SEQ ID NO:8, said VH1 comprises SEQ ID NO:7, said VL2 comprises SEQ ID NO:4 or 6 and said VH2 comprises SEQ ID NO:3 or 5.

In some embodiments, in (ii) said VL1 comprises SEQ ID NO:4, said VH1 comprises SEQ ID NO:3, said VL2 comprises SEQ ID NO:8 and said VH2 comprises SEQ ID NO:7. In some embodiments, in (ii) said VL1 comprises SEQ ID NO:6, said VH1 comprises SEQ ID NO:5, said VL2 comprises SEQ ID NO:8 and said VH2 comprises SEQ ID NO:7. In some embodiments, in (iv) said VL2 comprises SEQ ID NO:4, said VH2 comprises SEQ ID NO:3, said VL1 comprises SEQ ID NO:8 and said VH1 comprises SEQ ID NO:7. In some embodiments, in (iv) said VL2 comprises SEQ ID NO:6, said VH2 comprises SEQ ID NO:5, said VL1 comprises SEQ ID NO:8 and said VH1 comprises SEQ ID NO:7.

In some embodiments, said first polypeptide further comprises a first linker between said VL1 and said VH2 and said second polypeptide further comprises a second linker between said VL2 and said VH1. In some embodiments, said first linker or said second linker comprises the amino acid sequence GGGSGGG (SEQ ID NO:9). In some embodiments, said first linker and said second linker comprise the amino acid sequence GGGSGGG (SEQ ID NO:9).

In some embodiments, said first polypeptide further comprises a heavy chain constant region 1 domain (CH1) and said second polypeptide further comprises a light chain constant region domain (CL), wherein said CL and said CH1 are associated together via a disulfide bond to form a C1 domain.

In some embodiments, said first polypeptide further comprises a third linker between said VH2 and said CH1 and said second polypeptide further comprises a fourth linker between said VH1 and said CL. In some embodiments, said third linker comprises the amino acid sequence FNRGES (SEQ ID NO:11). In some embodiments, said fourth linker comprises the amino acid sequence VEPKSS (SEQ ID NO:12). In some embodiments, said third linker comprises the amino acid sequence FNRGES (SEQ ID NO:11) and said fourth linker comprises the amino acid sequence VEPKSS (SEQ ID NO:12). In some embodiments, third linker or said fourth linker comprises the amino acid sequence LGGGSG (SEQ ID NO:10). In some embodiments, said third linker and said fourth linker comprise the amino acid sequence LGGGSG (SEQ ID NO:10).

In some embodiments, said heavy chain constant region 2 (CH2) comprises an alanine at positions 234 and an alanine at position 235, numbered according to the EU index as in Kabat for a conventional antibody.

In some embodiments, the amino acid sequence of said hinge region, said heavy chain constant region 2 (CH2) or said heavy chain constant region 3 (CH3) is derived from a IgG1 or from a IgG4. In some embodiments, said hinge region comprises the amino acid sequence EPKSCDKTHTCPPCP (SEQ ID NO:40).

In some embodiments, said compound comprises two said first polypeptides and two said second polypeptides, wherein said two first polypeptides are associated together via at least one disulfide bond. In some embodiments, said compound comprises two said first polypeptides and two said second polypeptides, wherein said two first polypeptides are associated together via at least one disulfide bond and wherein each of said first polypeptide is associate to one said second polypeptide via at least one disulfide bond.

In some embodiments,
(i) said first polypeptide comprises the amino acid sequence of SEQ ID NO:13 and said second polypeptide comprises the amino acid sequence of SEQ ID NO:14;
(ii) said first polypeptide comprises the amino acid sequence of SEQ ID NO:15 and said second polypeptide comprises the amino acid sequence of SEQ ID NO:16;
(iii) said first polypeptide comprises the amino acid sequence of SEQ ID NO:17 and said second polypeptide comprises the amino acid sequence of SEQ ID NO:18;
(iv) said first polypeptide comprises the amino acid sequence of SEQ ID NO:19 and said second polypeptide comprises the amino acid sequence of SEQ ID NO:20;
(v) said first polypeptide comprises the amino acid sequence of SEQ ID NO:21 and said second polypeptide comprises the amino acid sequence of SEQ ID NO:22;
(vi) said first polypeptide comprises the amino acid sequence of SEQ ID NO:23 and said second polypeptide comprises the amino acid sequence of SEQ ID NO:24;
(vii) said first polypeptide comprises the amino acid sequence of SEQ ID NO:25 and said second polypeptide comprises the amino acid sequence of SEQ ID NO:26;
(viii) said first polypeptide comprises the amino acid sequence of SEQ ID NO:27 and said second polypeptide comprises the amino acid sequence of SEQ ID NO:28;
(ix) said first polypeptide comprises the amino acid sequence of SEQ ID NO:29 and said second polypeptide comprises the amino acid sequence of SEQ ID NO:30;
(x) said first polypeptide comprises the amino acid sequence of SEQ ID NO:31 and said second polypeptide comprises the amino acid sequence of SEQ ID NO:32;
(xi) said first polypeptide comprises the amino acid sequence of SEQ ID NO:33 and said second polypeptide comprises the amino acid sequence of SEQ ID NO:34; or
(xi) said first polypeptide comprises the amino acid sequence of SEQ ID NO:35 and said second polypeptide comprises the amino acid sequence of SEQ ID NO:36.

In some embodiments, wherein said compound comprises two said first polypeptides and two said second polypeptides, wherein said two first polypeptides are associated together via at least one disulfide bond.

In some embodiments, said compound comprises two said first polypeptides and two said second polypeptides, and wherein the CH2 and CH3, and CH1 if present, of one of the first polypeptides associates with the CH2 and CH3, and CH1 if present, of the other of the first polypeptides to form a tetravalent molecule. In some embodiments, said compound comprises two said first polypeptides and two said second polypeptides, wherein each of said first polypeptides comprises a CH1, a CH2 and a CH3 and each of said second polypeptides comprises a CL and wherein the CH2 and CH3 of one of the first polypeptides associates with the CH2 and CH3 of the other of the first polypeptides and the CH1 of each said first polypeptides associates with the CL of one said second polypeptides to form a tetravalent molecule.

Other aspects of the disclosure relate to a first compound that competes with a second compound for binding to IL-23A and to TNF-alpha, wherein said first compound comprises a third polypeptide and fourth polypeptide, wherein:
(A) said third polypeptide comprises:
   (i) a light chain variable domain of a first immunoglobulin (VL1) specific for a first target protein;
   (ii) a heavy chain variable domain of a second immunoglobulin (VH2) specific for a second target protein; and
   (iii) a hinge region, a heavy chain constant region 2 (CH2) and a heavy chain constant region 3 (CH3); and
(B) said fourth polypeptide comprises:
   (i) a light chain variable domain of the second immunoglobulin (VL2) specific for said second target protein;
   (ii) a heavy chain variable domain of the first immunoglobulin (VH1) specific for said first target protein;
and wherein
   (i) said VL1 and VH1 associate to form a binding site that binds said first target protein;
   (ii) said VL2 and VH2 associate to form a binding site that binds said second target protein; and
   (iii) said first target protein is TNF-alpha and said second target protein is IL-23A or said first target protein is IL-23A and said second target protein is TNF-alpha,
and wherein said second compound comprises a first polypeptide and a second polypeptide, wherein:
   (i) said first polypeptide comprises the amino acid sequence of SEQ ID NO:13 and said second polypeptide comprises the amino acid sequence of SEQ ID NO:14;
   (ii) said first polypeptide comprises the amino acid sequence of SEQ ID NO:15 and said second polypeptide comprises the amino acid sequence of SEQ ID NO:16;
   (iii) said first polypeptide comprises the amino acid sequence of SEQ ID NO:17 and said second polypeptide comprises the amino acid sequence of SEQ ID NO:18;
   (iv) said first polypeptide comprises the amino acid sequence of SEQ ID NO:19 and said second polypeptide comprises the amino acid sequence of SEQ ID NO:20;
   (v) said first polypeptide comprises the amino acid sequence of SEQ ID NO:21 and said second polypeptide comprises the amino acid sequence of SEQ ID NO:22;
   (vi) said first polypeptide comprises the amino acid sequence of SEQ ID NO:23 and said second polypeptide comprises the amino acid sequence of SEQ ID NO:24;
   (vii) said first polypeptide comprises the amino acid sequence of SEQ ID NO:25 and said second polypeptide comprises the amino acid sequence of SEQ ID NO:26;
   (viii) said first polypeptide comprises the amino acid sequence of SEQ ID NO:27 and said second polypeptide comprises the amino acid sequence of SEQ ID NO:28;
   (ix) said first polypeptide comprises the amino acid sequence of SEQ ID NO:29 and said second polypeptide comprises the amino acid sequence of SEQ ID NO:30;
   (x) said first polypeptide comprises the amino acid sequence of SEQ ID NO:31 and said second polypeptide comprises the amino acid sequence of SEQ ID NO:32;
   (xi) said first polypeptide comprises the amino acid sequence of SEQ ID NO:33 and said second polypeptide comprises the amino acid sequence of SEQ ID NO:34; or
   (xii) said first polypeptide comprises the amino acid sequence of SEQ ID NO:35 and said second polypeptide comprises the amino acid sequence of SEQ ID NO:36.

Yet other aspects of the disclosure relate to a pharmaceutical composition comprising a compound described herein, such as a compound described above.

Other aspects of the disclosure relate to a method of treating an autoimmune or an inflammatory disease comprising administering a compound described herein, such as a compound described above, or a pharmaceutical composition comprising said compound to a subject.

Yet other aspects of the disclosure relate to a compound described herein, such as a compound described above, for use in medicine. In some embodiments, said use is the treatment of an autoimmune or an inflammatory disease.

Other aspects of the disclosure relate to a pharmaceutical composition comprising a compound described herein, such as a compound described above, for use in medicine. In some embodiments, said use is the treatment of an autoimmune or an inflammatory disease.

Yet other aspects of the disclosure relate to a use of a compound described herein, such as a compound described above, in the manufacture of a medicament for use in medicine. In some embodiments, said use is the treatment of an autoimmune or an inflammatory disease.

Other aspects of the disclosure relate to a use of a pharmaceutical composition described herein, such as a pharmaceutical composition described above, in the manufacture of a medicament for use in medicine. In some embodiments, said use is the treatment of an autoimmune or an inflammatory disease.

Yet other aspects of the disclosure relate to a nucleic acid comprising a nucleotide sequence encoding a polypeptide described herein, such as a polypeptide described above.

Other aspects of the disclosure relate to a vector comprising said nucleic acid. In some embodiments, the vector comprises a promoter operably linked to said nucleic acid. Other aspects of the disclosure relate to a cell comprising said nucleic acid or said vector.

Other aspects of the disclosure relate to a method of producing a compound or polypeptide as described herein, such as a polypeptide described above, comprising obtaining a cell described herein, such a cell described above, and expressing a nucleic acid as described herein in said cell. In some embodiments, the method further comprises isolating and purifying said polypeptide or compound.

The details of one or more embodiments of the disclosure are set forth in the description below. Other features or advantages of the present disclosure will be apparent from the following drawings and detailed description of several embodiments, and also from the appending claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1A is a diagram of an exemplary compound specific for TNF-alpha and IL23A. The first polypeptide chain contains CH3, CH2, VH₂(VH_{II}) and VL₁ (VL_{I}) domains. The second polypeptide chain contains VH₁ (VH_{I}) and VL₂ (VL_{II}) domains. VL₁ and VH₁ are specific for a first target protein (either TNF-alpha or IL23A) and VL₂ and VH₂ are specific for a second target protein (either IL23A or TNF-alpha). The upper panel shows each polypeptide chain separately. The lower panel shows a tetravalent compound formed through association of the CH2 and CH3 domains of one first polypeptide with the CH2 and CH3 domains of another first polypeptide. The binding domains for the first and second target protein are formed through association of VH₁ and VL₁ and through association VH₂ and VL₂, respectively.

FIG. 1B is a diagram of another exemplary compound specific for TNF-alpha and IL23A. The first polypeptide chain contains CH3, CH2, CH1, VH₂ (VH_{II}) and VL₁ (VL_{I}) domains. The second polypeptide chain contains CL, VH₁ (VH_{I}) and VL₂ (VL_{II}) domains. VL₁ and VH₁ are specific for a first target protein (either TNF-alpha or IL23A) and VL₂ and VH₂ are specific for a second target protein (either IL23A or TNF-alpha). The upper panel shows each polypeptide chain separately. The lower panel shows a tetravalent compound formed through association of the CH2 and CH3 domains of one first polypeptide with the CH2 and CH3 domains of another first polypeptide. The binding domains for the first and second target protein are formed through association of VH₁ and VL₁ and through association VH₂ and VL₂, respectively. The compound is further associated through interactions between the CL and CH1 domains.

FIG. 2 is a graph showing the serum concentrations of Compound M and its YTE mutant Compound A in male cynomolgus monkey (mean±SD; N=3) after 1 mg/kg IV 10 minute infusion.

FIG. 3 is a graph show serum concentrations of Compound 0 and its corresponding YTE mutant Compound E in male cynomolgus monkey (mean±SD; N=3) after 1 mg/kg IV 10 minute infusion.

FIG. 4 is a series of graphs and a table showing that compound E maintained functional potency vs. IL23 in vivo. Mice were dosed equimolar with either control antibody 3 (IL23A monoclonal antibody) or compound E and challenged with human IL23 twice to induce ear inflammation. Twenty four hours after the final injection, ears were collected and analyzed for mouse IL17A and mouse IL22 as a measure of functional blockade of IL23. Compound E maintained functional potency in vivo vs. control antibody 3 (IL23A monoclonal antibody) based on terminal exposure and level of efficacy. Control antibody 3: open squares, triangles and diamonds. Compound E: full squares, triangles and diamonds. MW: molecular weight.

FIG. 5 is a series of graphs and a table showing that Compound E maintained functional potency vs. TNF in vivo. Mice were dosed equimolar with either control antibody 2 (TNFa monoclonal antibody) or compound E and challenged with human TNF. Two hours after the challenge, whole blood was collected and serum analyzed for mouse KC and mouse IL-6 as a measure of functional blockade of TNF. Compound E maintained functional potency in vivo vs. anti-TNF based on terminal exposure and level of efficacy. Control antibody 3: open squares, triangles and diamonds. Compound E: full squares, triangles and diamonds. MW: molecular weight.

### DETAILED DESCRIPTION OF THE INVENTION

Described herein compounds that bind to both TNF-alpha (also referred to herein as TNF-α or TNFa) and IL23A (also referred to as IL23p19 or IL-23A). To date, there have been no approved compounds that target both TNF-alpha and IL23A. There are limited studies with simultaneous neutralization of two/more key inflammatory mediators using bio-therapeutics approach. While these studies failed to show improvement in clinical outcomes that were measured for rheumatoid arthritis (RA), a bi-functional therapeutic targeting the same combination has not been described to date. In addition, such combinations may increase side effects, such as the risk of infection (see, e.g., Genovese, M. C., Cohen, S., Moreland, L., Lium, D., Robbins, S., et al. (2004). Arth. Rheum. 50, 1412-9; Genovese, M. C., Cohen, S., Moreland, L., Lium, D., Robbins, S., et al. (2004). Arth. Rheum. 50, 1412-9; and Weinblatt, M., Schiff, M., Goldman, A. Kremer, J., Luggen, M., et al. (2007). Ann. Rheum. Dis. 66, 228-34). Further, such bi-specific compounds have been difficult to design, due to issues related to solubility (e.g., aggregation) and stability (e.g., poor pharmacokinetics).

Surprisingly, the compounds described herein that bind to both TNF-alpha and IL23A have been found to have similar or improved properties compared to individual antibodies that target either IL23A or TNF-alpha. These compounds were also found to have suitable pharmacokinetics and were soluble at suitable ranges for dosing purposes. Further, in some embodiments, there are advantages of single administration over multiple individual dose administration from the perspective of side effects of the individual therapies, and lower dosage. In addition, in some embodiments, the CMC properties of the compounds showed that compounds had low aggregation. In one aspect, exemplary compounds showed particularly low aggregation. It was also shown that the linkers were optimized to improve stability and prevented cleavage and that the YTE mutation improved Fc Rn affinity. The compounds described herein are believed to have one or more advantageous properties, e.g., decreased side effects, increased ease and safety of administration, an increased half-life, increased binding affinity, or increased inhibitory activity, compared to standard antibody molecules, e.g., an IgG molecule or antigen-binding fragment (Fab).

Accordingly, aspects of the disclosure relate to compounds specific for both TNF-alpha and IL23A, as well as methods of use and production of such compounds.

### Compounds

Aspects of the disclosure relate to a compound specific for both TNF-alpha and IL23A. An exemplary protein sequence for TNF-alpha and an exemplary protein sequence for IL23A are shown below.

>NP_000585.2-TNF-alpha [Homo sapiens]
(SEQ ID NO: 144)
MSTESMIRDVELAEEALPKKTGGPQGSRRCLFLSLFSFLIVAGATTL
FCLLHFGVIGPQREEFPRDLSLISPLAQAVRSSSRTPSDKPVAHVVA
NPQAEGQLQWLNRRANALLANGVELRDNQLVVPSEGLYLIYSQVLFK
GQGCPSTHVLLTHTISRIAVSYQTKVNLLSAIKSPCQRETPEGAEAK
PWYEPIYLGGVFQLEKGDRLSAEINRPDYLDFAESGQVYFGIIAL
>NP_057668.1-IL23A [Homo sapiens]
(SEQ ID NO: 145)
MLGSRAVMLLLLLPWTAQGRAVPGGSSPAWTQCQQLSQKLCTLAWSA
HPLVGHMDLREEGDEETTNDVPHIQCGDGCDPQGLRDNSQFCLQRIH
QGLIFYEKLLGSDIFTGEPSLLPDSPVGQLHASLLGLSQLLQPEGHH
WETQQIPSLSPSQPWQRLLLRFKILRSLQAFVAVAARVFAHGAATLS
P (amino acids 1-19 are a predicted signal
sequence)

In some embodiments, the compound comprises a first polypeptide and a second polypeptide. In some embodiments, the first polypeptide comprises (i) a light chain variable domain of a first immunoglobulin (VL1) specific for a first target protein, (ii) a heavy chain variable domain of a second immunoglobulin (VH2) specific for a second target protein; and (iii) a hinge region, a heavy chain constant region 2 (CH2) and a heavy chain constant region 3 (CH3). In some embodiments, the first polypeptide further comprises a heavy chain constant region 1 (CH1). In some embodiments, the second polypeptide comprises: (i) a light chain variable domain of the second immunoglobulin (VL2) specific for the second target protein; (ii) a heavy chain variable domain of the first immunoglobulin (VH1) specific for the first target protein. In some embodiments, the first polypeptide further comprises a light chain constant region (CL).

It is to be understood that the variable domains and constant domains/regions of the first polypeptide can be in any order and that the variable domains and constant domains/regions (if any) of the second polypeptide can be in any order. Multiple exemplary configurations for the domains/regions on the first and second polypeptide from N-terminus to C-terminus are shown below.
First polypeptide configuration 1: N-VL1-VH2-hinge-CH2-CH3-C
First polypeptide configuration 2: N-VH2-VL1-hinge-CH2-CH3-C
First polypeptide configuration 3: N-VL1-VH2-CH1-hinge-CH2-CH3-C
First polypeptide configuration 4: N-VH2-VL1-CH1-hinge-CH2-CH3-C
Second polypeptide configuration 1: N-VL2-VH1-C
Second polypeptide configuration 2: N-VH1-VL2-C
Second polypeptide configuration 3: N-VL2-VH1-CL-C
Second polypeptide configuration 4: N-VH1-VL2-CL-C

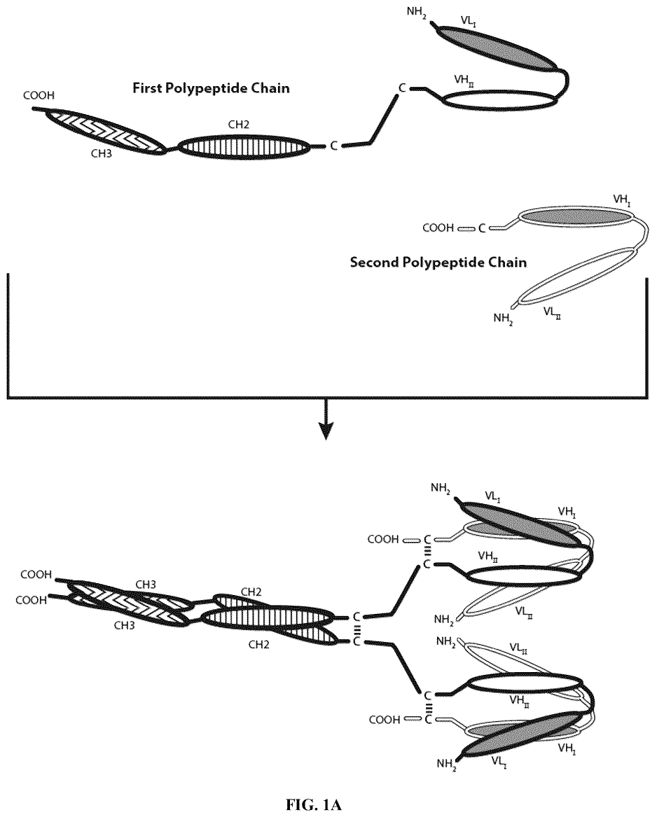

Exemplary configurations of the compound are shown in FIGS. 1A and 1B. In some embodiments, the compound comprises the first polypeptide in configuration 1 and the second polypeptide in configuration 1. In some embodiments, the compound comprises the first polypeptide in configuration 3 and the second polypeptide in configuration 3.

In some embodiments, the variable regions of the first polypeptide and the second polypeptide associate with one another to form a binding site for the first target protein and a binding site for the second target protein. In some embodiments, the VL1 of the first polypeptide and the VH1 of the second polypeptide associate to form a binding site that binds the first target protein and the VL2 of the second polypeptide and the VH2 of the first polypeptide associate to form a binding site that binds the second target protein. In some embodiments, the first target protein is TNF-alpha and the second target protein is IL23A. In other embodiments, the first target protein is IL23A and the second target protein is TNF-alpha. It is to be understood that the terms “first” and “second” are not meant to imply a level of importance to either target protein.

Exemplary combinations of sequences for each of VL1, VH1, VL2, and VH2 are provided below in Table 1 and also in Table 2A in Example 1.

TABLE 1
Exemplary combinations of sequences for VL1, VH1, VL2, and VH2.

| Combination | | | | |
|---|---|---|---|---|
| Number | VL1 sequence | VH1 sequence | VL2 sequence | VH2 sequence |
| 1 | SEQ ID NO: 2 | SEQ ID NO: 1 | SEQ ID NO: 8 | SEQ ID NO: 7 |
| 2 | SEQ ID NO: 8 | SEQ ID NO: 7 | SEQ ID NO: 2 | SEQ ID NO: 1 |
| 3 | SEQ ID NO: 8 | SEQ ID NO: 7 | SEQ ID NO: 4 | SEQ ID NO: 3 |
| 4 | SEQ ID NO: 8 | SEQ ID NO: 7 | SEQ ID NO: 6 | SEQ ID NO: 5 |
| 5 | SEQ ID NO: 8 | SEQ ID NO: 7 | SEQ ID NO: 4 | SEQ ID NO: 5 |
| 6 | SEQ ID NO: 8 | SEQ ID NO: 7 | SEQ ID NO: 6 | SEQ ID NO: 3 |
| 7 | SEQ ID NO: 4 | SEQ ID NO: 3 | SEQ ID NO: 8 | SEQ ID NO: 7 |
| 8 | SEQ ID NO: 4 | SEQ ID NO: 5 | SEQ ID NO: 8 | SEQ ID NO: 7 |
| 9 | SEQ ID NO: 6 | SEQ ID NO: 3 | SEQ ID NO: 8 | SEQ ID NO: 7 |
| 10 | SEQ ID NO: 6 | SEQ ID NO: 5 | SEQ ID NO: 8 | SEQ ID NO: 7 |

In some embodiments, the compound comprises a VL1 sequence comprising a first light chain CDR1, CDR2, and CDR3 and a VH1 sequence comprising a first heavy chain CDR1, CDR2, and CDR3, a VL2 sequence comprising a second light chain CDR1, CDR2 and CDR3, and a VH2 sequence comprising a second heavy chain CDR1, CDR2, and CDR3. In some embodiments, the CDRs are the CDRs of one or more VL1, VH1, VL2, and VH2 sequences provided in Table 1 or Table 2A. Exemplary light chain and heavy chain CDR sequences for the VL1, VH1, VL2, and VH2 sequences provided in Table 1 are shown below:

SEQ ID NO: 1 CDRs:
(CDR1)
(SEQ ID NO: 146)
DYAMH,
(CDR2)
(SEQ ID NO: 147)
AITWNSGHIDYADSVEG,
(CDR3)
(SEQ ID NO: 148)
VSYLSTASSLDY
SEQ ID NO: 2 CDRs:
(CDR1)
(SEQ ID NO: 149)
RASQGIRNYLA,
(CDR2)
(SEQ ID NO: 150)
AASTLQS,
(CDR3)
(SEQ ID NO: 151)
QRYNRAPYT
SEQ ID NO: 3 and SEQ ID NO: 5 CDRs:
(CDR1)
(SEQ ID NO: 152)
SYAMH,
(CDR2)
(SEQ ID NO: 153)
FMSYDGSNKKYADSVKG,
(CDR3)
(SEQ ID NO: 154)
NYYYYGMDV
SEQ ID NO: 4 and SEQ ID NO: 6 CDRs:
(CDR1)
(SEQ ID NO: 155)
RASQSVYSYLA,
(CDR2)
(SEQ ID NO: 156)
DASNRAT,
(CDR3)
(SEQ ID NO: 157)
QQRSNWPPFT
SEQ ID NO: 7 CDRs:
(CDR1)
(SEQ ID NO: 158)
DQTIH,
(CDR2)
(SEQ ID NO: 159)
YIYPRDDSPKYNENFKG,
(CDR3)
(SEQ ID NO: 160)
PDRSGYAWFIY
SEQ ID NO: 8 CDRs:
(CDR1)
(SEQ ID NO: 161)
KASRDVAIAVA,
(CDR2)
(SEQ ID NO: 162)
WASTRHT,
(CDR3)
(SEQ ID NO: 163)
HQYSSYPFT

In some embodiments, the compound comprises a VH1, VL1, VH2, and/or VL2 that comprises a sequence that is at least 80% (e.g., 85%, 90%, 95%, 96%, 97%, 98%, or 99%) identical to a sequence described in Table 1. The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, the compound comprises a VH1, VL1, VH2, and/or VL2 that comprises a sequence comprising one or more (e.g., 1, 2, 3, 4, 5, 6, 7, 8, 9, 10 or more) mutations in a sequence described in Table 1. Such mutations can be conservative amino acid substitutions. As used herein, a “conservative amino acid substitution” refers to an amino acid substitution that does not alter the relative charge or size characteristics of the protein in which the amino acid substitution is made. Conservative substitutions of amino acids include, for example, substitutions made amongst amino acids within the following groups: (a) M, I, L, V; (b) F, Y, W; (c) K, R, H; (d) A, G; (e) S, T; (f) Q, N; and (g) E, D.

The amino acid sequences of the hinge region, CH2 and CH3 of the compound (and optionally the CH1 and CL, if the compound contains such regions) may be derived from any appropriate source, e.g., a constant region of an antibody such as an IgG1, IgG2, IgG3, or IgG4. Antibody heavy and light chain constant regions amino acid sequences are well known in the art, e.g., those provided in the IMGT database (www.imgt.org) or at www.vbase2.org/vbstat.php., both of which are incorporated by reference herein. In some embodiments, the amino acid sequences of the CH2 and CH3 are derived from an IgG1 or an IgG4 (e.g., SEQ ID NO: 39 or 37). In some embodiments, the CL comprises the amino acid sequence of a kappa CL or a lambda CL. In some embodiments, the hinge region comprises the amino acid sequence EPKSCDKTHTCPPCP (SEQ ID NO:40).

In some embodiments, the CH2 and/or CH3 of the compound (and optionally the CH1 and CL, if the compound contains such regions) may comprise one or more amino acid substitutions that differ from a wild type CH2 or CH3, e.g., one or more amino acid substitutions in a wild type IgG1 CH2 or CH3 or one or more amino acid substitutions in a wild type IgG4 CH2 or CH3 (SEQ ID NO: 39 provides an exemplary wild-type IgG1). Such substitutions are known in the art (see, e.g., U.S. Pat. Nos. 7,704,497, 7,083,784, 6,821,505, 8,323,962, 6,737,056, and 7,416,727).

In some embodiments, the CH2 comprises an amino acid substitution at 234, 235, 252, 254, and/or 256, numbered according to the EU index as in Kabat for a conventional antibody (Kabat et al. Sequences of Proteins of Immunological Interest, U.S. Department of Health and Human Services, 1991, which is incorporated by reference herein in its entirety). It is to be understood that all amino acid positions described herein refer to the numbering of the EU index as in Kabat for a conventional antibody. In some embodiments, the CH2 comprises an amino acid substitution at position 252, 254, and/or 256. In some embodiments, the amino acid at position 252 is tyrosine, phenylalanine, serine, tryptophan, or threonine. In some embodiments, the amino acid at position 254 is threonine. In some embodiments, the amino acid at position 254 is serine, arginine, glutamine, glutamic acid, or aspartic acid. In some embodiments, the CH2 comprises a tyrosine at position 252, a threonine at position 254 and a glutamic acid a position 256 (referred to herein as a YTE mutant). In some embodiments, the CH2 comprises an amino acid substitution at position 234 and/or 235. In some embodiments, the CH2 comprises an alanine at position 234 and an alanine at position 235, also referred to herein as KO mutant. In some embodiments, the CH2 comprises a tyrosine at position 252, a threonine at position 254, a glutamic acid a position 256, an alanine at position 234 and an alanine at position 235, also referred to herein as KO-YTE mutant.

In some embodiments, one or more linkers may be used to connect domains/regions together on the first and/or second polypeptide. For example, the first polypeptide may comprise a linker between the VL1 and VH2. If the first polypeptide comprises a CH1, the first polypeptide may comprise a linker between the VL1 or VH2 (depending on the configuration discussed above) and the CH1 (e.g., VL1-linker-CH1 or VH2-linker-CH1). In another example, the second polypeptide may comprise a linker between the VL2 and VH1. If the second polypeptide further comprises a CL, the second polypeptide may further comprise a linker between the VL2 or VH1 (depending on the configuration discussed above) and the CL (e.g., VL2-linker-CL or VH1-linker-CL). It is to be understood that any number of linkers may be used to connect any domain or region to any other another domain or region on the first polypeptide and/or that any number of linkers may be used to connect any domain or region to any other another domain or region on the second polypeptide.

Any suitable linker known in the art is contemplated for use herein. In some embodiments, the linker is a peptide linker. In some embodiments, the peptide linker comprises at least two amino acids, e.g., 2, 3, 4, 5, 6, 7, 8, 9, 10 or more amino acids. In some embodiments, the peptide linker is no more than 50, 40, 30, 25, 20, 19, 18, 17, 16, 15, 14, 13, 12, 11, 10, 9, 8, 7, 6, 5, 4, 3, or 2 amino acids in length. In some embodiments, the peptide linker is between 2 and 50, 2 and 40, 2 and 30, 2 and 20, 2 and 10, 2 and 9, 2 and 8, 2 and 7, or 2 and 6 amino acids in length. In some embodiments, the peptide linker comprises the amino acid sequence GGGSGGG (SEQ ID NO:9), LGGGSG (SEQ ID NO:10), FNRGES (SEQ ID NO:11), VEPKSS (SEQ ID NO:12), or a combination thereof. In some embodiments, the peptide linker may comprise multiple copies of a linker sequence, e.g., multiple copies of the sequence GGGSGGG (SEQ ID NO:9), LGGGSG (SEQ ID NO:10), FNRGES (SEQ ID NO:11), VEPKSS (SEQ ID NO:12), or a combination thereof.

In some embodiments, the compound comprises two first polypeptides and two second polypeptides. In some embodiments, the CH2 and CH3 of one of the first polypeptides associates with the CH2 and CH3 of the other of the first polypeptides to form a tetravalent molecule (e.g., the two first polypeptides dimerize through associations between their respective CH2 and CH3 domains to form a tetravalent molecule comprising two binding sites specific for the first target protein and two binding sites specific for the second target protein). If the first polypeptide further comprises a CH1 domain, the CH1 domain may also participate in formation of a tetravalent molecule (e.g., the two first polypeptides dimerize through associations between their respective CH1, CH2 and CH3 domains to form a tetravalent molecule comprising two binding sites for the first target protein and two binding sites for the second target protein). In some embodiments, the two first polypeptides are associated together via at least one disulfide bond.

Also contemplated herein are other compounds that compete for binding with a compound as described herein, e.g., a test compound that competes with a compound as described herein for binding to TNF-alpha and IL23A. In some embodiments, the test compound may have at least 80%, 85%, 90%, 95%, 96%, 97%, 98%, or 99% sequence identity with a compound as described herein. Competitive binding may be determined using any assay known in the art, e.g., equilibrium binding, ELISA, surface plasmon resonance, or spectroscopy.

In some embodiments, the compound described herein specifically binds to both TNF-alpha and IL23A. A compound that “specifically binds” to an antigen or an epitope is a term well understood in the art, and methods to determine such specific binding are also well known in the art. A molecule is said to exhibit “specific binding” if it reacts or associates more frequently, more rapidly, with greater duration and/or with greater affinity with a particular target antigen than it does with alternative targets. A compound “specifically binds” to a target antigen or epitope if it binds with greater affinity, avidity, more readily, and/or with greater duration than it binds to other substances. For example, a compound that specifically (or preferentially) binds to an antigen (e.g., TNF-alpha or IL23A) or an antigenic epitope therein is a compound that binds this target antigen with greater affinity, avidity, more readily, and/or with greater duration than it binds to other antigens or other epitopes in the same antigen. It is also understood by reading this definition that, for example, a compound that specifically binds to a first target antigen may or may not specifically or preferentially bind to a second target antigen. As such, “specific binding” or “preferential binding” does not necessarily require (although it can include) exclusive binding. Generally, but not necessarily, reference to binding means preferential binding. In some examples, a compound that “specifically binds” to a target antigen or an epitope thereof may not bind to other antigens or other epitopes in the same antigen.

In some embodiments, a compound as described herein has a suitable binding affinity for TNF-alpha and IL23 or antigenic epitopes thereof. As used herein, “binding affinity” refers to the apparent association constant or K_{A}. The K_{A} is the reciprocal of the dissociation constant (K_{D}). The compound described herein may have a binding affinity (K_{D}) of at least 10⁻⁵, 10⁻⁶, 10⁻⁷, 10⁻⁸, 10⁻⁹, 10⁻¹⁰, 10⁻¹¹, 10⁻¹² M or lower for one or both of the target antigens or antigenic epitopes. An increased binding affinity corresponds to a decreased K_{D}. In some embodiments, the compound described herein has a binding affinity (K_{D}) of at least 10⁻¹¹ M or lower for one or both of the target antigens or antigenic epitopes. Higher affinity binding of a compound for a first antigen and a second antigen relative to a third antigen can be indicated by a higher K_{A} (or a smaller numerical value K_{D}) for binding the first antigen and second antigen than the K_{A} (or numerical value K_{D}) for binding the third antigen. In such cases, the compound has specificity for the first antigen and second antigen (e.g., a first protein in a first conformation or mimic thereof and a second protein in a first conformation or mimic thereof) relative to the third antigen (e.g., the same first or second protein in a second conformation or mimic thereof; or a third protein). Differences in binding affinity (e.g., for specificity or other comparisons) can be at least 1.5, 2, 3, 4, 5, 10, 15, 20, 37.5, 50, 70, 80, 91, 100, 500, 1000, 10,000 or 10⁵ fold.

Binding affinity (or binding specificity) can be determined by a variety of methods including, equilibrium binding, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is related to the concentration of free target protein ([Free]) and the concentration of binding sites for the binding protein on the target where (N) is the number of binding sites per target molecule by the following equation:

$$[ Bound ] = [ N ] [ Fre e ] / ( Kd + [ Free ] )$$

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

In some embodiments, the compound comprises a first polypeptide and a second polypeptide as defined in Table 2A. In some embodiments, the compound comprises:
(i) a first polypeptide comprises the amino acid sequence of SEQ ID NO:13 and a second polypeptide comprises the amino acid sequence of SEQ ID NO:14;
(ii) a first polypeptide comprises the amino acid sequence of SEQ ID NO:15 and a second polypeptide comprises the amino acid sequence of SEQ ID NO:16;
(iii) a first polypeptide comprises the amino acid sequence of SEQ ID NO:17 and a second polypeptide comprises the amino acid sequence of SEQ ID NO:18;
(iv) a first polypeptide comprises the amino acid sequence of SEQ ID NO:19 and a second polypeptide comprises the amino acid sequence of SEQ ID NO:20;
(v) a first polypeptide comprises the amino acid sequence of SEQ ID NO:21 and a second polypeptide comprises the amino acid sequence of SEQ ID NO:22;
(vi) a first polypeptide comprises the amino acid sequence of SEQ ID NO:23 and a second polypeptide comprises the amino acid sequence of SEQ ID NO:24;
(vii) a first polypeptide comprises the amino acid sequence of SEQ ID NO:25 and a second polypeptide comprises the amino acid sequence of SEQ ID NO:26;
(viii) a first polypeptide comprises the amino acid sequence of SEQ ID NO:27 and a second polypeptide comprises the amino acid sequence of SEQ ID NO:28;
(ix) a first polypeptide comprises the amino acid sequence of SEQ ID NO:29 and a second polypeptide comprises the amino acid sequence of SEQ ID NO:30;
(x) a first polypeptide comprises the amino acid sequence of SEQ ID NO:31 and a second polypeptide comprises the amino acid sequence of SEQ ID NO:32;
(xi) a first polypeptide comprises the amino acid sequence of SEQ ID NO:33 and a second polypeptide comprises the amino acid sequence of SEQ ID NO:34;
(xii) a first polypeptide comprises the amino acid sequence of SEQ ID NO:35 and a second polypeptide comprises the amino acid sequence of SEQ ID NO:36;
(xiii) a first polypeptide comprises the amino acid sequence of SEQ ID NO:44 and a second polypeptide comprises the amino acid sequence of SEQ ID NO:45;
(xiv) a first polypeptide comprises the amino acid sequence of SEQ ID NO:46 and a second polypeptide comprises the amino acid sequence of SEQ ID NO:47;
(xv) a first polypeptide comprises the amino acid sequence of SEQ ID NO:48 and a second polypeptide comprises the amino acid sequence of SEQ ID NO:49;
(xvi) a first polypeptide comprises the amino acid sequence of SEQ ID NO:50 and a second polypeptide comprises the amino acid sequence of SEQ ID NO:51;
(xvii) a first polypeptide comprises the amino acid sequence of SEQ ID NO:52 and a second polypeptide comprises the amino acid sequence of SEQ ID NO:53;
(xviii) a first polypeptide comprises the amino acid sequence of SEQ ID NO:54 and a second polypeptide comprises the amino acid sequence of SEQ ID NO:55;
(xix) a first polypeptide comprises the amino acid sequence of SEQ ID NO:56 and a second polypeptide comprises the amino acid sequence of SEQ ID NO:57;
(xx) a first polypeptide comprises the amino acid sequence of SEQ ID NO:58 and a second polypeptide comprises the amino acid sequence of SEQ ID NO:59;
(xxi) a first polypeptide comprises the amino acid sequence of SEQ ID NO:60 and a second polypeptide comprises the amino acid sequence of SEQ ID NO:61;
(xxii) a first polypeptide comprises the amino acid sequence of SEQ ID NO:62 and a second polypeptide comprises the amino acid sequence of SEQ ID NO:63;
(xxiii) a first polypeptide comprises the amino acid sequence of SEQ ID NO:64 and a second polypeptide comprises the amino acid sequence of SEQ ID NO:65;
(xxiv) a first polypeptide comprises the amino acid sequence of SEQ ID NO:66 and a second polypeptide comprises the amino acid sequence of SEQ ID NO:67;
(xxv) a first polypeptide comprises the amino acid sequence of SEQ ID NO:68 and a second polypeptide comprises the amino acid sequence of SEQ ID NO:69;
(xxvi) a first polypeptide comprises the amino acid sequence of SEQ ID NO:70 and a second polypeptide comprises the amino acid sequence of SEQ ID NO:71;
(xxvii) a first polypeptide comprises the amino acid sequence of SEQ ID NO:72 and a second polypeptide comprises the amino acid sequence of SEQ ID NO:73;
(xxviii) a first polypeptide comprises the amino acid sequence of SEQ ID NO:74 and a second polypeptide comprises the amino acid sequence of SEQ ID NO:75;
(xxix) a first polypeptide comprises the amino acid sequence of SEQ ID NO:76 and a second polypeptide comprises the amino acid sequence of SEQ ID NO:77;
(xxx) a first polypeptide comprises the amino acid sequence of SEQ ID NO:78 and a second polypeptide comprises the amino acid sequence of SEQ ID NO:79;
(xxxi) a first polypeptide comprises the amino acid sequence of SEQ ID NO:80 and a second polypeptide comprises the amino acid sequence of SEQ ID NO:81;
(xxxii) a first polypeptide comprises the amino acid sequence of SEQ ID NO:82 and a second polypeptide comprises the amino acid sequence of SEQ ID NO:83;
(xxxiii) a first polypeptide comprises the amino acid sequence of SEQ ID NO:84 and a second polypeptide comprises the amino acid sequence of SEQ ID NO:85;
(xxxiv) a first polypeptide comprises the amino acid sequence of SEQ ID NO:86 and a second polypeptide comprises the amino acid sequence of SEQ ID NO:87;
(xxxv) a first polypeptide comprises the amino acid sequence of SEQ ID NO:88 and a second polypeptide comprises the amino acid sequence of SEQ ID NO:89;
(xxxvi) a first polypeptide comprises the amino acid sequence of SEQ ID NO:90 and a second polypeptide comprises the amino acid sequence of SEQ ID NO:91;
(xxxvii) a first polypeptide comprises the amino acid sequence of SEQ ID NO:92 and a second polypeptide comprises the amino acid sequence of SEQ ID NO:93;
(xxxviii) a first polypeptide comprises the amino acid sequence of SEQ ID NO:94 and a second polypeptide comprises the amino acid sequence of SEQ ID NO:95;
(xxxix) a first polypeptide comprises the amino acid sequence of SEQ ID NO:96 and a second polypeptide comprises the amino acid sequence of SEQ ID NO:97;
(xl) a first polypeptide comprises the amino acid sequence of SEQ ID NO:98 and a second polypeptide comprises the amino acid sequence of SEQ ID NO:99;
(xli) a first polypeptide comprises the amino acid sequence of SEQ ID NO:100 and a second polypeptide comprises the amino acid sequence of SEQ ID NO:101;
(xlii) a first polypeptide comprises the amino acid sequence of SEQ ID NO:102 and a second polypeptide comprises the amino acid sequence of SEQ ID NO:103;
(xliii) a first polypeptide comprises the amino acid sequence of SEQ ID NO:104 and a second polypeptide comprises the amino acid sequence of SEQ ID NO:105;
(xliv) a first polypeptide comprises the amino acid sequence of SEQ ID NO:106 and a second polypeptide comprises the amino acid sequence of SEQ ID NO:107;
(xlv) a first polypeptide comprises the amino acid sequence of SEQ ID NO:108 and a second polypeptide comprises the amino acid sequence of SEQ ID NO:109;
(xlvi) a first polypeptide comprises the amino acid sequence of SEQ ID NO:110 and a second polypeptide comprises the amino acid sequence of SEQ ID NO:111;
(xlvii) a first polypeptide comprises the amino acid sequence of SEQ ID NO:112 and a second polypeptide comprises the amino acid sequence of SEQ ID NO:113;
(xlviii) a first polypeptide comprises the amino acid sequence of SEQ ID NO:114 and a second polypeptide comprises the amino acid sequence of SEQ ID NO:115;
(xlix) a first polypeptide comprises the amino acid sequence of SEQ ID NO:116 and a second polypeptide comprises the amino acid sequence of SEQ ID NO:117;
(l) a first polypeptide comprises the amino acid sequence of SEQ ID NO:118 and a second polypeptide comprises the amino acid sequence of SEQ ID NO:119;
(li) a first polypeptide comprises the amino acid sequence of SEQ ID NO:120 and a second polypeptide comprises the amino acid sequence of SEQ ID NO:121;
(lii) a first polypeptide comprises the amino acid sequence of SEQ ID NO:122 and a second polypeptide comprises the amino acid sequence of SEQ ID NO:123;
(liii) a first polypeptide comprises the amino acid sequence of SEQ ID NO:124 and a second polypeptide comprises the amino acid sequence of SEQ ID NO:125;
(liv) a first polypeptide comprises the amino acid sequence of SEQ ID NO:126 and a second polypeptide comprises the amino acid sequence of SEQ ID NO:127;
(lv) a first polypeptide comprises the amino acid sequence of SEQ ID NO:128 and a second polypeptide comprises the amino acid sequence of SEQ ID NO:129;
(lvi) a first polypeptide comprises the amino acid sequence of SEQ ID NO:130 and a second polypeptide comprises the amino acid sequence of SEQ ID NO:131;
(lvii) a first polypeptide comprises the amino acid sequence of SEQ ID NO:132 and a second polypeptide comprises the amino acid sequence of SEQ ID NO:133;
(lviii) a first polypeptide comprises the amino acid sequence of SEQ ID NO:134 and a second polypeptide comprises the amino acid sequence of SEQ ID NO:135;
(lix) a first polypeptide comprises the amino acid sequence of SEQ ID NO:136 and a second polypeptide comprises the amino acid sequence of SEQ ID NO:137;
(lx) a first polypeptide comprises the amino acid sequence of SEQ ID NO:138 and a second polypeptide comprises the amino acid sequence of SEQ ID NO:139;
(lxi) a first polypeptide comprises the amino acid sequence of SEQ ID NO:140 and a second polypeptide comprises the amino acid sequence of SEQ ID NO:141; or
(lxii) a first polypeptide comprises the amino acid sequence of SEQ ID NO:142 and a second polypeptide comprises the amino acid sequence of SEQ ID NO:143.

In some embodiments, the compound comprises:
(i) a first polypeptide comprises the amino acid sequence of SEQ ID NO:13 and a second polypeptide comprises the amino acid sequence of SEQ ID NO:14;
(ii) a first polypeptide comprises the amino acid sequence of SEQ ID NO:15 and a second polypeptide comprises the amino acid sequence of SEQ ID NO:16;
(iii) a first polypeptide comprises the amino acid sequence of SEQ ID NO:17 and a second polypeptide comprises the amino acid sequence of SEQ ID NO:18;
(iv) a first polypeptide comprises the amino acid sequence of SEQ ID NO:19 and a second polypeptide comprises the amino acid sequence of SEQ ID NO:20;
(v) a first polypeptide comprises the amino acid sequence of SEQ ID NO:21 and a second polypeptide comprises the amino acid sequence of SEQ ID NO:22;
(vi) a first polypeptide comprises the amino acid sequence of SEQ ID NO:23 and a second polypeptide comprises the amino acid sequence of SEQ ID NO:24;
(vii) a first polypeptide comprises the amino acid sequence of SEQ ID NO:25 and a second polypeptide comprises the amino acid sequence of SEQ ID NO:26;
(viii) a first polypeptide comprises the amino acid sequence of SEQ ID NO:27 and a second polypeptide comprises the amino acid sequence of SEQ ID NO:28;
(ix) a first polypeptide comprises the amino acid sequence of SEQ ID NO:29 and a second polypeptide comprises the amino acid sequence of SEQ ID NO:30;
(x) a first polypeptide comprises the amino acid sequence of SEQ ID NO:31 and a second polypeptide comprises the amino acid sequence of SEQ ID NO:32;
(xi) a first polypeptide comprises the amino acid sequence of SEQ ID NO:33 and a second polypeptide comprises the amino acid sequence of SEQ ID NO:34; or
(xii) a first polypeptide comprises the amino acid sequence of SEQ ID NO:35 and a second polypeptide comprises the amino acid sequence of SEQ ID NO:36.

### Methods of Producing Compounds, Nucleic Acids, Vectors, and Cells

Aspects of the disclosure also include nucleic acids that encode compounds described herein or polypeptides described herein (e.g., first or second polypeptides described herein), which may be encoded together or separately. The polynucleotides encoding the compounds described herein or polypeptides described herein may be obtained, and the nucleotide sequence of the polynucleotides determined, by any method known in the art.

In some embodiments, the nucleic acid is comprised within a vector, such as an expression vector. In some embodiments, the vector comprises a promoter operably linked to the nucleic acid.

A variety of promoters can be used for expression of the compounds described herein or polypeptides described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using estradiol, RU486, diphenol murislerone, or rapamycin. Inducible systems are available from Invitrogen, Clontech and Ariad.

Regulatable promoters that include a repressor with the operon can be used. In one embodiment, the lac repressor from Escherichia coli can function as a transcriptional modulator to regulate transcription from lac operator-bearing mammalian cell promoters [M. Brown et al., Cell, 49:603-612 (1987)]; Gossen and Bujard (1992); [M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used (Yao et al., Human Gene Therapy; Gossen et al., Natl. Acad. Sci. USA, 89:5547-5551 (1992); Shockett et al., Proc. Natl. Acad. Sci. USA, 92:6522-6526 (1995)).

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

An expression vector comprising the nucleic acid can be transferred to a host cell by conventional techniques (e.g., electroporation, liposomal transfection, and calcium phosphate precipitation) and the transfected cells are then cultured by conventional techniques to produce the compounds described herein. In some embodiments, the expression of the compounds described herein is regulated by a constitutive, an inducible or a tissue-specific promoter.

The host cells used to express the compounds described herein or polypeptides described herein may be either bacterial cells such as Escherichia coli, or, preferably, eukaryotic cells. In particular, mammalian cells, such as Chinese hamster ovary cells (CHO), in conjunction with a vector such as the major intermediate early gene promoter element from human cytomegalovirus is an effective expression system for immunoglobulins (Foecking et al. (1986) “Powerful And Versatile Enhancer-Promoter Unit For Mammalian Expression Vectors,” Gene 45:101-106; Cockett et al. (1990) “High Level Expression Of Tissue Inhibitor Of Metalloproteinases In Chinese Hamster Ovary Cells Using Glutamine Synthetase Gene Amplification,” Biotechnology 8:662-667).

A variety of host-expression vector systems may be utilized to express the compounds described herein or polypeptides described herein. Such host-expression systems represent vehicles by which the coding sequences of the compounds described herein or polypeptides described herein may be produced and subsequently purified, but also represent cells which may, when transformed or transfected with the appropriate nucleotide coding sequences, express the compounds described herein in situ. These include, but are not limited to, microorganisms such as bacteria (e.g., E. coli and B. subtilis) transformed with recombinant bacteriophage DNA, plasmid DNA or cosmid DNA expression vectors containing coding sequences for the compounds described herein; yeast (e.g., Saccharomyces pichia) transformed with recombinant yeast expression vectors containing sequences encoding the compounds described herein; insect cell systems infected with recombinant virus expression vectors (e.g., baclovirus) containing the sequences encoding the compounds described herein; plant cell systems infected with recombinant virus expression vectors (e.g., cauliflower mosaic virus (CaMV) and tobacco mosaic virus (TMV) or transformed with recombinant plasmid expression vectors (e.g., Ti plasmid) containing sequences encoding the molecules compounds described herein; or mammalian cell systems (e.g., COS, CHO, BHK, 293, 293T, 3T3 cells, lymphotic cells (see U.S. Pat. No. 5,807,715), Per C.6 cells (human retinal cells developed by Crucell) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the compound being expressed. For example, when a large quantity of such a protein is to be produced, for the generation of pharmaceutical compositions of compounds described herein, vectors which direct the expression of high levels of fusion protein products that are readily purified may be desirable. Such vectors include, but are not limited, to the E. coli expression vector pUR278 (Ruther et al. (1983) “Easy Identification Of cDNA Clones,” EMBO J. 2:1791-1794), in which the coding sequence may be ligated individually into the vector in frame with the lac Z coding region so that a fusion protein is produced; pIN vectors (Inouye et al. (1985) “Up-Promoter Mutations In The lpp Gene Of Escherichia coli,” Nucleic Acids Res. 13:3101-3110; Van Heeke et al. (1989) “Expression Of Human Asparagine Synthetase In Escherichia coli,” J. Biol. Chem. 24:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The coding sequence may be cloned individually into non-essential regions (e.g., the polyhedrin gene) of the virus and placed under control of an AcNPV promoter (e.g., the polyhedrin promoter).

In mammalian host cells, a number of viral-based expression systems may be utilized. In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the immunoglobulin molecule in infected hosts (e.g., see Logan et al. (1984) “Adenovirus Tripartite Leader Sequence Enhances Translation Of mRNAs Late After Infection,” Proc. Natl. Acad. Sci. USA 81:3655-3659). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bitter et al. (1987) “Expression And Secretion Vectors For Yeast,” Methods in Enzymol. 153:516-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. For example, in certain embodiments, the compounds described herein may be expressed as a single gene product (e.g., as a single polypeptide chain, i.e., as a polyprotein precursor), requiring proteolytic cleavage by native or recombinant cellular mechanisms to form separate polypeptides of the compounds described herein. The disclosure thus encompasses engineering a nucleic acid sequence to encode a polyprotein precursor molecule comprising the polypeptides of the compounds described herein, which includes coding sequences capable of directing post translational cleavage of said polyprotein precursor. Post-translational cleavage of the polyprotein precursor results in the polypeptides of the compounds described herein. The post translational cleavage of the precursor molecule comprising the polypeptides of the compounds described herein may occur in vivo (i.e., within the host cell by native or recombinant cell systems/mechanisms, e.g. furin cleavage at an appropriate site) or may occur in vitro (e.g. incubation of said polypeptide chain in a composition comprising proteases or peptidases of known activity and/or in a composition comprising conditions or reagents known to foster the desired proteolytic action). Purification and modification of recombinant proteins is well known in the art such that the design of the polyprotein precursor could include a number of embodiments readily appreciated by a skilled worker. Any known proteases or peptidases known in the art can be used for the described modification of the precursor molecule, e.g., thrombin or factor Xa (Nagai et al. (1985) “Oxygen Binding Properties Of Human Mutant Hemoglobins Synthesized In Escherichia coli,” Proc. Nat. Acad. Sci. USA 82:7252-7255, and reviewed in Jenny et al. (2003) “A Critical Review Of The Methods For Cleavage Of Fusion Proteins With Thrombin And Factor Xa,” Protein Expr. Purif. 31:1-11, each of which is incorporated by reference herein in its entirety)), enterokinase (Collins-Racie et al. (1995) “Production Of Recombinant Bovine Enterokinase Catalytic Subunit In Escherichia coli Using The Novel Secretory Fusion Partner DsbA,” Biotechnology 13:982-987 hereby incorporated by reference herein in its entirety)), furin, and AcTEV (Parks et al. (1994) “Release Of Proteins And Peptides From Fusion Proteins Using A Recombinant Plant Virus Proteinase,” Anal. Biochem. 216:413-417 hereby incorporated by reference herein in its entirety)) and the Foot and Mouth Disease Virus Protease C3.

Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, HeLa, COS, MDCK, 293, 293T, 3T3, WI38, BT483, Hs578T, HTB2, BT20 and T47D, CRL7030 and Hs578Bst.

For long-term, high-yield production of recombinant proteins, stable expression is preferred. For example, cell lines which stably express compounds described herein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the compounds described herein. Such engineered cell lines may be particularly useful in screening and evaluation of compounds that interact directly or indirectly with the compounds described herein.

A number of selection systems may be used, including but not limited to the herpes simplex virus thymidine kinase (Wigler et al. (1977) “Transfer Of Purified Herpes Virus Thymidine Kinase Gene To Cultured Mouse Cells,” Cell 11: 223-232), hypoxanthine-guanine phosphoribosyltransferase (Szybalska et al. (1992) “Use Of The HPRT Gene And The HAT Selection Technique In DNA-Mediated Transformation Of Mammalian Cells First Steps Toward Developing Hybridoma Techniques And Gene Therapy,” Bioessays 14: 495-500), and adenine phosphoribosyltransferase (Lowy et al. (1980) “Isolation Of Transforming DNA: Cloning The Hamster aprt Gene,” Cell 22: 817-823) genes can be employed in tk−, hgprt− or aprt− cells, respectively. Also, antimetabolite resistance can be used as the basis of selection for the following genes: dhfr, which confers resistance to methotrexate (Wigler et al. (1980) “Transformation Of Mammalian Cells With An Amplifiable Dominant-Acting Gene,” Proc. Natl. Acad. Sci. USA 77:3567-3570; O'Hare et al. (1981) “Transformation Of Mouse Fibroblasts To Methotrexate Resistance By A Recombinant Plasmid Expressing A Prokaryotic Dihydrofolate Reductase,” Proc. Natl. Acad. Sci. USA 78: 1527-1531); gpt, which confers resistance to mycophenolic acid (Mulligan et al. (1981) “Selection For Animal Cells That Express The Escherichia coli Gene Coding For Xanthine-Guanine Phosphoribosyltransferase,” Proc. Natl. Acad. Sci. USA 78: 2072-2076); neo, which confers resistance to the aminoglycoside G-418 (Tolstoshev (1993) “Gene Therapy, Concepts, Current Trials And Future Directions,” Ann. Rev. Pharmacol. Toxicol. 32:573-596; Mulligan (1993) “The Basic Science Of Gene Therapy,” Science 260:926-932; and Morgan et al. (1993) “Human Gene Therapy,” Ann. Rev. Biochem. 62:191-217) and hygro, which confers resistance to hygromycin (Santerre et al. (1984) “Expression Of Prokaryotic Genes For Hygromycin B And G418 Resistance As Dominant-Selection Markers In Mouse L Cells,” Gene 30:147-156). Methods commonly known in the art of recombinant DNA technology which can be used are described in Ausubel et al. (eds.), 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al. (1981) “A New Dominant Hybrid Selective Marker For Higher Eukaryotic Cells,” J. Mol. Biol. 150:1-14.

The expression levels of compounds described herein or polypeptides described herein can be increased by vector amplification (for a review, see Bebbington and Hentschel, The use of vectors based on gene amplification for the expression of cloned genes in mammalian cells in DNA cloning, Vol. 3 (Academic Press, New York, 1987). When a marker in the vector system expressing a compound described herein is amplifiable, increase in the level of inhibitor present in culture of host cell will increase the number of copies of the marker gene. Since the amplified region is associated with the nucleotide sequence of a compound described herein or a polypeptide described herein, production of the polypeptide will also increase (Crouse et al. (1983) “Expression And Amplification Of Engineered Mouse Dihydrofolate Reductase Minigenes,” Mol. Cell. Biol. 3:257-266).

The host cell may be co-transfected with two expression vectors, the first vector encoding the first polypeptide of a compound described herein and the second vector encoding the second polypeptide of a compound described herein. The two vectors may contain identical selectable markers which enable equal expression of both polypeptides. Alternatively, a single vector may be used which encodes both polypeptides. The coding sequences for the polypeptides of compounds described herein may comprise cDNA or genomic DNA.

Once a compound described herein or polypeptide described herein has been recombinantly expressed, it may be purified by any method known in the art for purification of polypeptides, polyproteins or antibodies (e.g., analogous to antibody purification schemes based on antigen selectivity) for example, by chromatography (e.g., ion exchange, affinity, particularly by affinity for the specific antigen (optionally after Protein A selection where the compound comprises an Fc domain (or portion thereof)), and sizing column chromatography), centrifugation, differential solubility, or by any other standard technique for the purification of polypeptides or antibodies.

Other aspects of the disclosure relate to a cell comprising a nucleic acid described herein or a vector described herein. The cell may be a prokaryotic or eukaryotic cell. In some embodiments, the cell in a mammalian cell. Exemplary cell types are described herein.

Yet other aspects of the disclosure relate to a method of producing a compound described herein or a polypeptide described herein (e.g., a first polypeptide or a second polypeptide), the method comprising obtaining a cell described herein and expressing nucleic acid described herein in said cell. In some embodiments, the method further comprises isolating and purifying a compound described herein or a polypeptide described herein.

### Methods of Treatment and Compositions for Use in Medicine

Other aspects of the disclosure relate to methods of treatment and compositions for use in medicine. Non-limiting examples of compounds for use in such methods and composition are those that comprise:
(i) a first polypeptide comprises the amino acid sequence of SEQ ID NO:13 and a second polypeptide comprises the amino acid sequence of SEQ ID NO:14;
(ii) a first polypeptide comprises the amino acid sequence of SEQ ID NO:15 and a second polypeptide comprises the amino acid sequence of SEQ ID NO:16;
(iii) a first polypeptide comprises the amino acid sequence of SEQ ID NO:17 and a second polypeptide comprises the amino acid sequence of SEQ ID NO:18;
(iv) a first polypeptide comprises the amino acid sequence of SEQ ID NO:19 and a second polypeptide comprises the amino acid sequence of SEQ ID NO:20;
(v) a first polypeptide comprises the amino acid sequence of SEQ ID NO:21 and a second polypeptide comprises the amino acid sequence of SEQ ID NO:22;
(vi) a first polypeptide comprises the amino acid sequence of SEQ ID NO:23 and a second polypeptide comprises the amino acid sequence of SEQ ID NO:24;
(vii) a first polypeptide comprises the amino acid sequence of SEQ ID NO:25 and a second polypeptide comprises the amino acid sequence of SEQ ID NO:26;
(viii) a first polypeptide comprises the amino acid sequence of SEQ ID NO:27 and a second polypeptide comprises the amino acid sequence of SEQ ID NO:28;
(ix) a first polypeptide comprises the amino acid sequence of SEQ ID NO:29 and a second polypeptide comprises the amino acid sequence of SEQ ID NO:30;
(x) a first polypeptide comprises the amino acid sequence of SEQ ID NO:31 and a second polypeptide comprises the amino acid sequence of SEQ ID NO:32;
(xi) a first polypeptide comprises the amino acid sequence of SEQ ID NO:33 and a second polypeptide comprises the amino acid sequence of SEQ ID NO:34; or
(xii) a first polypeptide comprises the amino acid sequence of SEQ ID NO:35 and a second polypeptide comprises the amino acid sequence of SEQ ID NO:36.

In some embodiments, the method of treatment or the use is a method of treating an autoimmune or an inflammatory disease or use in such a method. In some embodiments, the method comprises administering a compound described herein or a pharmaceutical composition comprising said compound to a subject, e.g., a subject having or at risk for having an autoimmune or an inflammatory disease.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human subject having, at risk for, or suspected of having a disease. A subject having a disease can be identified by routine medical examination, e.g., a physical examination, a laboratory test, an organ functional test, a CT scan, or an ultrasound. A subject suspected of having any of such a disease might show one or more symptoms of the disease. Signs and symptoms for diseases, e.g., autoimmune and inflammatory diseases, are well known to those of ordinary skill in the art. A subject at risk for the disease can be a subject having one or more of the risk factors for that disease.

Non-limiting examples of autoimmune diseases include rheumatoid arthritis, psoriasis, type 1 diabetes, systemic lupus erythematosus, transplant rejection, autoimmune thyroid disease (Hashimoto's disease), sarcoidosis, scleroderma, granulomatous vasculitis, Crohn's disease, ulcerative colitis, Sjogren's disease, ankylosing spondylitis, psoriatic arthritis, polymyositis dermatomyositis, polyarteritis nodosa, immunologically mediated blistering skin diseases, Behcet's syndrome, multiple sclerosis, systemic sclerosis, Goodpasture's disease or immune mediated glomerulonephritis.

Non-limiting examples of inflammatory diseases include including rheumatoid arthritis, systemic lupus erythematosus, alopecia areata, anklosing spondylitis, antiphospholipid syndrome, autoimmune Addison's disease, autoimmune hemolytic anemia, autoimmune hepatitis, autoimmune inner ear disease, autoimmune lymphoproliferative syndrome (ALPS), autoimmune thrombocytopenic purpura (ATP), Behcet's disease, bullous pemphigoid, cardiomyopathy, celiac sprue-dermatitis, chronic fatigue syndrome immune deficiency syndrome (CFIDS), chronic inflammatory demyelinating polyneuropathy, cicatricial pemphigoid, cold agglutinin disease, Crest syndrome, Crohn's disease, Dego's disease, dermatomyasitis, dermatomyositis—juvenile, discoid lupus, essential mixed cryoglobulinemia, fibromyalgia—fibromyositis, grave's disease, guillain-barre, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, and Wegener's granulomatosis. In some embodiments, the autoimmune or inflammatory disease is Crohn's disease, ankylosing spondylitis, or psoriatic arthritis.

To practice a method disclosed herein, an effective amount of a compound or pharmaceutical composition described herein can be administered to a subject (e.g., a human) in need of the treatment. Various delivery systems are known and can be used to administer the compounds of the invention. Methods of administration include, but are not limited to, intradermal, intramuscular, intraperitoneal, intravenous, subcutaneous, intranasal, epidural, and oral routes. The compounds of the invention can be administered, for example by infusion, bolus or injection, and can be administered together with other biologically active agents such as anti-inflammatory agents. Administration can be systemic or local. In preferred embodiments, the administration is by subcutaneous injection. Formulations for such injections may be prepared in, for example, prefilled syringes that may be administered once every other week.

“An effective amount” as used herein refers to the amount of each compound required to confer therapeutic effect on the subject, either alone or in combination with one or more other compounds. Effective amounts vary, as recognized by those skilled in the art, depending on the particular condition being treated, the severity of the condition, the individual subject parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a subject may insist upon a lower dose or tolerable dose for medical reasons, psychological reasons or for virtually any other reasons.

Empirical considerations, such as the half-life, generally will contribute to the determination of the dosage. For example, compounds that are compatible with the human immune system, such as compounds comprising regions from humanized antibodies or fully human antibodies, may be used to prolong half-life of the compound and to prevent the compound being attacked by the host's immune system. Frequency of administration may be determined and adjusted over the course of therapy, and is generally, but not necessarily, based on treatment and/or suppression and/or amelioration and/or delay of a disease. Alternatively, sustained continuous release formulations of a compound may be appropriate. Various formulations and devices for achieving sustained release are known in the art.

In some embodiments, dosage is daily, every other day, every three days, every four days, every five days, or every six days. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the compound used) can vary over time.

In some embodiments, for an adult subject of normal weight, doses ranging from about 0.01 to 1000 mg/kg may be administered. In some embodiments, the dose is between 1 to 200 mg. The particular dosage regimen, i.e., dose, timing and repetition, will depend on the particular subject and that subject's medical history, as well as the properties of the compound (such as the half-life of the compound, and other considerations well known in the art).

For the purpose of the present disclosure, the appropriate dosage of a compound as described herein will depend on the specific compound (or compositions thereof) employed, the formulation and route of administration, the type and severity of the disease, whether the compound is administered for preventive or therapeutic purposes, previous therapy, the subject's clinical history and response to the antagonist, and the discretion of the attending physician. Typically the clinician will administer a compound until a dosage is reached that achieves the desired result. Administration of one or more compounds can be continuous or intermittent, depending, for example, upon the recipient's physiological condition, whether the purpose of the administration is therapeutic or prophylactic, and other factors known to skilled practitioners. The administration of a compound may be essentially continuous over a preselected period of time or may be in a series of spaced dose, e.g., either before, during, or after developing a disease.

As used herein, the term “treating” refers to the application or administration of a compound or composition including the compound to a subject, who has a disease, a symptom of the disease, or a predisposition toward the disease, with the purpose to cure, heal, alleviate, relieve, alter, remedy, ameliorate, improve, or affect the disease, the symptom of the disease, or the predisposition toward the disease.

Alleviating a disease includes delaying the development or progression of the disease, or reducing disease severity. Alleviating the disease does not necessarily require curative results. As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a disease includes initial onset and/or recurrence.

In some embodiments, the compound described herein is administered to a subject in need of the treatment at an amount sufficient to inhibit the activity of one or both of TNF-alpha or IL23A by at least 20% (e.g., 30%, 40%, 50%, 60%, 70%, 80%, 90% or greater) in vivo or in vitro. Methods for determining the inhibitory capability of a compound are known in the art. Exemplary TNF-alpha and IL23A inhibition assays are provided in the Examples.

Conventional methods, known to those of ordinary skill in the art of medicine, can be used to administer the compound or pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods.

### Pharmaceutical Compositions

Yet other aspects of the disclosure relate to pharmaceutical compositions comprising a compound described herein. A composition comprising a compound of the invention (e.g., compounds specific for both TNF-alpha and IL23A) can be administered to a subject having or at risk of having an autoimmune or an inflammatory disease. The invention further provides for the use of a compound of the invention in the manufacture of a medicament for treatment of an autoimmune or an inflammatory disease. The compounds can be administered either alone or in combination with other compositions in the prevention or treatment of an autoimmune or an inflammatory disease. Non-limiting examples of compounds of the invention for use in such pharmaceutical compositions are those that comprise:
(i) a first polypeptide comprises the amino acid sequence of SEQ ID NO:13 and a second polypeptide comprises the amino acid sequence of SEQ ID NO:14;
(ii) a first polypeptide comprises the amino acid sequence of SEQ ID NO:15 and a second polypeptide comprises the amino acid sequence of SEQ ID NO:16;
(iii) a first polypeptide comprises the amino acid sequence of SEQ ID NO:17 and a second polypeptide comprises the amino acid sequence of SEQ ID NO:18;
(iv) a first polypeptide comprises the amino acid sequence of SEQ ID NO:19 and a second polypeptide comprises the amino acid sequence of SEQ ID NO:20;
(v) a first polypeptide comprises the amino acid sequence of SEQ ID NO:21 and a second polypeptide comprises the amino acid sequence of SEQ ID NO:22;
(vi) a first polypeptide comprises the amino acid sequence of SEQ ID NO:23 and a second polypeptide comprises the amino acid sequence of SEQ ID NO:24;
(vii) a first polypeptide comprises the amino acid sequence of SEQ ID NO:25 and a second polypeptide comprises the amino acid sequence of SEQ ID NO:26;
(viii) a first polypeptide comprises the amino acid sequence of SEQ ID NO:27 and a second polypeptide comprises the amino acid sequence of SEQ ID NO:28;
(ix) a first polypeptide comprises the amino acid sequence of SEQ ID NO:29 and a second polypeptide comprises the amino acid sequence of SEQ ID NO:30;
(x) a first polypeptide comprises the amino acid sequence of SEQ ID NO:31 and a second polypeptide comprises the amino acid sequence of SEQ ID NO:32;
(xi) a first polypeptide comprises the amino acid sequence of SEQ ID NO:33 and a second polypeptide comprises the amino acid sequence of SEQ ID NO:34; or
(xii) a first polypeptide comprises the amino acid sequence of SEQ ID NO:35 and a second polypeptide comprises the amino acid sequence of SEQ ID NO:36.

As used herein, the term “pharmaceutical composition” refers to the formulation of a compound described herein in combination with a pharmaceutically acceptable carrier. The pharmaceutical composition can further comprise additional agents (e.g. for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, a compound of the invention in a composition is administered by injection, by means of a catheter, by means of a suppository, or by means of an implant, the implant being of a porous, non-porous, or gelatinous material, including a membrane, such as a sialastic membrane, or a fiber. Typically, when administering the composition, materials to which the compound of the invention does not absorb are used.

In other embodiments, the compounds of the invention are delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

Compounds of the invention can be administered as pharmaceutical compositions comprising a therapeutically effective amount of a binding agent and one or more pharmaceutically compatible ingredients.

In typical embodiments, the pharmaceutical composition is formulated in accordance with routine procedures as a pharmaceutical composition adapted for intravenous or subcutaneous administration to a subject, e.g., a human being. Typically, compositions for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in ‘stabilized plasmid-lipid particles’ (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757.

The pharmaceutical compositions of this disclosure may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

In some embodiments, a compound described herein may be conjugated to a therapeutic moiety, e.g., an anti-inflammatory agent. Techniques for conjugating such therapeutic moieties to polypeptides, including e.g., Fc domains, are well known; see, e.g., Amon et al., “Monoclonal Antibodies For Immunotargeting Of Drugs In Cancer Therapy”, in Monoclonal Antibodies And Cancer Therapy, Reisfeld et al. (eds.), 1985, pp. 243-56, Alan R. Liss, Inc.); Hellstrom et al., “Antibodies For Drug Delivery”, in Controlled Drug Delivery (2nd Ed.), Robinson et al. (eds.), 1987, pp. 623-53, Marcel Dekker, Inc.); Thorpe, “Antibody Carriers Of Cytotoxic Agents In Cancer Therapy: A Review”, in Monoclonal Antibodies '84: Biological And Clinical Applications, Pinchera et al. (eds.), 1985, pp. 475-506); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), 1985, pp. 303-16, Academic Press; and Thorpe et al. (1982) “The Preparation And Cytotoxic Properties Of Antibody-Toxin Conjugates,” Immunol. Rev., 62:119-158.

Further, the pharmaceutical composition can be provided as a pharmaceutical kit comprising (a) a container containing a compound of the invention in lyophilized form and (b) a second container containing a pharmaceutically acceptable diluent (e.g., sterile water) for injection. The pharmaceutically acceptable diluent can be used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1. Construction of Exemplary Compounds Targeting IL23A and TNF-Alpha

Table 2A below provides exemplary compounds that bind to both IL23A and TNF-alpha that were utilized in the Examples below. These compounds were produced by recombinant methods known in the art (see, e.g., PCT Publications WO 2006/113665, WO 2008/157379, and WO 2010/080538, all of which are incorporated herein by reference). Briefly, plasmids encoding the first and second polypeptide for each compound were transfected together into CHO-S cells using FreeStyle MAX Reagent (CHO). The cells were cultured for 13-14 days and the compounds produced by the cells were purified using Protein-A chromatography. The compounds were further purified using a size exclusion chromatography.

TABLE 2A
Exemplary IL23A and TNF-alpha binding compounds

| | Large | Large | Small | Small | | | |
|---|---|---|---|---|---|---|---|
| | Chain | Chain | Chain | Chain | Linker | | SEQ ID NO: |
| Compound ID | vL | vH | vL | vH | types | Isotype | (1st/2nd) |
| Compound A | TNFa(1) | IL23A(1) | IL23A(1) | TNFa(1) | GS | IgG1KO- | 13/14 |
| | VL (SEQ | VH | VL (SEQ | VH (SEQ | | YTE | |
| | ID NO: | (SEQ ID | ID NO: 8) | ID NO: 1) | | | |
| | 2) | NO: 7) | | | | | |
| Compound B | TNFa(1) | IL23A(1) | IL23A(1) | TNFa(1) | VF | IgG1KO- | 15/16 |
| | VL (SEQ | VH | VL (SEQ | VH (SEQ | | YTE | |
| | ID NO: | (SEQ ID | ID NO: 8) | ID NO: 1) | | | |
| | 2) | NO: 7) | | | | | |
| Compound C | IL23A(1) | TNFa(1) | TNFa(1) | IL23A(1) | GS | IgG1KO- | 17/18 |
| | VL (SEQ | VH (SEQ | VL (SEQ | VH | | YTE | |
| | ID NO: | ID NO: | ID NO: 2) | (SEQ ID | | | |
| | 8) | 1) | | NO: 7) | | | |
| Compound D | IL23A(1) | TNFa(1) | TNFa(1) | IL23A(1) | VF | IgG1KO- | 19/20 |
| | VL (SEQ | VH (SEQ | VL (SEQ | VH | | YTE | |
| | ID NO: | ID NO: | ID NO: 2) | (SEQ ID | | | |
| | 8) | 1) | | NO: 7) | | | |
| Compound E | IL23A(1) | TNFa(2) | TNFa(2) | IL23A(1) | GS | IgG1KO- | 21/22 |
| | VL (SEQ | VH (SEQ | VL (SEQ | VH | | YTE | |
| | ID NO: | ID NO: | ID NO: 4) | (SEQ ID | | | |
| | 8) | 3) | | NO: 7) | | | |
| Compound F | IL23A(1) | TNFa(2) | TNFa(2) | IL23A(1) | VF | IgG1KO- | 23/24 |
| | VL (SEQ | VH (SEQ | VL (SEQ | VH | | YTE | |
| | ID NO: | ID NO: | ID NO: 4) | (SEQ ID | | | |
| | 8) | 3) | | NO: 7) | | | |
| Compound G | TNFa(2) | IL23A(1) | IL23A(1) | TNFa(2) | GS | IgG1KO- | 25/26 |
| | VL (SEQ | VH | VL (SEQ | VH (SEQ | | YTE | |
| | ID NO: | (SEQ ID | ID NO: 8) | ID NO: 3) | | | |
| | 4) | NO: 7) | | | | | |
| Compound H | TNFa(2) | IL23A(1) | IL23A(1) | TNFa(2) | VF | IgG1KO- | 27/28 |
| | VL (SEQ | VH | VL (SEQ | VH (SEQ | | YTE | |
| | ID NO: | (SEQ ID | ID NO: 8) | ID NO: 3) | | | |
| | 4) | NO: 7) | | | | | |
| Compound I | TNFa(3) | IL23A(1) | IL23A(1) | TNFa(3) | GS | IgG1KO- | 29/30 |
| | VL (SEQ | VH | VL (SEQ | VH (SEQ | | YTE | |
| | ID NO: | (SEQ ID | ID NO: 8) | ID NO: 5) | | | |
| | 6) | NO: 7) | | | | | |
| Compound J | IL23A(1) | TNFa(3) | TNFa(3) | IL23A(1) | GS | IgG1KO- | 31/32 |
| | VL (SEQ | VH (SEQ | VL (SEQ | VH | | YTE | |
| | ID NO: | ID NO: | ID NO: 6) | (SEQ ID | | | |
| | 8) | 5) | | NO: 7) | | | |
| Compound K | TNFa(3) | IL23A(1) | IL23A(1) | TNFa(3) | VF | IgG1KO- | 33/34 |
| | VL (SEQ | VH | VL (SEQ | VH (SEQ | | YTE | |
| | ID NO: | (SEQ ID | ID NO: 8) | ID NO: 5) | | | |
| | 6) | NO: 7) | | | | | |
| Compound L | IL23A(1) | TNFa(3) | TNFa(3) | IL23A(1) | VF | IgG1KO- | 35/36 |
| | VL (SEQ | VH (SEQ | VL (SEQ | VH | | YTE | |
| | ID NO: | ID NO: | ID NO: 6) | (SEQ ID | | | |
| | 8) | 5) | | NO: 7) | | | |
| Compound M | TNFa(1) | IL23A(1) | IL23A(1) | TNFa(1) | GS | IgG1KO | 44/45 |
| | VL (SEQ | VH | VL (SEQ | VH (SEQ | | | |
| | ID NO: | (SEQ ID | ID NO: 8) | ID NO: 1) | | | |
| | 2) | NO: 7) | | | | | |
| Compound N | IL23A(1) | TNFa(1) | TNFa(1) | IL23A(1) | VF | IgG1KO | 46/47 |
| | VL (SEQ | VH (SEQ | VL (SEQ | VH | | | |
| | ID NO: | ID NO: | ID NO: 2) | (SEQ ID | | | |
| | 8) | 1) | | NO: 7) | | | |
| Compound O | IL23A(1) | TNFa(2) | TNFa(2) | IL23A(1) | GS | IgG1KO | 48/49 |
| | VL (SEQ | VH (SEQ | VL (SEQ | VH | | | |
| | ID NO: | ID NO: | ID NO: 4) | (SEQ ID | | | |
| | 8) | 3) | | NO: 7) | | | |
| Compound P | IL23A(1) | TNFa(2) | TNFa(2) | IL23A(1) | VF | IgG1KO | 50/51 |
| | VL (SEQ | VH (SEQ | VL (SEQ | VH | | | |
| | ID NO: | ID NO: | ID NO: 4) | (SEQ ID | | | |
| | 8) | 3) | | NO: 7) | | | |
| Compound Q | TNFa(2) | IL23A(1) | IL23A(1) | TNFa(2) | GS | IgG4Pro | 52/53 |
| | VL (SEQ | VH | VL (SEQ | VH (SEQ | | | |
| | ID NO: | (SEQ ID | ID NO: 8) | ID NO: 3) | | | |
| | 4) | NO: 7) | | | | | |
| Compound R | TNFa(2) | IL23A(1) | IL23A(1) | TNFa(2) | GS | IgG4Pro- | 54/55 |
| | VL (SEQ | VH | VL (SEQ | VH (SEQ | | YTE | |
| | ID NO: | (SEQ ID | ID NO: 8) | ID NO: 3) | | | |
| | 4) | NO: 7) | | | | | |
| Compound S | TNFa(2) | IL23A(1) | IL23A(1) | TNFa(2) | GS | IgG4Pro | 56/57 |
| | VL (SEQ | VH | VL (SEQ | VH (SEQ | | | |
| | ID NO: | (SEQ ID | ID NO: 8) | ID NO: 3) | | | |
| | 4) | NO: 7) | | | | | |
| Compound T | TNFa(2) | IL23A(1) | IL23A(1) | TNFa(2) | GS | IgG4Pro- | 58/59 |
| | VL (SEQ | VH | VL (SEQ | VH (SEQ | | YTE | |
| | ID NO: | (SEQ ID | ID NO: 8) | ID NO: 3) | | | |
| | 4) | NO: 7) | | | | | |
| Compound U | TNFa(2) | IL23A(1) | IL23A(1) | TNFa(2) | VF | IgG1KO | 60/61 |
| | VL (SEQ | VH | VL (SEQ | VH (SEQ | | | |
| | ID NO: | (SEQ ID | ID NO: 8) | ID NO: 3) | | | |
| | 4) | NO: 7) | | | | | |
| Compound V | TNFa(2) | IL23A(1) | IL23A(1) | TNFa(2) | VF | IgG1WT | 62/63 |
| | VL (SEQ | VH | VL (SEQ | VH (SEQ | | | |
| | ID NO: | (SEQ ID | ID NO: 8) | ID NO: 3) | | | |
| | 4) | NO: 7) | | | | | |
| Compound W | TNFa(2) | IL23A(1) | IL23A(1) | TNFa(2) | VF | IgG4Pro | 64/65 |
| | VL (SEQ | VH | VL (SEQ | VH (SEQ | | | |
| | ID NO: | (SEQ ID | ID NO: 8) | ID NO: 3) | | | |
| | 4) | NO: 7) | | | | | |
| Compound X | TNFa(2) | IL23A(1) | IL23A(1) | TNFa(2) | VF | IgG4Pro- | 66/67 |
| | VL (SEQ | VH | VL (SEQ | VH (SEQ | | YTE | |
| | ID NO: | (SEQ ID | ID NO: 8) | ID NO: 3) | | | |
| | 4) | NO: 7) | | | | | |
| Compound Y | IL23A(1) | TNFa(2) | TNFa(2) | IL23A(1) | GS | IgG1WT | 68/69 |
| | VL (SEQ | VH (SEQ | VL (SEQ | VH | | | |
| | ID NO: | ID NO: | ID NO: 4) | (SEQ ID | | | |
| | 8) | 3) | | NO: 7) | | | |
| Compound Z | IL23A(1) | TNFa(2) | TNFa(2) | IL23A(1) | GS | IgG4Pro | 70/71 |
| | VL (SEQ | VH (SEQ | VL (SEQ | VH | | | |
| | ID NO: | ID NO: | ID NO: 4) | (SEQ ID | | | |
| | 8) | 3) | | NO: 7) | | | |
| Compound AA | IL23A(1) | TNFa(2) | TNFa(2) | IL23A(1) | GS | IgG4Pro- | 72/73 |
| | VL (SEQ | VH (SEQ | VL (SEQ | VH | | YTE | |
| | ID NO: | ID NO: | ID NO: 4) | (SEQ ID | | | |
| | 8) | 3) | | NO: 7) | | | |
| Compound AB | IL23A(1) | TNFa(2) | TNFa(2) | IL23A(1) | VF | IgG1WT | 74/75 |
| | VL (SEQ | VH (SEQ | VL (SEQ | VH | | | |
| | ID NO: | ID NO: | ID NO: 4) | (SEQ ID | | | |
| | 8) | 3) | | NO: 7) | | | |
| Compound AC | IL23A(1) | TNFa(2) | TNFa(2) | IL23A(1) | VF | IgG4Pro | 76/77 |
| | VL (SEQ | VH (SEQ | VL (SEQ | VH | | | |
| | ID NO: | ID NO: | ID NO: 4) | (SEQ ID | | | |
| | 8) | 3) | | NO: 7) | | | |
| Compound AD | IL23A(1) | TNFa(2) | TNFa(2) | IL23A(1) | VF | IgG4Pro- | 78/79 |
| | VL (SEQ | VH (SEQ | VL (SEQ | VH | | YTE | |
| | ID NO: | ID NO: | ID NO: 4) | (SEQ ID | | | |
| | 8) | 3) | | NO: 7) | | | |
| Compound AE | TNFa(3) | IL23A(1) | IL23A(1) | TNFa(3) | GS | IgG1KO | 80/81 |
| | VL (SEQ | VH | VL (SEQ | VH (SEQ | | | |
| | ID NO: | (SEQ ID | ID NO: 8) | ID NO: 5) | | | |
| | 6) | NO: 7) | | | | | |
| Compound AF | TNFa(3) | IL23A(1) | IL23A(1) | TNFa(3) | GS | IgG1WT | 82/83 |
| | VL (SEQ | VH | VL (SEQ | VH (SEQ | | | |
| | ID NO: | (SEQ ID | ID NO: 8) | ID NO: 5) | | | |
| | 6) | NO: 7) | | | | | |
| Compound AG | TNFa(3) | IL23A(1) | IL23A(1) | TNFa(3) | GS | IgG4Pro | 84/85 |
| | VL (SEQ | VH | VL (SEQ | VH (SEQ | | | |
| | ID NO: | (SEQ ID | ID NO: 8) | ID NO: 5) | | | |
| | 6) | NO: 7) | | | | | |
| Compound AH | TNFa(3) | IL23A(1) | IL23A(1) | TNFa(3) | GS | IgG4Pro- | 86/87 |
| | VL (SEQ | VH | VL (SEQ | VH (SEQ | | YTE | |
| | ID NO: | (SEQ ID | ID NO: 8) | ID NO: 5) | | | |
| | 6) | NO: 7) | | | | | |
| Compound Al | TNFa(3) | IL23A(1) | IL23A(1) | TNFa(3) | VF | IgG1KO | 88/89 |
| | VL (SEQ | VH | VL (SEQ | VH (SEQ | | | |
| | ID NO: | (SEQ ID | ID NO: 8) | ID NO: 5) | | | |
| | 6) | NO: 7) | | | | | |
| Compound AJ | TNFa(3) | IL23A(1) | IL23A(1) | TNFa(3) | VF | IgG1WT | 90/91 |
| | VL (SEQ | VH | VL (SEQ | VH (SEQ | | | |
| | ID NO: | (SEQ ID | ID NO: 8) | ID NO: 5) | | | |
| | 6) | NO: 7) | | | | | |
| Compound AK | TNFa(3) | IL23A(1) | IL23A(1) | TNFa(3) | VF | IgG4Pro | 92/93 |
| | VL (SEQ | VH | VL (SEQ | VH (SEQ | | | |
| | ID NO: | (SEQ ID | ID NO: 8) | ID NO: 5) | | | |
| | 6) | NO: 7) | | | | | |
| Compound AL | TNFa(3) | IL23A(1) | IL23A(1) | TNFa(3) | VF | IgG4Pro- | 94/95 |
| | VL (SEQ | VH | VL (SEQ | VH (SEQ | | YTE | |
| | ID NO: | (SEQ ID | ID NO: 8) | ID NO: 5) | | | |
| | 6) | NO: 7) | | | | | |
| Compound | IL23A(1) | TNFa(3) | TNFa(3) | IL23A(1) | GS | IgG1KO | 96/97 |
| AM | VL (SEQ | VH (SEQ | VL (SEQ | VH | | | |
| | ID NO: | ID NO: | ID NO: 6) | (SEQ ID | | | |
| | 8) | 5) | | NO: 7) | | | |
| Compound AN | IL23A(1) | TNFa(3) | TNFa(3) | IL23A(1) | GS | IgG1WT | 98/99 |
| | VL (SEQ | VH (SEQ | VL (SEQ | VH | | | |
| | ID NO: | ID NO: | ID NO: 6) | (SEQ ID | | | |
| | 8) | 5) | | NO: 7) | | | |
| Compound AO | IL23A(1) | TNFa(3) | TNFa(3) | IL23A(1) | GS | IgG4Pro | 100/101 |
| | VL (SEQ | VH (SEQ | VL (SEQ | VH | | | |
| | ID NO: | ID NO: | ID NO: 6) | (SEQ ID | | | |
| | 8) | 5) | | NO: 7) | | | |
| Compound AP | IL23A(1) | TNFa(3) | TNFa(3) | IL23A(1) | GS | IgG4Pro- | 102/103 |
| | VL (SEQ | VH (SEQ | VL (SEQ | VH | | YTE | |
| | ID NO: | ID NO: | ID NO: 6) | (SEQ ID | | | |
| | 8) | 5) | | NO: 7) | | | |
| Compound AQ | IL23A(1) | TNFa(3) | TNFa(3) | IL23A(1) | VF | IgG1KO | 104/105 |
| | VL (SEQ | VH (SEQ | VL (SEQ | VH | | | |
| | ID NO: | ID NO: | ID NO: 6) | (SEQ ID | | | |
| | 8) | 5) | | NO: 7) | | | |
| Compound AR | IL23A(1) | TNFa(3) | TNFa(3) | IL23A(1) | VF | IgG1WT | 106/107 |
| | VL (SEQ | VH (SEQ | VL (SEQ | VH | | | |
| | ID NO: | ID NO: | ID NO: 6) | (SEQ ID | | | |
| | 8) | 5) | | NO: 7) | | | |
| Compound AS | IL23A(1) | TNFa(3) | TNFa(3) | IL23A(1) | VF | IgG4Pro | 108/109 |
| | VL (SEQ | VH (SEQ | VL (SEQ | VH | | | |
| | ID NO: | ID NO: | ID NO: 6) | (SEQ ID | | | |
| | 8) | 5) | | NO: 7) | | | |
| Compound AT | IL23A(1) | TNFa(3) | TNFa(3) | IL23A(1) | VF | IgG4Pro- | 110/111 |
| | VL (SEQ | VH (SEQ | VL (SEQ | VH | | YTE | |
| | ID NO: | ID NO: | ID NO: 6) | (SEQ ID | | | |
| | 8) | 5) | | NO: 7) | | | |
| Compound AU | TNFa(2) | IL23A(1) | IL23A(1) | TNFa(2) | GS | IgG1KO | 112/113 |
| | VL (SEQ | VH | VL (SEQ | VH (SEQ | | | |
| | ID NO: | (SEQ ID | ID NO: 8) | ID NO: 3) | | | |
| | 4) | NO: 7) | | | | | |
| Compound AV | TNFa(1) | IL23A(1) | IL23A(1) | TNFa(1) | GS | IgG1WT | 114/115 |
| | VL (SEQ | VH | VL (SEQ | VH (SEQ | | | |
| | ID NO: | (SEQ ID | ID NO: 8) | ID NO: 1) | | | |
| | 2) | NO: 7) | | | | | |
| Compound | TNFa(1) | IL23A(1) | IL23A(1) | TNFa(1) | GS | IgG4Pro | 116/117 |
| AW | VL (SEQ | VH | VL (SEQ | VH (SEQ | | | |
| | ID NO: | (SEQ ID | ID NO: 8) | ID NO: 1) | | | |
| | 2) | NO: 7) | | | | | |
| Compound AX | TNFa(1) | IL23A(1) | IL23A(1) | TNFa(1) | GS | IgG4Pro- | 118/119 |
| | VL (SEQ | VH | VL (SEQ | VH (SEQ | | YTE | |
| | ID NO: | (SEQ ID | ID NO: 8) | ID NO: 1) | | | |
| | 2) | NO: 7) | | | | | |
| Compound AY | TNFa(1) | IL23A(1) | IL23A(1) | TNFa(1) | VF | IgG1KO | 120/121 |
| | VL (SEQ | VH | VL (SEQ | VH (SEQ | | | |
| | ID NO: | (SEQ ID | ID NO: 8) | ID NO: 1) | | | |
| | 2) | NO: 7) | | | | | |
| Compound AZ | TNFa(1) | IL23A(1) | IL23A(1) | TNFa(1) | VF | IgG1WT | 122/123 |
| | VL (SEQ | VH | VL (SEQ | VH (SEQ | | | |
| | ID NO: | (SEQ ID | ID NO: 8) | ID NO: 1) | | | |
| | 2 | NO: 7) | | | | | |
| Compound BA | TNFa(1) | IL23A(1) | IL23A(1) | TNFa(1) | VF | IgG4Pro | 124/125 |
| | VL (SEQ | VH | VL (SEQ | VH (SEQ | | | |
| | ID NO: | (SEQ ID | ID NO: 8) | ID NO: 1) | | | |
| | 2) | NO: 7) | | | | | |
| Compound BB | TNFa(1) | IL23A(1) | IL23A(1) | TNFa(1) | VF | IgG4Pro- | 126/127 |
| | VL (SEQ | VH | VL (SEQ | VH (SEQ | | | |
| | ID NO: | (SEQ ID | ID NO: 8) | ID NO: 1) | | YTE | |
| | 2) | NO: 7) | | | | | |
| Compound BC | IL23A(1) | TNFa(1) | TNFa(1) | IL23A(1) | GS | IgG1KO | 128/129 |
| | VL (SEQ | VH (SEQ | VL (SEQ | VH | | | |
| | ID NO: | ID NO: | ID NO: 2) | (SEQ ID | | | |
| | 8) | 1) | | NO: 7) | | | |
| Compound BD | IL23A(1) | TNFa(1) | TNFa(1) | IL23A(1) | GS | IgG1WT | 130/131 |
| | VL (SEQ | VH (SEQ | VL (SEQ | VH | | | |
| | ID NO: | ID NO: | ID NO: 2) | (SEQ ID | | | |
| | 8) | 1) | | NO: 7) | | | |
| Compound BE | IL23A(1) | TNFa(1) | TNFa(1) | IL23A(1) | GS | IgG4Pro | 132/133 |
| | VL (SEQ | VH (SEQ | VL (SEQ | VH | | | |
| | ID NO: | ID NO: | ID NO: 2) | (SEQ ID | | | |
| | 8) | 1) | | NO: 7) | | | |
| Compound BF | IL23A(1) | TNFa(1) | TNFa(1) | IL23A(1) | GS | IgG4Pro- | 134/135 |
| | VL (SEQ | VH (SEQ | VL (SEQ | VH | | YTE | |
| | ID NO: | ID NO: | ID NO: 2) | (SEQ ID | | | |
| | 8) | 1) | | NO: 7) | | | |
| Compound BG | TNFa(2) | IL23A(1) | IL23A(1) | TNFa(2) | GS | IgG1WT | 136/137 |
| | VL (SEQ | VH | VL (SEQ | VH (SEQ | | | |
| | ID NO: | (SEQ ID | ID NO: 8) | ID NO: 3) | | | |
| | 4) | NO: 7) | | | | | |
| Compound BH | IL23A(1) | TNFa(1) | TNFa(1) | IL23A(1) | VF | IgG1WT | 138/139 |
| | VL (SEQ | VH (SEQ | VL (SEQ | VH | | | |
| | ID NO: | ID NO: | ID NO: 2) | (SEQ ID | | | |
| | 8) | 1) | | NO: 7) | | | |
| Compound BI | IL23A(1) | TNFa(1) | TNFa(1) | IL23A(1) | VF | IgG4Pro | 140/141 |
| | VL (SEQ | VH (SEQ | VL (SEQ | VH | | | |
| | ID NO: | ID NO: | ID NO: 2) | (SEQ ID | | | |
| | 8) | 1) | | NO: 7) | | | |
| Compound BJ | IL23A(1) | TNFa(1) | TNFa(1) | IL23A(1) | VF | IgG4Pro- | 142/143 |
| | VL (SEQ | VH (SEQ | VL (SEQ | VH | | YTE | |
| | ID NO: | ID NO: | ID NO: 2) | (SEQ ID | | | |
| | 8) | 1) | | NO: 7) | | | |

TNFa = TNF-alpha, VL = variable domain light chain, VH = variable domain heavy chain, GS = GGGSGGGG (SEQ ID NO: 9), LGGGSG (SEQ ID NO: 10), or both, VF = FNRGES (SEQ ID NO: 11), VEPKSS (SEQ ID NO: 12), or both, IgG1WT = IgG1 wild type; IgG1KO-YTE = IgG1 with a M252Y/S254T/T256E triple mutation in the Fc region and also comprising L234A/L235A mutations, IgG4Pro-YTE = IgG4 with a M252Y/S254T/T256E triple mutation in the Fc region and also comprising S241P mutation, IgG1KO = truncated Fc region comprising L234A/L235A mutations, IgG4Pro = comprising S241P mutation. 1^{st} = first polypeptide, 2nd = second polypeptide. The numbering of mutations is Kabat numbering for a conventional antibody starting with the antibody convention at CH1.

The below control antibodies were also used for comparison purposes. The controls were monoclonal antibodies that targeted either TNFa or 1123.

TABLE 2B

| Control compounds | VH sequence | VL sequence |
|---|---|---|
| Control antibody 1 | TNFa(1) VH | TNFa(1) VL |
| (TNFa monoclonal | (SEQ ID | (SEQ ID |
| antibody) | NO: 1) | NO: 2) |
| Control antibody 2 | TNFa(2) VH | TNFa(2) VL |
| (TNFa monoclonal | (SEQ ID | (SEQ ID |
| antibody) | NO: 3) | NO: 4) |
| Control Antibody 3 | IL23A(1) VH | IL23A(1) VL |
| (IL23 monoclonal | (SEQ ID | (SEQ ID |
| antibody) | NO: 7) | NO: 8) |

### Example 2. Surface Plasmon Resonance (SPR) Affinity of Exemplary Compounds

Test compounds were analyzed by SPR to determine affinity for TNF-alpha and IL23A.

### Materials and Methods

SPR experiments were performed on a ProteOn XPR36 instrument (Bio Rad). A GLM chip was preconditioned with sequential injections of 60 sec of 0.5% SDS, 50 mM NaOH, and 100 mM HCl at a flow rate of 30 μl/min both vertical and horizontal directions. The preconditioned GLM chip was then activated by an injection of EDC (76.7 mg/ml) and sulfo-NHS (21.7 mg/ml) mixture with ratio of 1:1 in 6 horizontal channels. Goat-anti-human IgG (GAHA) Fc gamma (Invitrogen) at a concentration of 30 μg/ml in 10 mM, pH 5.0 sodium acetate buffer was immobilized to 8,000 resonance units on the activated GLM chip in 6 horizontal channels. The chip was finally deactivated with 1 M ethanolamine HCl in 6 horizontal channels. The prepared GAHA chip was rotated to vertical direction to capture test compounds, over 5 vertical channels and the last channel was used as a column reference. The captured chip was then rotated again to the horizontal direction for binding. Linked human IL-23 (Boehringer Ingelheim Pharmaceuticals, Inc) with five concentrations, 10.0 nM, 5.00 nM, 2.50 nM, 1.25 nM and 0.625 nM, were injected horizontally over the test compound surfaces for 10 minutes at a flow rate of 40 μl/min in the following running buffer (Bio Rad): phosphate buffer saline (pH 7.4), 0.005% Tween 20. The dissociation was allowed for 2 hour. The GAHA surface was regenerated using short pulse injection (18 seconds) of 0.85% phosphoric acid (Bio Rad) at a flow rate of 100 μl/min both horizontal and vertical directions after 10 min association and 2 hr dissociation. The regenerated GAHA was ready for another binding cycle. Binding of compounds to human TNF-alpha or cynomolgous TNF-alpha was done in similar way.

### Results

The results in Table 3 show that both compounds tested were able to bind TNF-alpha and IL23 with a dissociation constant (KD) in the picomolar range.

TABLE 3

| | KD to human | KD to | |
|---|---|---|---|
| | TNF-alpha | cynomologus | KD to human |
| Compound ID | (pM) | TNF-alpha (pM) | IL23 (pM) |
| Compound A | 2.14 | 7.71 | 4.28 ± 2.03 |
| Compound E | 4.11 ± 0.68 | 37.1 ± 16.2 | 7.00 ± 6.92 |

### Example 3. Flow Cytometry Assessment of Binding to Membrane Bound TNF-Alpha

Test compounds were assessed for their ability to dose dependently bind to cell lines transfected to express membrane bound TNF-alpha.

### Material and Methods

All reagents were prepared in flow cytometry staining buffer (BioLegend). Membrane expressed TNF-alpha transfected cell lines (Jurkat and CHO) and parental cell lines were harvested from tissue culture vessels, washed, counted and resuspended to 1×10\hat{ }6 cells/ml in flow cytometry staining buffer. One hundred microliters of the cell suspension was added to 96 well microtiter plates and placed on ice. Titrations of test compounds were prepared and 50 uL was added to the cells. After sixty minute incubation on ice, the cell+test compounds were washed and 50 uL of a secondary antibody (Jackson ImmunoResearch) was added. The samples were incubated in the dark, at 4 C, for 60 minutes, followed by washes. After a final wash the cells were resuspended in 60 uL of fixative (BD Bioscience). Median fluorescence was determined for each sample in a flow cytometer and plotted versus the concentration of the test sample. EC₅₀ values were calculated using the 4 Parameter Logistic enabled by the Excel add-in XLfit (Activity Base software, ID Business Solutions, Ltd.). The EC₅₀ values shown below are Geomeans calculated across multiple experiments for each test sample and are shown in Table 4.

### Results

The results shown in Table 4 below demonstrate that the compounds tested bound to membrane bound TNF-alpha in a dose dependent manner.

TABLE 4
EC50 values for membrane bound TNF-alpha.

| | mTNF-Jurkat Cell | mTNF-CHO Cell |
|---|---|---|
| | Binding EC₅₀ PM | Binding EC₅₀ PM |
| Compound ID | (Geomean) | (Geomean) |
| Compound M | 650 | 950 |
| Compound A | 910 | 890 |
| Compound O | 270 | 770 |
| Compound E | 200 | 450 |
| Control antibody 1 | 310 | 400 |
| (TNFa) | | |
| Control antibody 2 | 230 | 310 |
| (TNFa) | | |

### Example 4. In Vitro L929 Cytotoxicity Assay

The compounds were tested for their ability to inhibit TNF-alpha induced cytotoxicity.

### Methods and Materials

This protocol used the PrestoBlue™0 Cell Viability Reagent to determine cytotoxicity of recombinant human TNF-alpha. A more detailed protocol for the PrestoBlue Cell Viability Protocol can be downloaded from the Invitrogen website (Invitrogen.com). L929 cells were grown and harvested. 1.5×10⁴ cells were transferred to each well of a 96-well plate for incubated overnight at 37° C. Serial dilutions of compounds were prepared starting at 5 nM in complete assay medium containing 10 μg/ml of actinomycin D and 1000 μg/ml of rhTNF-alpha. The positive controls contained 20 ng/ml rhTNF-alpha and 1 μg/ml actinomycin D. The negative control contained no TNF-alpha. 10 μL of the dilutions was added to corresponding wells and incubated overnight at 37° C. in a 5% CO2. PrestoBlue™ reagent was added to wells and the plate was incubated for 2 hour at 37° C. in a 5% CO2. The relative fluorescence unit of each well was measured using a Victor™×2 plate reader (excitation: 560 nm, emission: 590 nm). The fluorescent units (Y-axis) versus concentration of test compound (X-axis) were plotted and the IC₅₀ and IC₉₀ values of test compounds were calculated by using Graphpad software.

### Results

The results in Table 5 show that the tested compounds were able to inhibit TNF-alpha induced cytotoxicity in a dose-dependent manner.

TABLE 5

| | | L929 TNF |
|---|---|---|
| | IC₅₀ | Cytotox pM |
| Compound ID | IC₉₀ | Geomean |
| Compound M | IC₅₀ | 19 |
| Compound M | IC₉₀ | 55 |
| Compound A | IC₅₀ | 20 |
| Compound A | IC₉₀ | 67 |
| Compound N | IC₅₀ | 34 |
| Compound N | IC₉₀ | |
| Compound D | IC₅₀ | |
| Compound D | IC₉₀ | |
| Compound O | IC₅₀ | 4.2 |
| Compound O | IC₉₀ | 16 |
| Compound E | IC₅₀ | 4.1 |
| Compound E | IC₉₀ | 17 |
| Compound P | IC₅₀ | 3.4 |
| Compound P | IC₉₀ | 14 |
| Compound F | IC₅₀ | 2.5 |
| Compound F | IC₉₀ | 10 |
| Control antibody 1 | IC₅₀ | 62 |
| (TNFa) | | |
| Control antibody 1 | IC₉₀ | 230 |
| (TNFa) | | |
| Control antibody 2 | IC₅₀ | 20 |
| (TNFa) | | |
| Control antibody 2 | IC₉₀ | 95 |
| (TNFa) | | |

### Example 5. Inhibition of TNF-Alpha Dependent IL-8 Release in HeLa Cells

Anti-TNF test samples were assessed for their ability to inhibit the TNF dependent release of IL8 from the human cell line, HeLa. The samples were tested against a high and low concentration of recombinant human TNF-alpha and a single (high) concentration of recombinant cynomolgus TNF-alpha.

### Materials and Methods

Briefly, HeLa cells (ATCC) were harvested, washed, counted and resuspended to 4×10\hat{ }5 cells/ml in a standard complete media of (v/v) 10% Fetal Bovine Serum with 1% Penicillin &Streptomycin (CM). One hundred microliters of the HeLa cell suspension was added to 96 well microtiter plates. Recombinant human TNF-alpha (R&D Systems) at two concentrations (147 nM or 4.4 nM) as well as generated recombinant cynomolgus TNF-alpha (Boehringer Ingelheim Pharmaceuticals, Inc.) (147 nM) were pre-incubated for 30 minutes at 37 C with CM alone or with titrations of test samples. After the pre-incubation of test sample+TNF-alpha, 100 ul of the mixture(s) was added to the cells and the test plates were incubated at 37 C with 5% CO₂-humidified air for 20 hours. Control samples received either CM (unstimulated controls) or recombinant TNF-alpha diluted in CM (stimulated controls).

After the incubation, supernatants were assayed for IL8 in an ELISA kit (MesoScale Discovery) following the manufacturer's instructions. Interpolated IL8 pg/ml values were determined for each sample and converted to percent of control (POC). The POC was plotted versus concentration of the test sample and IC₅₀ and IC₉₀ values were calculated using a 4 Parameter Logistic Model enabled by the Excel add-in XLfit (Activity Base software, ID Business Solutions, Ltd.).

The test compounds were analyzed with respect to the IC₅₀/IC₉₀ as described above, and Geomeans were calculated across multiple experiments for each test sample and shown in Table 6.

### Results

The results in Tables 6 show that the IC₅₀ and IC₉₀ Geomean values for the tested compounds were similar to the IC₅₀ and IC₉₀ Geomean values for Control Antibody 1 and Control antibody 2. The data demonstrates that the test compounds dose dependently inhibited the TNF-alpha induced IL-8 secretion with either human (at two concentrations tested) or cyno recombinant TNF-alpha.

TABLE 6

| | | HeLa IL8 | HeLa IL8 Hi- | HeLa IL8 |
|---|---|---|---|---|
| | IC₅₀ | Lo-Hu-TNF | Hu-TNF pM | Cyno-TNF pM |
| Compound ID | IC₉₀ | pM Geomean | Geomean | Geomean |
| Compound M | IC₅₀ | 7.9 | 260 | 150 |
| Compound M | IC₉₀ | 48 | 420 | 270 |
| Compound A | IC₅₀ | 8 | 280 | 120 |
| Compound A | IC₉₀ | 41 | 460 | 260 |
| Compound N | IC₅₀ | 9.2 | 350 | 170 |
| Compound N | IC₉₀ | 54 | 570 | 330 |
| Compound D | IC₅₀ | 11 | 380 | 190 |
| Compound D | IC₉₀ | 63 | 590 | 390 |
| Compound O | IC₅₀ | 9.9 | 430 | 300 |
| Compound O | IC₉₀ | 43 | 760 | 970 |
| Compound E | IC₅₀ | 9.2 | 320 | 180 |
| Compound E | IC₉₀ | 35 | 530 | 600 |
| Compound P | IC₅₀ | 9.2 | 410 | 210 |
| Compound P | IC₉₀ | 36 | 810 | 810 |
| Compound F | IC₅₀ | 7.9 | 350 | 190 |
| Compound F | IC₉₀ | 39 | 660 | 740 |
| Control antibody 1 | IC₅₀ | 34 | 330 | 170 |
| (TNFa) | | | | |
| Control antibody 1 | IC₉₀ | 140 | 490 | 330 |
| (TNFa) | | | | |
| Control antibody 2 | IC₅₀ | 11 | 290 | 280 |
| (TNFa) | | | | |
| Control antibody 2 | IC₉₀ | 55 | 520 | 1200 |
| (TNFa) | | | | |

### Example 6. Inhibition of TNF-Alpha Dependent 118 in Whole Blood

TNF is a potent inducer of IL8 release from human cells. Compounds were tested for their ability to inhibit TNF-alpha induced IL-8 release in whole blood samples.

### Methods and Materials

Briefly 120 μL of heparinized human whole blood was added to each well in a 96 well microtiter plate. Assay reagents were prepared in a standard T cell media (TCM). Titrations of test samples were prepared at 10× concentrations and pre-incubated with a 10× concentration of human recombinant TNF (100 ng/ml, R&D Systems) for 1 hour at 37 C. After this pre-incubation, 30 ul of the cytokine/test compound mixture was added to the whole blood along with 30 μL of appropriate controls in TCM and incubated at 37 C with 5% CO₂-humidified air for 48 hours. Control samples received either TCM (unstimulated controls) or recombinant human TNF-alpha diluted in TCM (stimulated controls). After the incubation, supernatants were assayed for IL8 in an ELISA kit (MesoScale Discovery) following manufacturer's instructions. Interpolated IL8 pg/ml values were determined for each sample and converted to percent of control (POC). The POC was plotted versus concentration of the test sample and IC₅₀ and IC₉₀ values were calculated using a 4 Parameter Logistic Model enabled by the Excel add-in XLfit (Activity Base software, ID Business Solutions, Ltd.).

The test compounds were analyzed with respect to the IC₅₀/IC₉₀ as described above, and Geomeans were calculated across multiple experiments for each test sample and shown in Table 7.

### Results

The results in Table 7 show that the IC₅₀ and IC₉₀ Geomean values for the tested compounds were similar to the IC₅₀ and IC₉₀ Geomean values for Control antibody 1 and control antibody 2. The data demonstrates that the test compounds dose dependently inhibited the TNF-alpha induced IL8 release in human whole blood.

TABLE 7

| | IC₅₀ | TNF-IL8 Whole |
|---|---|---|
| Compound ID | IC₉₀ | Blood pM Geomean |
| Compound M | IC₅₀ | 380 |
| Compound M | IC₉₀ | 790 |
| Compound A | IC₅₀ | 360 |
| Compound A | IC₉₀ | 490 |
| Compound N | IC₅₀ | 270 |
| Compound N | IC₉₀ | 520 |
| Compound D | IC₅₀ | 560 |
| Compound D | IC₉₀ | 1100 |
| Compound O | IC₅₀ | 320 |
| Compound O | IC₉₀ | 470 |
| Compound E | IC₅₀ | 340 |
| Compound E | IC₉₀ | 610 |
| Compound P | IC₅₀ | 290 |
| Compound P | IC₉₀ | 420 |
| Compound F | IC₅₀ | 310 |
| Compound F | IC₉₀ | 450 |
| Control antibody 1 | IC₅₀ | 320 |
| (TNFa) | | |
| Control antibody 1 | IC₉₀ | 490 |
| (TNFa) | | |
| Control antibody 2 | IC₅₀ | 330 |
| (TNFa) | | |
| Control antibody 2 | IC₉₀ | 600 |
| (TNFa) | | |

### Example 7. NF-kappaB and STAT3 Phosphorylation Assays

IL23 engagement with its heterodimeric receptor complex (IL12Rβ1-IL23R) results in the downstream phosphorylation of Signal transducer and activator of transcription 3 (STAT3). TNF engagement with its receptors (TNFR1/TNFR2) results in the downstream phosphorylation of nuclear factor of kappa light polypeptide gene enhancer in B-cells (NF-κB). Compounds were assessed for their ability to inhibit TNF-dependent phosphorylation of NF-κB in Jurkat cells, and IL23-dependent phosphorylation of STAT3 in DB cells.

### Methods and Materials

Briefly, cultures of Jurkat cells (ATCC) and DB cells (ATCC) growing in log phase were harvested, washed, counted and resuspended to 2×10\hat{ }7 cells/mL in a standard complete media (CM; RPMI1640 with (v/v) 10% FCS and 1× Penicillin-Streptomycin (Invitrogen)). Titrations of test samples were prepared at 4× concentrations and pre-incubated with a mixture of 4× human recombinant IL23 (Boehringer Ingelheim Pharmaceuticals, Inc.) and recombinant human TNF (R&D Systems) for 1 hour at 37 C. After the pre-incubation of the test reagent+cytokine mixture, 100 μL of the mixture was added to wells containing 100 μL of cells in duplicate. Controls were setup as follows: 100 μL of the diluted TNF/IL23+100 μL combined cells (stimulated control), or 100 μL of CM+100 μL combined cells (unstimulated control). The assay plates were incubated for exactly 10 minutes at 37° C. with 5% CO₂-humidified air. After the incubation, cell lysates were prepared and p-NF-κB and p-STAT3 was assessed following the manufacturer's instructions (MesoScale Discovery). p-NF-κB and p-STAT-3 raw values were determined for each sample and converted to percent of control (POC). The POC was plotted (Y-axis) versus concentration of the test agent (X-axis). IC₅₀ and IC₉₀ values were calculated using the 4 Parameter Logistic Model enabled by the Excel add-in XLfit (Activity Base software, ID Business Solutions, Ltd.).

The test compounds were analyzed with respect to the IC₅₀/IC₉₀ as described above, and Geomeans were calculated across multiple experiments for each test sample and shown in Table 10. Note: this assay provides confidence that that the dual molecule is capable of neutralizing both downstream signaling events. The assay time point is optimal for the p-NF-κB signal only and therefore the calculated IC₅₀/IC₉₀ does not reflect the overall potencies in a quantitative manner.

### Results

The results in Table 8 show that the test compounds were able to inhibit both TNF-alpha induced NF-kB phosphorylation as well as IL23 induced phosphorylation of STAT3 in DB cells.

TABLE 8

| | | Dual | Dual |
|---|---|---|---|
| | | Phospho Jurkat- | Phospho DB- |
| | IC₅₀ | pNf-Kb pM | pSTAT3 pM |
| Compound ID | IC₉₀ | Geomean* | Geomean* |
| Compound M | IC₅₀ | 290 | 190 |
| Compound M | IC₉₀ | 680 | 580 |
| Compound A | IC₅₀ | 300 | 200 |
| Compound A | IC₉₀ | 480 | 500 |
| Compound N | IC₅₀ | 300 | 210 |
| Compound N | IC₉₀ | 620 | 760 |
| Compound D | IC₅₀ | 270 | 170 |
| Compound D | IC₉₀ | 810 | 560 |
| Compound O | IC₅₀ | 210 | 210 |
| Compound O | IC₉₀ | 740 | 580 |
| Compound E | IC₅₀ | 260 | 230 |
| Compound E | IC₉₀ | 340 | 770 |
| Compound P | IC₅₀ | 290 | 340 |
| Compound P | IC₉₀ | 340 | 630 |
| Compound F | IC₅₀ | 280 | 360 |
| Compound F | IC₉₀ | 760 | 980 |
| Control antibody 1 | IC₅₀ | 360 | NA |
| (TNFa) | | | |
| Control antibody 1 | IC₉₀ | 660 | NA |
| (TNFa) | | | |
| Control antibody 2 | IC₅₀ | 260 | NA |
| (TNFa) | | | |
| Control antibody 2 | IC₉₀ | 420 | NA |
| (TNFa) | | | |
| Control antibody | IC₅₀ | NA | 89 |
| 3(IL23A) | | | |
| Control antibody | IC₉₀ | NA | 230 |
| 3(IL23A) | | | |

*Results are semi-quantitative and optimized more to the TNF readout.
NA; No Activity

### Example 8. Inhibition of IL23 Induced STAT3 Phosphorylation in DB Cells

IL23 engagement with its heterodimeric receptor complex (IL12Rβ1-IL23R) results in the downstream phosphorylation of Signal transducer and activator of transcription 3 (STAT3). Anti-IL23 test samples were assessed for their ability to inhibit the IL23 dependent phosphorylation in the human DB cell line.

### Materials and Methods

Briefly 100 uL of the human DB cell line (ATCC) grown in log phase was added to each well in a 96 well microtiter plate at a concentration of 1×10\hat{ }7 cells/ml. Assay reagents were prepared in a complete media (CM; RPMI1640 with (v/v) 10% Fetal Calf Serum and 1× Penicillin-Streptomycin (Invitrogen)). Titrations of test samples were prepared at 4× concentrations and pre-incubated with a 4× concentration of human recombinant IL23 (Boehringer Ingelheim Pharmaceuticals, Inc.) for 1 hour at 37 C. After this pre-incubation, 100 ul of the cytokine/test sample mixture was added to the 100 uL of DB cells and incubated at 37 C with 5% CO₂-humidified air for 30 minutes. Control samples received either CM (unstimulated controls) or recombinant human IL23 diluted in CM (stimulated controls). After the incubation, cell lysates were prepared and pSTAT3 was assessed following the manufacturer's instructions (MesoScale Discovery). Raw pSTAT3 values were determined for each sample and converted to percent of control (POC). The POC was plotted versus concentration of the test sample and IC₅₀ and IC₉₀ values were calculated using a 4 Parameter Logistic Model enabled by the Excel add-in XLfit (Activity Base software, ID Business Solutions, Ltd.). The test compounds were analyzed with respect to the IC₅₀/IC₉₀ as described above, and Geomeans were calculated across multiple experiments for each test sample and shown in Table 9.

### Results

The results in Table 9 show that the IC₅₀ and IC₉₀ Geomean values for the tested compounds were similar to the IC₅₀ and IC₉₀ Geomean values for an anti-IL23Ap19 control antibody. The data demonstrates that the test compounds dose dependently inhibited the IL23 induced phosphorylation of STAT3 in DB cells.

TABLE 9

| | | hIL23 pSTAT3 |
|---|---|---|
| | IC₅₀ | DB assay pM |
| Compound ID | IC₉₀ | Geomean |
| Compound M | IC₅₀ | 190 |
| Compound M | IC₉₀ | 530 |
| Compound A | IC₅₀ | 210 |
| Compound A | IC₉₀ | 420 |
| Compound N | IC₅₀ | 240 |
| Compound N | IC₉₀ | 510 |
| Compound D | IC₅₀ | 280 |
| Compound D | IC₉₀ | 560 |
| Compound O | IC₅₀ | 300 |
| Compound O | IC₉₀ | 720 |
| Compound E | IC₅₀ | 300 |
| Compound E | IC₉₀ | 700 |
| Compound P | IC₅₀ | 300 |
| Compound P | IC₉₀ | 620 |
| Compound F | IC₅₀ | 260 |
| Compound F | IC₉₀ | 600 |
| Control antibody | IC₅₀ | 160 |
| 3(IL23A) | | |
| Control antibody | IC₉₀ | 310 |
| 3(IL23A) | | |

### Example 9. Human IL-23 Dependent Mouse Splenocyte Assay (MSA)

A mouse splenocyte based assay was used to assess the ability of anti-human IL23 test samples to inhibit the induction of mouse IL17 by human recombinant IL23 and recombinant cynomolgus IL23 in mouse splenocyte cultures.

### Materials and Methods

Briefly, mononuclear cells from mouse spleens (female C57BL/6 less than 13 weeks of age; JAX) were isolated washed, counted and resuspended to 4×10\hat{ }6 cells/ml in a standard T cell media (TCM). One hundred microliters of the mIL2/splenocyte suspension was added to 96 well microtiter plates. Recombinant human IL23 (Boehringer Ingelheim Pharmaceuticals, Inc.) or recombinant cynomolgus IL23 (Boehringer Ingelheim Pharmaceuticals, Inc.) was diluted in TCM and pre-incubated for 2 hours at 37 C with TCM alone or with titrations of test samples. After the pre-incubation of test sample+IL23, 100 ul of the mixture was added to the cells and the test plates were incubated at 37 C with 5% CO₂-humidified air for 48 hr. Control samples received either TCM (unstimulated controls) or recombinant human IL23 diluted in TCM (stimulated controls) After the incubation, mouse IL17 levels were determined from the supernatant using the Quantikine® Mouse IL-17 Immunoassay according to the manufacturer's instructions (R&D Systems). Interpolated mIL17 pg/ml values were determined for each sample and converted to percent of control (POC). The POC was plotted versus concentration of the test sample and IC₅₀ and IC₉₀ values were calculated using a 4 Parameter Logistic Model enabled by the Excel add-in XLfit (Activity Base software, ID Business Solutions, Ltd.). The anti-IL23 test samples were analyzed with respect to the IC₅₀/IC₉₀ as described above and Geomeans were calculated across multiple experiments for each test sample and shown in Table 10.

### Results

The results in Table 10 show that the tested compounds were able to inhibit both human and cynomolgus-IL23 induced mouse splenocyte release of IL17.

TABLE 10

| | | huIL23 | CynoIL23 |
|---|---|---|---|
| | IC₅₀ | MSA pM | MSA pM |
| Compound ID | IC₉₀ | Geomean | Geomean |
| Compound M | IC₅₀ | 140 | 120 |
| Compound M | IC₉₀ | 1600 | 890 |
| Compound A | IC₅₀ | 180 | 120 |
| Compound A | IC₉₀ | 1700 | 730 |
| Compound N | IC₅₀ | 230 | 140 |
| Compound N | IC₉₀ | 2000 | 940 |
| Compound D | IC₅₀ | 190 | 160 |
| Compound D | IC₉₀ | 2200 | 1100 |
| Compound O | IC₅₀ | 210 | 120 |
| Compound O | IC₉₀ | 2200 | 800 |
| Compound E | IC₅₀ | 200 | 77 |
| Compound E | IC₉₀ | 1700 | 1200 |
| Compound P | IC₅₀ | 200 | 97 |
| Compound P | IC₉₀ | 1400 | 1500 |
| Compound F | IC₅₀ | 170 | 69 |
| Compound F | IC₉₀ | 2300 | 1500 |
| Control antibody | IC₅₀ | 53 | 17 |
| 3(IL23A) | | | |
| Control antibody | IC₉₀ | 350 | 240 |
| 3(IL23A) | | | |

### Example 10. Inhibition of IL23 Induced Phosphorylation of STAT3

IL23 engagement with its heterodimeric receptor complex (IL12Rβ1-IL23R) results in the downstream phosphorylation of Signal transducer and activator of transcription 3 (STAT3). Compounds were tested for the ability to inhibit 1123 induced STAT3 activation in DB stable transfected cells

### Materials and Methods

The cells were stimulated with a final concentration of 15 ng/ml of IL23 protein. This dose was estimated to be the EC60 according to previous experiments, while allowing for inhibition with the tested compound. Cells were plated, compound dosed, and IL-23 added (in that order) and incubated overnight. If the compound inhibited cell stimulation, STAT3 was downregulated, leading to less luciferase activity.

### Results

The results in Table 11 show that the tested compounds were able to inhibit IL23 induced phosphorylation of STAT3.

TABLE 11

| | | IL23A |
|---|---|---|
| | | pSTAT3 |
| | IC₅₀ | (MG) pM |
| Compound ID | IC₉₀ | Geomean |
| Compound M | IC₅₀ | 120 |
| Compound M | IC₉₀ | 300 |
| Compound A | IC₅₀ | 130 |
| Compound A | IC₉₀ | 1100 |
| Compound O | IC₅₀ | 160 |
| Compound O | IC₉₀ | 650 |
| Compound E | IC₅₀ | 140 |
| Compound E | IC₉₀ | 830 |
| Compound P | IC₅₀ | 69 |
| Compound P | IC₉₀ | 480 |
| Compound F | IC₅₀ | 90 |
| Compound F | IC₉₀ | 650 |
| Control antibody 3(IL23A) | IC₅₀ | 35 |
| Control antibody 3(IL23A) | IC₉₀ | 140 |

### Example 11. Further IL23-A Stat3 Assays

Further experiments were run similarly to Example 8 to test for inhibition of IL23 induced activation of STAT3.

### Methods and Materials

DB-STAT3Luc10 Clone 10 suspension cells were grown in RPMI1640+10% FBS. 20,000 cells were added per well of 96 well plates at 80 ul/well of cell suspension. 10 ul of one of the serially diluted test compounds was added to each well. 15 ng/mL of recombinant human IL-23 was added to each well, with certain wells contained only test compounds and no IL-23, for comparison. The plates were incubated overnight at 37° C./5% CO2. Luciferase activity was assayed using Steady-Glo (Promega and One-Glo (Promega and the results were read on Envision Reader.

### Results

The IC₅₀ and IC₉₀ for the tested compounds are shown in Table 12 and Table 13. These tables show that the compounds inhibited IL-23-dependent STAT3 activation in a dose dependent manner.

TABLE 12

| | IC₅₀ | IC₉₀ |
|---|---|---|
| Compound ID | (pM) | (pM) |
| Compound N | 235.2 | 873.7 |
| Control antibody 3(IL23A) | 96.5 | 192.6 |
| Compound D | 185.6 | 871.6 |
| Compound E | 211.9 | 965.1 |
| Control antibody 3(IL23A) | 104.3 | 198.8 |
| Compound G | 220.2 | 1151.0 |
| Compound C | 162.7 | 620.4 |
| Control antibody 3(IL23A) | 80.3 | 181.3 |

TABLE 13

| | First test | | Second test | | GEOMEAN | |
|---|---|---|---|---|---|---|
| | IC₅₀ | IC₉₀ | IC₅₀ | IC₉₀ | IC50 | IC90 |
| Compound ID | (pM) | (pM) | (pM) | (pM) | (pM) | (pM) |
| Control | | | 96.5 | 192.6 | 93.1 | 190.8 |
| antibody | | | | | | |
| 3(IL23A) | | | | | | |
| | | | 104.3 | 198.8 | | |
| | | | 80.3 | 181.3 | | |
| Compound N | 178.6 | 856.4 | 235.2 | 873.7 | 205.0 | 865.0 |
| Compound D | 178.1 | 657.2 | 185.6 | 871.6 | 181.8 | 756.8 |
| Compound E | 170.2 | 764.9 | 211.9 | 965.1 | 189.9 | 859.2 |
| Compound G | 187.1 | 600.5 | 220.2 | 1151.0 | 203.0 | 831.4 |
| Compound C | 134.9 | 352.6 | 162.7 | 620.4 | 148.1 | 467.7 |

### Example 12. Inhibition of Mouse IL17A and IL22 Release Induced by Human Recombinant IL23

Test compounds were assessed for their ability to inhibit human IL23 induced cytokine release in C57/Bl6 mice. IL17A and IL22 secretion are measured after intradermal injection of IL23

### Materials and Methods

Briefly, C57BL/6 female mice (7-10 weeks old, Charles River) were randomly divided into 8 groups, 8 animals/group and given a 100 μl intraperitoneal injection of either citrate buffer (20 mM NaCitrate, 115 mM NaCl, pH 6.0) or test compounds at equivalent molar dose of 1.3, 0.4 and 0.13 mg/kg vs. 1, 3 and 0.1 mg/kg respectively.

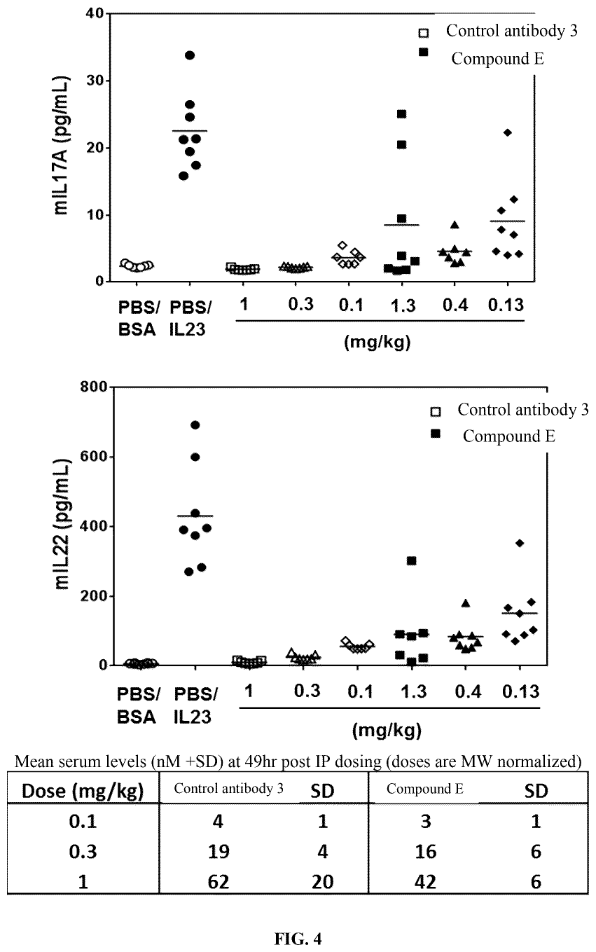

One hour after test compound dosing mice were anesthetized via isoflurane (Butler Schein) and given a 20 μl intradermal injection of either 0.1% BSA (Sigma) control or 15 μg/ml (0.3 μg) rhIL23 (generated in-house) diluted in saline (Invitrogen) to both ears. Intradermal challenges were repeated daily for 2 consecutive days. Twenty-four hours after the second challenge the mice were sacrificed via cervical dislocation and each ear was removed. Ear tissue was homogenized in 1 ml of homogenization buffer (HBSS (Gibco); 0.4% Triton X-100 (Sigma); 1× SigmaFast Protease Inhibitor (Sigma)) using a MP Biomedicals Fast-Prep 24 homogenizer. Homogenized samples are centrifuged at 4 C for 10 min and supernatant collected. Supernatants were assayed for the presence of mouse IL17A and IL22, using the Quantikine® Mouse IL-17 and mouse IL-22 Immunoassays according to the manufacturer's instructions (R&D Systems). Interpolated cytokine pg/ml values were determined for each sample. The mean pg/ml levels for each treatment group were determined and significance compared to control calculated using the One-way ANOVA followed by Dunnett's multiple comparisons test. Results are shown in FIG. 4.

### Results

The results in FIG. 4 show that treatment with a single intraperitoneal dose of test compound was able to significantly inhibit the release of mouse IL17 and IL22 in the skin induced by two daily consecutive intra dermal injections of recombinant human IL23.

### Example 13. Inhibition of Exogenous Human TNF-Alpha Dependent Cytokine Release in C57/Bl6 Mice

Test compounds were assessed for their ability to inhibit human TNF induced cytokine release in C57/Bl6 mice after exogenous exposure to human TNF. Serum KC and IL-6 secretion are measured following intraperitoneal administration of human TNF.

### Materials and Methods

Briefly, C57BL/6 female mice (8-9 weeks old, Jackson Labs) were randomly divided into 8 groups, 8 animals/group and given a 200 μl intraperitoneal injection of either phosphate buffered saline (Sigma) or test compound at equivalent molar dose of 13.3, 4 and 1.3 mg/kg vs. 10, 3 and 1 mg/kg respectively.

Two hour after test compound dosing mice were anesthetized via isoflurane (Butler Schein) and given a 200p0 intraperitoneal injection of either 0.1% BSA control or 15 μg/ml (3 μg) rhTNF (R&D Systems) diluted in saline (Sigma). Two hours after the TNF challenge the mice were anesthetized via isoflurane, whole blood was collected and mice were then sacrificed via cervical dislocation. Whole blood was centrifuged at 12,000 rpm for 10 minutes and plasma collected. Plasma was assayed for the presence of mouse KC and IL-6, using the MultiPlex® Mouse KC and mouse IL-6 Immunoassays according to the manufacturer's instructions (MSD). Interpolated cytokine pg/ml values were determined for each sample. The mean pg/ml levels for each treatment group were determined and significance compared to control calculated using the One-way ANOVA followed by Dunnett's multiple comparisons test. Results are shown in Figure X.

### Results

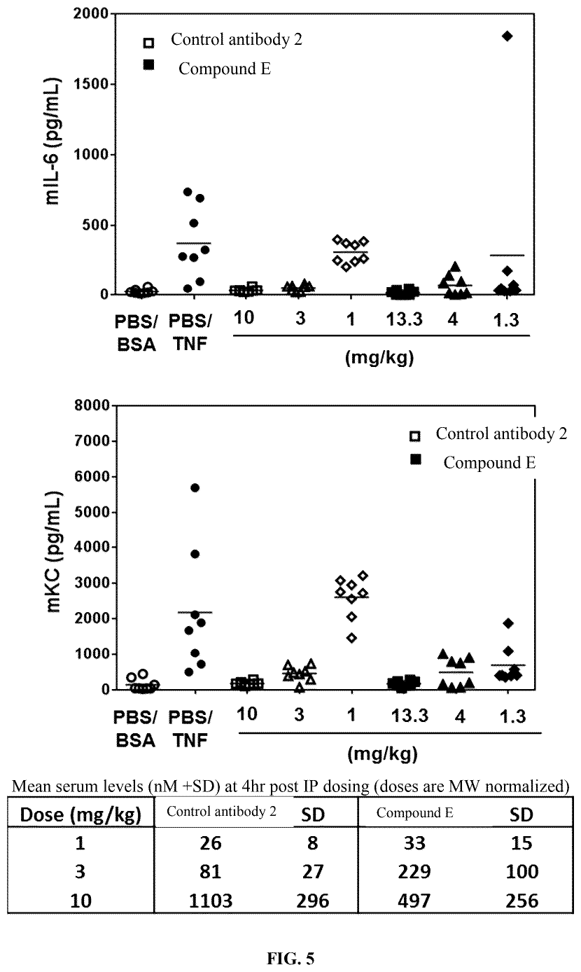

The results in FIG. 5 show that treatment with a single intraperitoneal dose of test compound was able to significantly inhibit the release of mouse KC and IL-6 in serum by intraperitoneal injection of recombinant human TNF.

### Example 14. Pharmacokinetics of Compounds in Cynomolgus Monkeys

### Materials and Methods

Single intravenous (IV) dose PK studies for two pairs of compounds (Compound M and Compound A; and Compound O and Compound E) were conducted in male cynomolgus monkeys (N=3 per group) naïve to biologics, and conducted according to the guidelines of Institutional Animal Care and Use Committee. IV doses were administered at 1 mg/kg as 10 min IV infusion. Serum samples were collected at pre-dose, 1, 4, 8 hr on the day of dosing, and 1, 2, 3, 4, 5, 7, 10, 14, 21, 28, 35, and 42 (1008 hr) days post dosing for Compound M and Compound A; and only up to Day 14 for Compound O and Compound E. Serum concentrations of the dosed molecules were measured by a ligand binding assay (ELISA).

Calibration standard curve and quality control (QC) samples were prepared in 100% serum for each analyte. Each standard curve consisted of seven non-zero points starting at 10240 ng/mL then serially diluted 3×. A blank sample (matrix without analyte) was also included. Four QC samples at low, medium, and high ranges were prepared starting at 2560 ng/mL then serially diluted four-fold. The standard curve and QC samples were stored frozen until sample analysis at which time they were diluted 20 times to mimic study samples. The standard curve and QC samples were included in duplicate during each analytical run. The lower and upper limits of quantification were defined as the lowest and highest standard curve points to reproducible have a back-calculated concentration that does not exceed 25 percent (%) of the nominal concentration. The acceptance criterion for the standard curve points and QC samples was 25 percent (%) of the nominal concentration.

Nunc ELISA plates were coated with 1 μL of monkey adsorbed goat anti-human IgG (Southern Biotech) as the capture reagent and incubated overnight at 2-8° C. After washing and blocking the plates with the wash buffer (0.05% (v/v) Tween 20 in phosphate buffered saline (PBS)) and blocking buffer (5% bovine serum albumin (BSA) in PBS), standard, QC, and unknown samples, diluted 1:20, 1:400 and 1:8000 with 5% monkey serum (monkey serum from Innovative Research) were added to the plate wells and incubated for 1 hour at room temperature. The plate wells were washed with the washing buffer and added with monkey adsorbed biotinylated goat anti-human IgG (Southern Biotech) as the secondary reagent and incubated at room temperature for 1 hour. The plates were washed 3 times and added with 100 μL of 1 μg/mL peroxidase-conjugated streptavidin for 15 min at room temperature, followed by further 3 times washing and the addition of 100 μL of 3,3′,5,5′-Tetramethylbenzidine (TMB, BioFX) substrate for 3-4 min at room temperature. The reaction was stopped by adding 100 μL of stop solution (BioFX) and the absorbance was measured using Molecular Devices plate reader with SoftmaxPro software, version 5.4.1.

### Results

Single IV dose PK studies for two pairs of test compounds (Compound M and Compound A; and Compound O and Compound E) were conducted in male cynomolgus monkeys (N=3 per group) naïve to biologics. The test compounds were dosed at 1 mg/kg as 10 min IV infusion. Serum samples were collected at pre-dose, 1, 4, 8 hr on the day of dosing, and 1, 2, 3, 4, 5, 7, 10, 14, 21, 28, 35, and 42 (1008 hr) days post dosing for Compound M and Compound A; and only up to Day 14 for Compound O and Compound E. Serum concentrations of the dosed molecules were measured by a ligand binding assay (ELISA).

Serum concentrations (mean and SD) for each of the molecules are summarized in Table 14.

TABLE 14
Serum concentrations (mean ± SD, N = 3) for the test compounds in cynomolgus monkey

| | Compound M | | Compound A | | Compound O | | Compound E | |
|---|---|---|---|---|---|---|---|---|
| Time | Mean | SD | Mean | SD | Mean | SD | Mean | SD |
| (day) | (nM) | (nM) | (nM) | (nM) | (nM) | (nM) | (nM) | (nM) |
| 0 | 0 | 0 | 0 | 0 | 0 | 0 | 0 | 0 |
| 0.042 | 89.62 | 26.41 | 115.60 | 15.44 | 96.69 | 23.15 | 107.57 | 10.43 |
| 0.167 | 75.30 | 17.97 | 108.03 | 22.92 | 92.29 | 19.75 | 102.36 | 4.81 |
| 0.333 | 69.71 | 9.19 | 89.15 | 16.32 | 65.27 | 11.53 | 86.45 | 10.65 |
| 1 | 54.49 | 13.94 | 63.68 | 6.69 | 30.10 | 12.41 | 52.87 | 8.85 |
| 2 | 35.17 | 8.93 | 49.95 | 6.04 | 7.44 | 3.22 | 32.51 | 6.30 |
| 3 | 18.58 | 6.66 | 43.51 | 6.64 | 4.14 | 0.70 | 24.65 | 6.49 |
| 4 | 13.86 | 5.61 | 40.02 | 8.50 | 2.76 | 0.25 | 19.02 | 4.84 |
| 5 | 9.55 | 3.40 | 43.69 | 19.63 | 1.96 | 0.09 | 13.98 | 3.84 |
| 7 | 3.76 | 1.31 | 27.84 | 3.29 | 1.08 | 0.21 | 8.49 | 3.38 |
| 10 | BQL | BQL | BQL | BQL | BQL | BQL | 0.3797 | NA |
| 14 | BQL | BQL | BQL | BQL | BQL | BQL | BQL | BQL |
| 21 | BQL | BQL | BQL | BQL | | | | |
| 28 | BQL | BQL | BQL | BQL | | | | |
| 35 | BQL | BQL | BQL | BQL | | | | |
| 42 | BQL | BQL | BQL | BQL | | | | |

BQL: below quantitation limit
NC: not calculated, N = 1

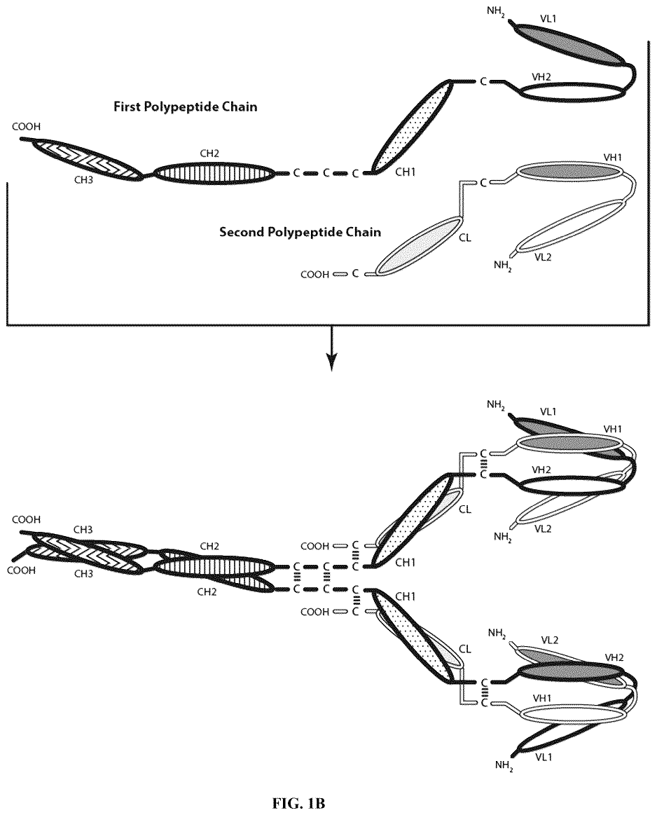

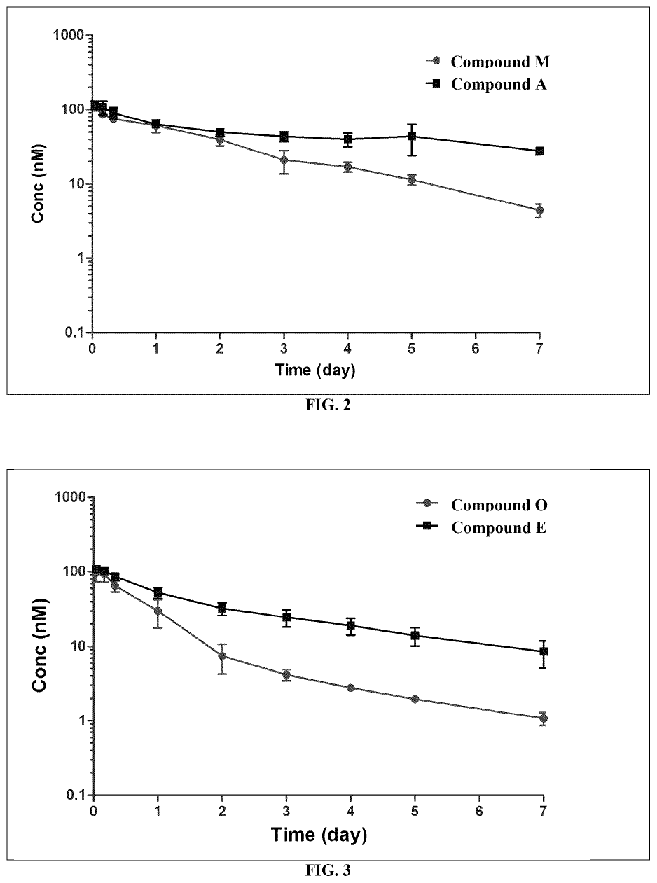

Pharmacokinetic (PK) parameters of these test compounds were calculated using the software Phoenix WinNonlin 6.1 (Certara, MD, USA) using non-compartmental approach for IV infusion dose. Serum samples that showed precipitous drop in the concentrations at any time point after dosing and all subsequent samples in that particular animal were excluded from the PK parameter estimation. Additional analysis showed that this sudden drop in the concentrations after the first few days was due to the development of anti-compound antibodies for a humanized biologic molecule in monkey. Only the first seven day data from individual animals were included in the PK analysis. Concentration-time plots are shown in FIGS. 2 and 3 for the two pairs of test compounds. Key PK parameters (mean±SD) for the two pairs of test compounds are summarized in Table 15.

TABLE 15
Key PK parameters (mean ± SD; N = 3) of the two test compound
pairs in cynomolgus monkey after 1 mg/kg IV 10 min infusion dose

| | | CL | | | |
|---|---|---|---|---|---|
| Compound | AUC | (mL/day/ | Vss | T_{1/2} | MRT |
| ID | (nM · day) | kg) | (mL/kg) | (day) | (day) |
| Compound M | 186 ± 48.3 | 28.2 ± 7.8 | 62.4 ± 13.7 | 1.6 ± 0.04 | 2.2 ± 0.1 |
| Compound A | 592 ± 68.3 | 8.5 ± 1.1 | 71.5 ± 8.9 | 6.2 ± 0.5 | 8.4 ± 1.0 |
| Compound O | 98.0 ± 14.7 | 51.8 ± 7.8 | 76.1 ± 30.3 | 2.3 ± 0.8 | 1.4 ± 0.4 |
| Compound E | 244 ± 54.6 | 21.2 ± 4.5 | 65.0 ± 3.9 | 2.5 ± 0.3 | 3.1 ± 0.5 |

Compound A, the test compound with YTE mutation showed a 3.3-fold reduction in clearance (CL) and a 3.9-fold increase in terminal half-life (T½) compared to the corresponding test compound not containing YTE mutation (Compound M). Compound E, the test compound with YTE mutation showed a 2.4-fold reduction in CL compared to the corresponding test compound not containing YTE mutation (Compound 0).

### Example 15. Predicting Human PK and Human Dose of Exemplary Compound

### Predicting Human PK:

Human PK prediction of Compound E was done by allometric scaling from the PK parameters obtained in cynomolgus monkey using a factor of 2-fold reduction in clearance in humans compared to monkey while maintaining same volume of distribution. Thus, predicted clearance in humans is 12.1 mL/day/kg with a terminal half-life of 7.4 days.

### Predicting Human Dose:

Human dose prediction was done based on extensive exposure-efficacy data available from clinical trials of golimumab in diverse patient populations. Golimumab (Simponi®) is approved to treat rheumatoid arthritis (RA), ankylosing spondylitis (AS), and psoriatic arthritis (PsA) patients with 50 mg monthly subcutaneous (SC) doses, and ulcerative colitis (UC) patients with 100 mg monthly SC doses. Simponi® achieves a Ctrough of approximately 3.2 nM in RA patients (50 mg monthly SC doses) and 9.7 nM (100 mg monthly SC doses) in UC patients (Simponi® BLA, 2009; Sandborn, 2013). These are used as benchmarks for therapeutic Ctroughs for AS and CD, respectively. Ctrough levels at the clinically approved doses of Stelara are about 6 nM. Based on the observation of a 3-fold higher potency of Compound E compared to ustekinumab (Stelara®) Ctrough values of ~2 nM are needed for Compound E to cover IL23. As the Ctrough concentration for covering TNF is greater than that for covering IL23, the 9.7 nM Ctrough for Simponi® was used for dose projections.

Compartmental modeling of PK data in the cynomolgus monkey (2-compartment model) followed by Monte-Carlo simulations using a 2-fold reduction scaling of CL, 73% bioavailability, and simultaneously varying clearance and distributional rate constants with a nominal 30% CV and log-normal distribution shows that 54 mg (90% confidence intervals 31-90 mg) SC doses administered every 2 weeks will maintain a Ctrough of 9.7 nM.

### Example 16. Purification of Compounds

### Methods

Compounds were purified using Mab Select SuRe as an affinity purification step. High salt washes are avoided in order to prevent aggregation. Elution was performed using Sodium Acetate buffer pH 3.5. Following Mab Select SuRE purification the sample was neutralized and applied to a Hydroxyapatite Type I resin and eluted using various concentrations of phosphate buffer. Monomer peak elutes ~140 mM NaPhosphate 100 mM NaCl pH 7.0 and aggregate peak eluted at ~200 mM NaPhosphate 100 mM NaCl pH 7.0. Following hydroxyapatite, the sample was consistently >95% monomer.

Sedimentation velocity (SV) experiment via Analytical ultracentrifugation (AUC) was used to provide information on sample purity and aggregation states. Samples were centrifuged in an optima XL-I (Beckman Coulter, Fullerton, CA) at 20° C. using an An60Ti four-hole rotor running at 40,000 rpm. The sedimentation process was monitored by ultraviolet absorbance at 280 nm, using corresponding dilution buffer as reference buffer. The variation in the concentration distribution in the ultracentrifuge cell with time was collected using XL-I operating software and was analyzed using the continuous c(S) distribution model in the SEDFIT software (version 14.1) to give the distribution of sedimentation coefficient. Monomer percentage was calculated based on the integrated peak area.

### Results

The results of purification of the compounds are shown in Table 16. The data show that the compounds have high purity and homogeneity indicating good stability.

TABLE 16

| Parameter | Compound A | Compound E |
|---|---|---|
| Percent monomer | 99.4 | 99.0 |
| (sedimentation velocity) | | |

### Example 17: Mass Spectrometry Profile of Compounds

### Methods

### Native Sample

This procedure yielded the intact mass of the compound or protein. 2 ul of sample was injected onto an Agilent PoroShell 300SB-C8 column, 5 um, (75×1.0 mm). The column temperature was 80° C. and flow rate was 50 ul/min. The compound or protein was eluted off the column with a gradient from 20% B at 0 minutes to 85% B at 10 minutes. Mobile phase A was Water/Acetonitrile/Formic Acid (99/1/0.1) and Mobile phase B was Acetonitrile/Water/Formic Acid (95/5/0.1). The effluent was directed to an Agilent 6210 TOF mass spectrometer, which was scanned from mass 600 to mass 3200. The raw data was deconvoluted with the program MassHunter.

### Reduced Sample

This procedure yielded the mass of the protein or the light chain and the mass of the heavy chain. 2 ul of 50 mM TCEP was added to 10 ul of sample and 10 ul of 8M Guanidine and incubated for 15 minutes at 37° C. 2 ul of this sample was injected as above, with the following differences: the column temperature was 60° C. and the mass range was 600-2000.

### Deglycosylated Sample

This procedure yielded the deglycosylated mass of the protein or the light chain and the heavy chain. 10 ul of sample, 10 ul of 200 mM NH₄HCO₃, 2 ul 50 mM TCEP, and 1 ul (1:10) PNGase F (or 1 uL QA deglycosylation mix if O-linked glycosylations were present) were incubated for 3 hours at 37° C. The incubation was increased to overnight for heavily glycosylated samples. Then, 25 ul 8M Guanidine and 4 ul of 50 mM TCEP were added and incubated for 15 minutes at 37° C. This sample was injected as above for reduced sample.

### Protein Peptide Mapping by Mass Spectrometry

25 ul of sample was added to 25 ul of 8M urea in 400 mM ammonium bicarbonate. Sul of 50 mM TCEP was then added and the sample was incubated for 15 minutes at 60° C. After cooling the sample to room temperature, Sul of 150 mM iodoacetamide was added and the sample was incubated at room temperature for 15 minutes. After adding 40 ul of water, Sul of trypsin in 1 mM HCl was added to give a final enzyme: substrate ratio of 1:50. The sample was incubated at 37° C. overnight. Sul was then injected onto a Thermo Hypurity C18 column, 100×1.0 mm. Flow rate was 80 ul/min. The protein was eluted off the column with a gradient from 0% B at 0 minutes to 40% B at 33 minutes. Mobile phase A was Water/Acetonitrile/Formic Acid (99/1/0.1) and Mobile phase B was Acetonitrile/Water/Formic Acid (95/5/0.1). The effluent was directed to a Thermo Orbitrap Velos mass spectrometer. The first scan event was in the FT, and scans from mass 300 to mass 2000 with a resolution of 30,000. The second through the seventh scan events were in the IT (ion trap) and fragmented the 6 most intense ions from the first scan event. Peptides containing glycosylation were profiled by manual extraction and percentages calculated based on peak heights.

### Results

The results are shown in Table 17. The data indicate the intended amino acid sequence and structure has been expressed and recovered without unexpected heterogeneity. The glycosylation pattern is typical of a conventional antibody expressed in CHO cells and does not show any atypical structures.

TABLE 17

| Parameter | Compound A | Compound E |
|---|---|---|
| Mass Spectrometry: Intact | Intact/Matches | Intact/Matches |
| Molecular Weight Profile | Sequence | Sequence |
| Mass Spectrometry: | Not | Similar |
| Glycosylation Profile | Determined | to CHO |
| | | expressed |
| | | IgG |

### Example 18: Thermal Stability of Compounds

### Methods

Thermal unfolding and aggregation of 2 mg/ml solutions of the compounds in phosphate buffer were monitored from 20° C. to 110° C. at a scan rate of 60° C./hr via an automated capillary DSC (MicroCal, LLC, Boston). Two scans with the corresponding buffer were performed to establish instrument thermal history and to obtain the instrument baseline for each sample, with the average of these scans subtracted from the subsequent protein thermogram to obtain the apparent heat capacity. Normalized scans were then analyzed with Origin 7.0. Pre-transition baselines were subtracted from each resulting heat capacity thermogram, to give the resulting excess heat capacity (Cp,ex) as a function of temperature. Reported values of transition temperatures (Tm) represent positions of peak maxima determined by visual inspection of the experimental thermograms.

### Results

The results are shown in Table 18. The data show that the compounds are stable and would predict the ability to have a long shelf-life.

TABLE 18

| Parameter | Compound A | Compound E |
|---|---|---|
| Thermal | 57.9, 72.1, 82.9 | 67.6, 83.1 |
| Stability (° C.) | | |

### Example 19: Solubility of Compounds

### Methods

The compound samples were concentrated gradually to a concentration as high as possible without precipitation observed using Amicon Ultra centrifugal filter with cut-off molecular weight of 50,000 Dalton (Millipore, Billerica). The concentrated protein solutions were then analyzed in SV experiment via AUC to provide information on sample purity and aggregation states (refer to Example 16 regarding purification for method details).

### Results

The results are shown in Table 19. The data show that the compounds are soluble and stable retaining a high percentage of monomer without formulation or added excipients.

TABLE 19

| Parameter | Compound A | Compound E |
|---|---|---|
| Solubility | 48 mg/ml | 51 mg/ml |
| (Concentration) | | |
| Percent Monomer | 98.6 | 97.0 |

### Example 20: Valence of Compounds

### Methods

The valence measurement for the compound samples in 50 mM KCl and 10 mM sodium acetate buffer at pH 5.0 was performed on a Beckman Coulter (Fullerton, California) ProteomeLab PA800™ apparatus equipped with an ultraviolet (UV) absorbance detector, with a working wavelength of 214 nm. The system was maintained at 20° C. and an eCap amine capillary with an inner diameter of 50 μm (Beckman Coulter, part #477431) was used. The capillary was rinsed with 100 mM NaOH, amine regeneration solution (Beckman Coulter, part #477433) and running buffer before each sample injection. Migration times for the samples were measured at voltages of 10 kV, 14 kV, and 18 kV. Dimethylformamide (DMF) (0.005%) (Pierce) was used as an electroosmotic flow (EOF) marker. Data were acquired using 32 Karat™ software (v7.0). Diffusion coefficient was determined from SV experiment via AUC.

### Results

The valence data (see Table 20) indicate colloidal stability of the compounds in solution, i.e. net interaction of protein and protein in solution. The compounds with valence greater than 15 have strong net repulsive interaction and high potential to be formulated at high concentration.

TABLE 20

| Parameter | Compound A | Compound E |
|---|---|---|
| Valence, pH 5.0 | 20.9 | 24.4 |

### Example 21: Predicted In Silico Immunogenicity

### Methods

Immunogenicity of protein therapeutics was predicted in silico by utilizing a computational tool, EpiMatrix that was developed by EpiVax, Inc. (Providence, RI). EpiMatrix incorporates the prediction of T-helper epitope as well as the T-regitope, of which the former is to provoke an immune response while the latter is inhibitory. Briefly, the protein sequence was first parsed into overlapping 9-mer peptide frames that has been proven the core of class II HLA binding. The binding potential of 9-mer peptides to each of eight common class II HLA alleles are evaluated based on experimental data or computational prediction. A score is generated to reflect the binding potential of the 9-mer peptide to each HLA allele and normalization is performed to make it possible to compare any 9-mer across multiple HLA alleles and enable immunogenicity prediction on a global scale. In the end the program generates an overall ‘immunogenicity score’, tReg Adjusted Epx Score, that together with other immunogenicity determinants helps to make an informed decision of the likelihood that the compounds will provoke an immune response in vivo.

### Results

The results are shown in Table 21. The overall immunogenicity scores for these compounds are low and predict that these compounds are not likely to illicit a strong immune response in vivo.

TABLE 21

| Parameter | Compound A | Compound E |
|---|---|---|
| EpiVax | −37.7, −35.6 | −31.1, −46.8 |

Normalization of allele-specific scores

### Example 22: Whole Blood Stability of Compounds

### Methods

A whole blood interference assay was developed on an Octet RED96 to detect the effects of non-specific binding or off-target binding for compounds in the presence of whole blood (WB). The compound solutions in whole blood and 1× kinetic running buffer (1×kb) were incubated at a temperature of 37° C. for 48 hours. Kinetic measurements for the incubated compound samples were performed with an Octet RED96 equipped with streptavidin (SA) biosensor tips (ForteBio, Menlo Park, CA) at 27° C. The ratio of the on-rates/binding signals in buffer and whole blood were reported. A ratio <2 was considered to show no interference.

### Results

The results are shown in Table 22.

TABLE 22

| Parameter | Compound A | Compound E |
|---|---|---|
| Ration of binding signal in whole | 1.5 | 1.4 |
| blood/kinctic buffer to hu TNFa | | |
| Ratio of binding signal in whole | 1.8 | 1.3 |
| blood/kinetic buffer | | |

### Example 23: Summary of Tested Parameters

A summary of the parameter data for certain compounds is shown in Table 23 below.

TABLE 23

| Parameter | Compound A | Compound E |
|---|---|---|
| Percent Monomer after two-step | 99.4% | 99% |
| purification process | | |
| Mass Spectrometry Profile | Intact | Intact |
| pI as determined by IEF | ~8.8 | ~8.8 |
| (heterogeneity) | | |
| Thermal Stability (differential | 57.9, 72.1, 82.9 | 67.6, 83.1 |
| scanning calorimetry) | | |
| Solubility | 48 mg/ml | 51 mg/ml |
| Valence at pH 5.0 | 20.9 | 24.4 |
| Predicted Immunogenicity | −37.69, −35.57 | −31.1, −46.8 |
| (EpiVax Score) | | |
| Whole Blood Stability (human WB, | Maintained | Maintained |
| 48 hrs at 37° C.); maintenance of | | |
| binding to IL23 and TNFa | | |

SEQUENCES

| SEQ ID | |
|---|---|
| NO | Sequence |
| 1 | EVQLVESGGGLVQPGRSLRLSCAASGFTFDDYAMHWVRQAPGKGLEWVSAITWNSGHI |
| | DYADSVEGRFTISRDNAKNSLYLQMNSLRAEDTAVYYCAKVSYLSTASSLDYWGQGTL |
| | VTVSS |
| 2 | DIQMTQSPSSLSASVGDRVTITCRASQGIRNYLAWYQQKPGKAPKLLIYAASTLQSGVPS |
| | RFSGSGSGTDFTLTISSLQPEDVATYYCQRYNRAPYTFGQGTKVEIK |
| 3 | QVQLVESGGGVVQPGRSLRLSCAASGFIFSSYAMHWVRQAPGNGLEWVAFMSYDGSNK |
| | KYADSVKGRFTISRDNSKNTLYLQMNSLRAEDTAVYYCARDRGIAAGGNYYYYGMDV |
| | WGQGTTVTVSS |
| 4 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIK |
| 5 | QVQLVESGGGVVQPGRSLRLSCAASGFIFSSYAMHWVRQAPGDGLEWVAFMSYDGSNK |
| | KYADSVKGRFTISRDNSKNTLYLQMNSLRAEDTAVYYCARDRGIAAGGNYYYYGMDV |
| | WGQGTTVTVSS |
| 6 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIK |
| 7 | QVQLVQSGAEVKKPGSSVKVSCKASGYTFTDQTIHWMRQAPGQGLEWIGYIYPRDDSPK |
| | YNENFKGKVTITADKSTSTAYMELSSLRSEDTAVYYCAIPDRSGYAWFIYWGQGTLVTV |
| | SS |
| 8 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIK |
| 9 | GGGSGGG |
| 10 | LGGGSG |
| 11 | FNRGES |
| 12 | VEPKSS |
| 13 | DIQMTQSPSSLSASVGDRVTITCRASQGIRNYLAWYQQKPGKAPKLLIYAASTLQSGVPS |
| | RFSGSGSGTDFTLTISSLQPEDVATYYCQRYNRAPYTFGQGTKVEIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGASTKGPSVFPLAPSSKSTSGGTAALGCLVKDYFPEPVTVSWNSGALTSGVHTFPAVL |
| | QSSGLYSLSSVVTVPSSSLGTQTYICNVNHKPSNTKVDKRVEPKSCDKTHTCPPCPAPEAA |
| | GGPSVFLFPPKPKDTLYITREPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSFFLYSKLTVD |
| | KSRWQQGNVFSCSVMHEALHNHYTQKSLSLSPGK |
| 14 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGEVQLVE |
| | SGGGLVQPGRSLRLSCAASGFTFDDYAMHWVRQAPGKGLEWVSAITWNSGHIDYADSV |
| | EGRFTISRDNAKNSLYLQMNSLRAEDTAVYYCAKVSYLSTASSLDYWGQGTLVTVSSLG |
| | GGSGRTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| 15 | DIQMTQSPSSLSASVGDRVTITCRASQGIRNYLAWYQQKPGKAPKLLIYAASTLQSGVPS |
| | RFSGSGSGTDFTLTISSLQPEDVATYYCQRYNRAPYTFGQGTKVEIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSFNRG |
| | ESASTKGPSVFPLAPSSKSTSGGTAALGCLVKDYFPEPVTVSWNSGALTSGVHTFPAVLQ |
| | SSGLYSLSSVVTVPSSSLGTQTYICNVNHKPSNTKVDKRVEPKSCDKTHTCPPCPAPEAAG |
| | GPSVFLFPPKPKDTLYITREPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLYSKLTVDKS |
| | RWQQGNVFSCSVMHEALHNHYTQKSLSLSPGK |
| 16 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGEVQLVE |
| | SGGGLVQPGRSLRLSCAASGFTFDDYAMHWVRQAPGKGLEWVSAITWNSGHIDYADSV |
| | EGRFTISRDNAKNSLYLQMNSLRAEDTAVYYCAKVSYLSTASSLDYWGQGTLVTVSSVE |
| | PKSSRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 17 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGEVQLVE |
| | SGGGLVQPGRSLRLSCAASGFTFDDYAMHWVRQAPGKGLEWVSAITWNSGHIDYADSV |
| | EGRFTISRDNAKNSLYLQMNSLRAEDTAVYYCAKVSYLSTASSLDYWGQGTLVTVSSLG |
| | GGSGASTKGPSVFPLAPSSKSTSGGTAALGCLVKDYFPEPVTVSWNSGALTSGVHTFPAV |
| | LQSSGLYSLSSVVTVPSSSLGTQTYICNVNHKPSNTKVDKRVEPKSCDKTHTCPPCPAPEA |
| | AGGPSVFLFPPKPKDTLYITREPEVTCVVVDVSHEDPEVKFNWYVDGVEVHNAKTKPRE |
| | EQYNSTYRVVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| 18 | DIQMTQSPSSLSASVGDRVTITCRASQGIRNYLAWYQQKPGKAPKLLIYAASTLQSGVPS |
| | RFSGSGSGTDFTLTISSLQPEDVATYYCQRYNRAPYTFGQGTKVEIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGRTVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC |
| 19 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGEVQLVE |
| | SGGGLVQPGRSLRLSCAASGFTFDDYAMHWVRQAPGKGLEWVSAITWNSGHIDYADSV |
| | EGRFTISRDNAKNSLYLQMNSLRAEDTAVYYCAKVSYLSTASSLDYWGQGTLVTVSSFN |
| | RGESASTKGPSVFPLAPSSKSTSGGTAALGCLVKDYFPEPVTVSWNSGALTSGVHTFPAV |
| | LQSSGLYSLSSVVTVPSSSLGTQTYICNVNHKPSNTKVDKRVEPKSCDKTHTCPPCPAPEA |
| | AGGPSVFLFPPKPKDTLYITREPEVTCVVVDVSHEDPEVKFNWYVDGVEVHNAKTKPRE |
| | EQYNSTYRVVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPGK |
| 20 | DIQMTQSPSSLSASVGDRVTITCRASQGIRNYLAWYQQKPGKAPKLLIYAASTLQSGVPS |
| | RFSGSGSGTDFTLTISSLQPEDVATYYCQRYNRAPYTFGQGTKVEIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSVEPK |
| | SSRTVAAPSVFIFPPSDEQLKSGTASVVCLLNNFYPREAKVQWKVDNALQSGNSQESVTE |
| | QDSKDSTYSLSSTLTLSKADYEKHKVYACEVTHQGLSSPVTKSFNRGEC |
| 21 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGNGLEWVAFMSYDGSNKKYADSV |
| | KGRFTISRDNSKNTLYLQMNSLRAEDTAVYYCARDRGIAAGGNYYYYGMDVWGQGTT |
| | VTVSSLGGGSGASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKRVEPKSCDKTHTC |
| | PPCPAPEAAGGPSVFLFPPKPKDTLYITREPEVTCVVVDVSHEDPEVKFNWYVDGVEVHN |
| | AKTKPREEQYNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPGK |
| 22 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGRTVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC |
| 23 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGNGLEWVAFMSYDGSNKKYADSV |
| | KGRFTISRDNSKNTLYLQMNSLRAEDTAVYYCARDRGIAAGGNYYYYGMDVWGQGTT |
| | VTVSSFNRGESASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKRVEPKSCDKTHTC |
| | PPCPAPEAAGGPSVFLFPPKPKDTLYITREPEVTCVVVDVSHEDPEVKFNWYVDGVEVHN |
| | AKTKPREEQYNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPGK |
| 24 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYTYPRDDSPKYNENF |
| | KGKVTITADKSTSTAYMELSSLRSEDTAVYYCAIPDRSGYAWFIYWGQGTLVTVSSVEPK |
| | SSRTVAAPSVFIFPPSDEQLKSGTASVVCLLNNFYPREAKVQWKVDNALQSGNSQESVTE |
| | QDSKDSTYSLSSTLTLSKADYEKHKVYACEVTHQGLSSPVTKSFNRGEC |
| 25 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGASTKGPSVFPLAPSSKSTSGGTAALGCLVKDYFPEPVTVSWNSGALTSGVHTFPAVL |
| | QSSGLYSLSSVVTVPSSSLGTQTYICNVNHKPSNTKVDKRVEPKSCDKTHTCPPCPAPEAA |
| | GGPSVFLFPPKPKDTLYITREPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSFFLYSKLTVD |
| | KSRWQQGNVFSCSVMHEALHNHYTQKSLSLSPGK |
| 26 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGNGLEWVAFMSYDGSNKKYADSV |
| | KGRFTISRDNSKNTLYLQMNSLRAEDTAVYYCARDRGIAAGGNYYYYGMDVWGQGTT |
| | VTVSSLGGGSGRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKSFNRGEC |
| 27 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSFNRG |
| | ESASTKGPSVFPLAPSSKSTSGGTAALGCLVKDYFPEPVTVSWNSGALTSGVHTFPAVLQ |
| | SSGLYSLSSVVTVPSSSLGTQTYICNVNHKPSNTKVDKRVEPKSCDKTHTCPPCPAPEAAG |
| | GPSVFLFPPKPKDTLYITREPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLYSKLTVDKS |
| | RWQQGNVFSCSVMHEALHNHYTQKSLSLSPGK |
| 28 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGNGLEWVAFMSYDGSNKKYADSV |
| | KGRFTISRDNSKNTLYLQMNSLRAEDTAVYYCARDRGIAAGGNYYYYGMDVWGQGTT |
| | VTVSSVEPKSSRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKSFNRGEC |
| 29 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGASTKGPSVFPLAPSSKSTSGGTAALGCLVKDYFPEPVTVSWNSGALTSGVHTFPAVL |
| | QSSGLYSLSSVVTVPSSSLGTQTYICNVNHKPSNTKVDKRVEPKSCDKTHTCPPCPAPEAA |
| | GGPSVFLFPPKPKDTLYITREPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSFFLYSKLTVD |
| | KSRWQQGNVFSCSVMHEALHNHYTQKSLSLSPGK |
| 30 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGDGLEWVAFMSYDGSNKKYADSV |
| | KGRFTISRDNSKNTLYLQMNSLRAEDTAVYYCARDRGIAAGGNYYYYGMDVWGQGTT |
| | VTVSSLGGGSGRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKSFNRGEC |
| 31 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGDGLEWVAFMSYDGSNKKYADSV |
| | KGRFTISRDNSKNTLYLQMNSLRAEDTAVYYCARDRGIAAGGNYYYYGMDVWGQGTT |
| | VTVSSLGGGSGASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKRVEPKSCDKTHTC |
| | PPCPAPEAAGGPSVFLFPPKPKDTLYITREPEVTCVVVDVSHEDPEVKFNWYVDGVEVHN |
| | AKTKPREEQYNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPGK |
| 32 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGRTVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC |
| 33 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSFNRG |
| | ESASTKGPSVFPLAPSSKSTSGGTAALGCLVKDYFPEPVTVSWNSGALTSGVHTFPAVLQ |
| | SSGLYSLSSVVTVPSSSLGTQTYICNVNHKPSNTKVDKRVEPKSCDKTHTCPPCPAPEAAG |
| | GPSVFLFPPKPKDTLYITREPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLYSKLTVDKS |
| | RWQQGNVFSCSVMHEALHNHYTQKSLSLSPGK |
| 34 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGDGLEWVAFMSYDGSNKKYADSV |
| | KGRFTISRDNSKNTLYLQMNSLRAEDTAVYYCARDRGIAAGGNYYYYGMDVWGQGTT |
| | VTVSSVEPKSSRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKSFNRGEC |
| 35 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGDGLEWVAFMSYDGSNKKYADSV |
| | KGRFTISRDNSKNTLYLQMNSLRAEDTAVYYCARDRGIAAGGNYYYYGMDVWGQGTT |
| | VTVSSFNRGESASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKRVEPKSCDKTHTC |
| | PPCPAPEAAGGPSVFLFPPKPKDTLYITREPEVTCVVVDVSHEDPEVKFNWYVDGVEVHN |
| | AKTKPREEQYNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPGK |
| 36 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSVEPK |
| | SSRTVAAPSVFIFPPSDEQLKSGTASVVCLLNNFYPREAKVQWKVDNALQSGNSQESVTE |
| | QDSKDSTYSLSSTLTLSKADYEKHKVYACEVTHQGLSSPVTKSFNRGEC |
| 37 | ASTKGPSVFPLAPCSRSTSESTAALGCLVKDYFPEPVTVSWNSGALTSGVHTFPAVLQSSG |
| | LYSLSSVVTVPSSSLGTKTYTCNVDHKPSNTKVDKRVESKYGPPCPPCPAPEFLGGPSVFL |
| | FPPKPKDTLMISRTPEVTCVVVDVSQEDPEVQFNWYVDGVEVHNAKTKPREEQFNSTYR |
| | VVSVLTVLHQDWLNGKEYKCKVSNKGLPSSIEKTISKAKGQPREPQVYTLPPSQEEMTK |
| | NQVSLTCLVKGFYPSDIAVEWESNGQPENNYKTTPPVLDSDGSFFLYSRLTVDKSRWQE |
| | GNVFSCSVMHEALHNHYTQKSLSLSLG |
| 38 | ASTKGPSVFPLAPCSRSTSESTAALGCLVKDYFPEPVTVSWNSGALTSGVHTFPAVLQSSG |
| | LYSLSSVVTVPSSSLGTKTYTCNVDHKPSNTKVDKRVESKYGPPCPPCPAPEFLGGPSVFL |
| | FPPKPKDTLYITREPEVTCVVVDVSQEDPEVQFNWYVDGVEVHNAKTKPREEQFNSTYR |
| | VVSVLTVLHQDWLNGKEYKCKVSNKGLPSSIEKTISKAKGQPREPQVYTLPPSQEEMTK |
| | NQVSLTCLVKGFYPSDIAVEWESNGQPENNYKTTPPVLDSDGSFFLYSRLTVDKSRWQE |
| | GNVFSCSVMHEALHNHYTQKSLSLSLG |
| 39 | ASTKGPSVFPLAPSSKSTSGGTAALGCLVKDYFPEPVTVSWNSGALTSGVHTFPAVLQSS |
| | GLYSLSSVVTVPSSSLGTQTYICNVNHKPSNTKVDKRVEPKSCDKTHTCPPCPAPELLGGP |
| | SVFLFPPKPKDTLMISRTPEVTCVVVDVSHEDPEVKFNWYVDGVEVHNAKTKPREEQYN |
| | STYRVVSVLTVLHQDWLNGKEYKCKVSNKALPAPIEKTISKAKGQPREPQVYTLPPSREE |
| | MTKNQVSLTCLVKGFYPSDIAVEWESNGQPENNYKTTPPVLDSDGSFFLYSKLTVDKSR |
| | WQQGNVFSCSVMHEALHNHYTQKSLSLSPG |
| 40 | EPKSCDKTHTCPPCP |
| 41 | RTVAAPSVFIFPPSDEQLKSGTASVVCLLNNFYPREAKVQWKVDNALQSGNSQESVTEQ |
| | DSKDSTYSLSSTLTLSKADYEKHKVYACEVTHQGLSSPVTKSFNRGEC |
| 42 | ASTKGPSVFPLAPSSKSTSGGTAALGCLVKDYFPEPVTVSWNSGALTSGVHTFPAVLQSS |
| | GLYSLSSVVTVPSSSLGTQTYICNVNHKPSNTKVDKRVEPKSCDKTHTCPPCPAPEAAGG |
| | PSVFLFPPKPKDTLYITREPEVTCVVVDVSHEDPEVKFNWYVDGVEVHNAKTKPREEQY |
| | NSTYRVVSVLTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLYSKLTVDKSR |
| | WQQGNVFSCSVMHEALHNHYTQKSLSLSPGK |
| 43 | ASTKGPSVFPLAPSSKSTSGGTAALGCLVKDYFPEPVTVSWNSGALTSGVHTFPAVLQSS |
| | GLYSLSSVVTVPSSSLGTQTYICNVNHKPSNTKVDKRVEPKSCDKTHTCPPCPAPEAAGG |
| | PSVFLFPPKPKDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREPQVYTLPPSRE |
| | EMTKNQVSLTCLVKGFYPSDIAVEWESNGQPENNYKTTPPVLDSDGSFFLYSKLTVDKSR |
| | WQQGNVFSCSVMHEALHNHYTQKSLSLSPG |
| 44 | DIQMTQSPSSLSASVGDRVTITCRASQGIRNYLAWYQQKPGKAPKLLIYAASTLQSGVPS |
| | RFSGSGSGTDFTLTISSLQPEDVATYYCQRYNRAPYTFGQGTKVEIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGASTKGPSVFPLAPSSKSTSGGTAALGCLVKDYFPEPVTVSWNSGALTSGVHTFPAVL |
| | QSSGLYSLSSVVTVPSSSLGTQTYICNVNHKPSNTKVDKRVEPKSCDKTHTCPPCPAPEAA |
| | GGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSFFLYSKLTVD |
| | KSRWQQGNVFSCSVMHEALHNHYTQKSLSLSPG |
| 45 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGEVQLVE |
| | SGGGLVQPGRSLRLSCAASGFTFDDYAMHWVRQAPGKGLEWVSAITWNSGHIDYADSV |
| | EGRFTISRDNAKNSLYLQMNSLRAEDTAVYYCAKVSYLSTASSLDYWGQGTLVTVSSLG |
| | GGSGRTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| 46 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGEVQLVE |
| | SGGGLVQPGRSLRLSCAASGFTFDDYAMHWVRQAPGKGLEWVSAITWNSGHIDYADSV |
| | EGRFTISRDNAKNSLYLQMNSLRAEDTAVYYCAKVSYLSTASSLDYWGQGTLVTVSSFN |
| | RGESASTKGPSVFPLAPSSKSTSGGTAALGCLVKDYFPEPVTVSWNSGALTSGVHTFPAV |
| | LQSSGLYSLSSVVTVPSSSLGTQTYICNVNHKPSNTKVDKRVEPKSCDKTHTCPPCPAPEA |
| | AGGPSVFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPG |
| 47 | DIQMTQSPSSLSASVGDRVTITCRASQGIRNYLAWYQQKPGKAPKLLIYAASTLQSGVPS |
| | RFSGSGSGTDFTLTISSLQPEDVATYYCQRYNRAPYTFGQGTKVEIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSVEPK |
| | SSRTVAAPSVFIFPPSDEQLKSGTASVVCLLNNFYPREAKVQWKVDNALQSGNSQESVTE |
| | QDSKDSTYSLSSTLTLSKADYEKHKVYACEVTHQGLSSPVTKSFNRGEC |
| 48 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGNGLEWVAFMSYDGSNKKYADSV |
| | KGRFTISRDNSKNTLYLQMNSLRAEDTAVYYCARDRGIAAGGNYYYYGMDVWGQGTT |
| | VTVSSLGGGSGASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKRVEPKSCDKTHTC |
| | PPCPAPEAAGGPSVFLFPPKPKDTLMISRTPEVTCVVVDVSHEDPEVKFNWYVDGVEVHN |
| | AKTKPREEQYNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPG |
| 49 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGRTVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC |
| 50 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGNGLEWVAFMSYDGSNKKYADSV |
| | KGRFTISRDNSKNTLYLQMNSLRAEDTAVYYCARDRGIAAGGNYYYYGMDVWGQGTT |
| | VTVSSFNRGESASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKRVEPKSCDKTHTC |
| | PPCPAPEAAGGPSVFLFPPKPKDTLMISRTPEVTCVVVDVSHEDPEVKFNWYVDGVEVHN |
| | AKTKPREEQYNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPG |
| 51 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSVEPK |
| | SSRTVAAPSVFIFPPSDEQLKSGTASVVCLLNNFYPREAKVQWKVDNALQSGNSQESVTE |
| | QDSKDSTYSLSSTLTLSKADYEKHKVYACEVTHQGLSSPVTKSFNRGEC |
| 52 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGASTKGPSVFPLAPCSRSTSESTAALGCLVKDYFPEPVTVSWNSGALTSGVHTFPAVL |
| | QSSGLYSLSSVVTVPSSSLGTKTYTCNVDHKPSNTKVDKRVESKYGPPCPPCPAPEFLGGP |
| | SVFLFPPKPKDTLMISRTPEVTCVVVDVSQEDPEVQFNWYVDGVEVHNAKTKPREEQFN |
| | STYRVVSVLTVLHQDWLNGKEYKCKVSNKGLPSSIEKTISKAKGQPREPQVYTLPPSQEE |
| | MTKNQVSLTCLVKGFYPSDIAVEWESNGQPENNYKTTPPVLDSDGSFFLYSRLTVDKSR |
| | WQEGNVFSCSVMHEALHNHYTQKSLSLSLG |
| 53 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGNGLEWVAFMSYDGSNKKYADSV |
| | KGRFTISRDNSKNTLYLQMNSLRAEDTAVYYCARDRGIAAGGNYYYYGMDVWGQGTT |
| | VTVSSLGGGSGRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKSFNRGEC |
| 54 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGASTKGPSVFPLAPCSRSTSESTAALGCLVKDYFPEPVTVSWNSGALTSGVHTFPAVL |
| | QSSGLYSLSSVVTVPSSSLGTKTYTCNVDHKPSNTKVDKRVESKYGPPCPPCPAPEFLGGP |
| | SVFLFPPKPKDTLYITREPEVTCVVVDVSQEDPEVQFNWYVDGVEVHNAKTKPREEQFNS |
| | TYRVVSVLTVLHQDWLNGKEYKCKVSNKGLPSSIEKTISKAKGQPREPQVYTLPPSQEEM |
| | TKNQVSLTCLVKGFYPSDIAVEWESNGQPENNYKTTPPVLDSDGSFFLYSRLTVDKSRW |
| | QEGNVFSCSVMHEALHNHYTQKSLSLSLG |
| 55 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGNGLEWVAFMSYDGSNKKYADSV |
| | KGRFTISRDNSKNTLYLQMNSLRAEDTAVYYCARDRGIAAGGNYYYYGMDVWGQGTT |
| | VTVSSLGGGSGRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKSFNRGEC |
| 56 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGASTKGPSVFPLAPCSRSTSESTAALGCLVKDYFPEPVTVSWNSGALTSGVHTFPAVL |
| | QSSGLYSLSSVVTVPSSSLGTKTYTCNVDHKPSNTKVDKRVESKYGPPCPPCPAPEFLGGP |
| | SVFLFPPKPKDTLMISRTPEVTCVVVDVSQEDPEVQFNWYVDGVEVHNAKTKPREEQFN |
| | STYRVVSVLTVLHQDWLNGKEYKCKVSNKGLPSSIEKTISKAKGQPREPQVYTLPPSQEE |
| | MTKNQVSLTCLVKGFYPSDIAVEWESNGQPENNYKTTPPVLDSDGSFFLYSRLTVDKSR |
| | WQEGNVFSCSVMHEALHNHYTQKSLSLSLG |
| 57 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGNGLEWVAFMSYDGSNKKYADSV |
| | KGRFTISRDNSKNTLYLQMNSLRAEDTAVYYCARDRGIAAGGNYYYYGMDVWGQGTT |
| | VTVSSLGGGSGRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKSFNRGEC |
| 58 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGASTKGPSVFPLAPCSRSTSESTAALGCLVKDYFPEPVTVSWNSGALTSGVHTFPAVL |
| | QSSGLYSLSSVVTVPSSSLGTKTYTCNVDHKPSNTKVDKRVESKYGPPCPPCPAPEFLGGP |
| | SVFLFPPKPKDTLYITREPEVTCVVVDVSQEDPEVQFNWYVDGVEVHNAKTKPREEQFNS |
| | TYRVVSVLTVLHQDWLNGKEYKCKVSNKGLPSSIEKTISKAKGQPREPQVYTLPPSQEEM |
| | TKNQVSLTCLVKGFYPSDIAVEWESNGQPENNYKTTPPVLDSDGSFFLYSRLTVDKSRW |
| | QEGNVFSCSVMHEALHNHYTQKSLSLSLG |
| 59 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGNGLEWVAFMSYDGSNKKYADSV |
| | KGRFTISRDNSKNTLYLQMNSLRAEDTAVYYCARDRGIAAGGNYYYYGMDVWGQGTT |
| | VTVSSLGGGSGRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKSFNRGEC |
| 60 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSFNRG |
| | ESASTKGPSVFPLAPSSKSTSGGTAALGCLVKDYFPEPVTVSWNSGALTSGVHTFPAVLQ |
| | SSGLYSLSSVVTVPSSSLGTQTYICNVNHKPSNTKVDKRVEPKSCDKTHTCPPCPAPEAAG |
| | GPSVFLFPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLYSKLTVDKS |
| | RWQQGNVFSCSVMHEALHNHYTQKSLSLSPG |
| 61 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGNGLEWVAFMSYDGSNKKYADSV |
| | KGRFTISRDNSKNTLYLQMNSLRAEDTAVYYCARDRGIAAGGNYYYYGMDVWGQGTT |
| | VTVSSVEPKSSRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKSFNRGEC |
| 62 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSFNRG |
| | ESASTKGPSVFPLAPSSKSTSGGTAALGCLVKDYFPEPVTVSWNSGALTSGVHTFPAVLQ |
| | SSGLYSLSSVVTVPSSSLGTQTYICNVNHKPSNTKVDKRVEPKSCDKTHTCPPCPAPELLG |
| | GPSVFLFPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLYSKLTVDKS |
| | RWQQGNVFSCSVMHEALHNHYTQKSLSLSPG |
| 63 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGNGLEWVAFMSYDGSNKKYADSV |
| | KGRFTISRDNSKNTLYLQMNSLRAEDTAVYYCARDRGIAAGGNYYYYGMDVWGQGTT |
| | VTVSSVEPKSSRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKSFNRGEC |
| 64 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSFNRG |
| | ESASTKGPSVFPLAPCSRSTSESTAALGCLVKDYFPEPVTVSWNSGALTSGVHTFPAVLQS |
| | SGLYSLSSVVTVPSSSLGTKTYTCNVDHKPSNTKVDKRVESKYGPPCPPCPAPEFLGGPSV |
| | FLFPPKPKDTLMISRTPEVTCVVVDVSQEDPEVQFNWYVDGVEVHNAKTKPREEQFNST |
| | YRVVSVLTVLHQDWLNGKEYKCKVSNKGLPSSIEKTISKAKGQPREPQVYTLPPSQEEMT |
| | KNQVSLTCLVKGFYPSDIAVEWESNGQPENNYKTTPPVLDSDGSFFLYSRLTVDKSRWQ |
| | EGNVFSCSVMHEALHNHYTQKSLSLSLG |
| 65 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGNGLEWVAFMSYDGSNKKYADSV |
| | KGRFTISRDNSKNTLYLQMNSLRAEDTAVYYCARDRGIAAGGNYYYYGMDVWGQGTT |
| | VTVSSVEPKSSRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKSFNRGEC |
| 66 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSFNRG |
| | ESASTKGPSVFPLAPCSRSTSESTAALGCLVKDYFPEPVTVSWNSGALTSGVHTFPAVLQS |
| | SGLYSLSSVVTVPSSSLGTKTYTCNVDHKPSNTKVDKRVESKYGPPCPPCPAPEFLGGPSV |
| | FLFPPKPKDTLYITREPEVTCVVVDVSQEDPEVQFNWYVDGVEVHNAKTKPREEQFNST |
| | YRVVSVLTVLHQDWLNGKEYKCKVSNKGLPSSIEKTISKAKGQPREPQVYTLPPSQEEMT |
| | KNQVSLTCLVKGFYPSDIAVEWESNGQPENNYKTTPPVLDSDGSFFLYSRLTVDKSRWQ |
| | EGNVFSCSVMHEALHNHYTQKSLSLSLG |
| 67 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGNGLEWVAFMSYDGSNKKYADSV |
| | KGRFTISRDNSKNTLYLQMNSLRAEDTAVYYCARDRGIAAGGNYYYYGMDVWGQGTT |
| | VTVSSVEPKSSRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKSFNRGEC |
| 68 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGNGLEWVAFMSYDGSNKKYADSV |
| | KGRFTISRDNSKNTLYLQMNSLRAEDTAVYYCARDRGIAAGGNYYYYGMDVWGQGTT |
| | VTVSSLGGGSGASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKRVEPKSCDKTHTC |
| | PPCPAPELLGGPSVFLFPPKPKDTLMISRTPEVTCVVVDVSHEDPEVKFNWYVDGVEVHN |
| | AKTKPREEQYNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPG |
| 69 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGRTVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC |
| 70 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGNGLEWVAFMSYDGSNKKYADSV |
| | KGRFTISRDNSKNTLYLQMNSLRAEDTAVYYCARDRGIAAGGNYYYYGMDVWGQGTT |
| | VTVSSLGGGSGASTKGPSVFPLAPCSRSTSESTAALGCLVKDYFPEPVTVSWNSGALTSG |
| | VHTFPAVLQSSGLYSLSSVVTVPSSSLGTKTYTCNVDHKPSNTKVDKRVESKYGPPCPPC |
| | PAPEFLGGPSVFLFPPKPKDTLMISRTPEVTCVVVDVSQEDPEVQFNWYVDGVEVHNAKT |
| | KPREEQFNSTYRVVSVLTVLHQDWLNGKEYKCKVSNKGLPSSIEKTISKAKGQPREPQVY |
| | TLPPSQEEMTKNQVSLTCLVKGFYPSDIAVEWESNGQPENNYKTTPPVLDSDGSFFLYSR |
| | LTVDKSRWQEGNVFSCSVMHEALHNHYTQKSLSLSLG |
| 71 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGRTVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC |
| 72 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGNGLEWVAFMSYDGSNKKYADSV |
| | KGRFTISRDNSKNTLYLQMNSLRAEDTAVYYCARDRGIAAGGNYYYYGMDVWGQGTT |
| | VTVSSLGGGSGASTKGPSVFPLAPCSRSTSESTAALGCLVKDYFPEPVTVSWNSGALTSG |
| | VHTFPAVLQSSGLYSLSSVVTVPSSSLGTKTYTCNVDHKPSNTKVDKRVESKYGPPCPPC |
| | PAPEFLGGPSVFLFPPKPKDTLYITREPEVTCVVVDVSQEDPEVQFNWYVDGVEVHNAKT |
| | KPREEQFNSTYRVVSVLTVLHQDWLNGKEYKCKVSNKGLPSSIEKTISKAKGQPREPQVY |
| | TLPPSQEEMTKNQVSLTCLVKGFYPSDIAVEWESNGQPENNYKTTPPVLDSDGSFFLYSR |
| | LTVDKSRWQEGNVFSCSVMHEALHNHYTQKSLSLSLG |
| 73 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGRTVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC |
| 74 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGNGLEWVAFMSYDGSNKKYADSV |
| | KGRFTISRDNSKNTLYLQMNSLRAEDTAVYYCARDRGIAAGGNYYYYGMDVWGQGTT |
| | VTVSSFNRGESASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKRVEPKSCDKTHTC |
| | PPCPAPELLGGPSVFLFPPKPKDTLMISRTPEVTCVVVDVSHEDPEVKFNWYVDGVEVHN |
| | AKTKPREEQYNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPG |
| 75 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSVEPK |
| | SSRTVAAPSVFIFPPSDEQLKSGTASVVCLLNNFYPREAKVQWKVDNALQSGNSQESVTE |
| | QDSKDSTYSLSSTLTLSKADYEKHKVYACEVTHQGLSSPVTKSFNRGEC |
| 76 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGNGLEWVAFMSYDGSNKKYADSV |
| | KGRFTISRDNSKNTLYLQMNSLRAEDTAVYYCARDRGIAAGGNYYYYGMDVWGQGTT |
| | VTVSSFNRGESASTKGPSVFPLAPCSRSTSESTAALGCLVKDYFPEPVTVSWNSGALTSGV |
| | HTFPAVLQSSGLYSLSSVVTVPSSSLGTKTYTCNVDHKPSNTKVDKRVESKYGPPCPPCP |
| | APEFLGGPSVFLFPPKPKDTLMISRTPEVTCVVVDVSQEDPEVQFNWYVDGVEVHNAKT |
| | KPREEQFNSTYRVVSVLTVLHQDWLNGKEYKCKVSNKGLPSSIEKTISKAKGQPREPQVY |
| | TLPPSQEEMTKNQVSLTCLVKGFYPSDIAVEWESNGQPENNYKTTPPVLDSDGSFFLYSR |
| | LTVDKSRWQEGNVFSCSVMHEALHNHYTQKSLSLSLG |
| 77 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSVEPK |
| | SSRTVAAPSVFIFPPSDEQLKSGTASVVCLLNNFYPREAKVQWKVDNALQSGNSQESVTE |
| | QDSKDSTYSLSSTLTLSKADYEKHKVYACEVTHQGLSSPVTKSFNRGEC |
| 78 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGNGLEWVAFMSYDGSNKKYADSV |
| | KGRFTISRDNSKNTLYLQMNSLRAEDTAVYYCARDRGIAAGGNYYYYGMDVWGQGTT |
| | VTVSSFNRGESASTKGPSVFPLAPCSRSTSESTAALGCLVKDYFPEPVTVSWNSGALTSGV |
| | HTFPAVLQSSGLYSLSSVVTVPSSSLGTKTYTCNVDHKPSNTKVDKRVESKYGPPCPPCP |
| | APEFLGGPSVFLFPPKPKDTLYITREPEVTCVVVDVSQEDPEVQFNWYVDGVEVHNAKT |
| | KPREEQFNSTYRVVSVLTVLHQDWLNGKEYKCKVSNKGLPSSIEKTISKAKGQPREPQVY |
| | TLPPSQEEMTKNQVSLTCLVKGFYPSDIAVEWESNGQPENNYKTTPPVLDSDGSFFLYSR |
| | LTVDKSRWQEGNVFSCSVMHEALHNHYTQKSLSLSLG |
| 79 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSVEPK |
| | SSRTVAAPSVFIFPPSDEQLKSGTASVVCLLNNFYPREAKVQWKVDNALQSGNSQESVTE |
| | QDSKDSTYSLSSTLTLSKADYEKHKVYACEVTHQGLSSPVTKSFNRGEC |
| 80 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGASTKGPSVFPLAPSSKSTSGGTAALGCLVKDYFPEPVTVSWNSGALTSGVHTFPAVL |
| | QSSGLYSLSSVVTVPSSSLGTQTYICNVNHKPSNTKVDKRVEPKSCDKTHTCPPCPAPEAA |
| | GGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSFFLYSKLTVD |
| | KSRWQQGNVFSCSVMHEALHNHYTQKSLSLSPG |
| 81 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGDGLEWVAFMSYDGSNKKYADSV |
| | KGRFTISRDNSKNTLYLQMNSLRAEDTAVYYCARDRGIAAGGNYYYYGMDVWGQGTT |
| | VTVSSLGGGSGRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKSFNRGEC |
| 82 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGASTKGPSVFPLAPSSKSTSGGTAALGCLVKDYFPEPVTVSWNSGALTSGVHTFPAVL |
| | QSSGLYSLSSVVTVPSSSLGTQTYICNVNHKPSNTKVDKRVEPKSCDKTHTCPPCPAPELL |
| | GGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSFFLYSKLTVD |
| | KSRWQQGNVFSCSVMHEALHNHYTQKSLSLSPG |
| 83 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGDGLEWVAFMSYDGSNKKYADSV |
| | KGRFTISRDNSKNTLYLQMNSLRAEDTAVYYCARDRGIAAGGNYYYYGMDVWGQGTT |
| | VTVSSLGGGSGRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKSFNRGEC |
| 84 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGASTKGPSVFPLAPCSRSTSESTAALGCLVKDYFPEPVTVSWNSGALTSGVHTFPAVL |
| | QSSGLYSLSSVVTVPSSSLGTKTYTCNVDHKPSNTKVDKRVESKYGPPCPPCPAPEFLGGP |
| | SVFLFPPKPKDTLMISRTPEVTCVVVDVSQEDPEVQFNWYVDGVEVHNAKTKPREEQFN |
| | STYRVVSVLTVLHQDWLNGKEYKCKVSNKGLPSSIEKTISKAKGQPREPQVYTLPPSQEE |
| | MTKNQVSLTCLVKGFYPSDIAVEWESNGQPENNYKTTPPVLDSDGSFFLYSRLTVDKSR |
| | WQEGNVFSCSVMHEALHNHYTQKSLSLSLG |
| 85 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGDGLEWVAFMSYDGSNKKYADSV |
| | KGRFTISRDNSKNTLYLQMNSLRAEDTAVYYCARDRGIAAGGNYYYYGMDVWGQGTT |
| | VTVSSLGGGSGRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKSFNRGEC |
| 86 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGASTKGPSVFPLAPCSRSTSESTAALGCLVKDYFPEPVTVSWNSGALTSGVHTFPAVL |
| | QSSGLYSLSSVVTVPSSSLGTKTYTCNVDHKPSNTKVDKRVESKYGPPCPPCPAPEFLGGP |
| | SVFLFPPKPKDTLYITREPEVTCVVVDVSQEDPEVQFNWYVDGVEVHNAKTKPREEQFNS |
| | TYRVVSVLTVLHQDWLNGKEYKCKVSNKGLPSSIEKTISKAKGQPREPQVYTLPPSQEEM |
| | TKNQVSLTCLVKGFYPSDIAVEWESNGQPENNYKTTPPVLDSDGSFFLYSRLTVDKSRW |
| | QEGNVFSCSVMHEALHNHYTQKSLSLSLG |
| 87 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGDGLEWVAFMSYDGSNKKYADSV |
| | KGRFTISRDNSKNTLYLQMNSLRAEDTAVYYCARDRGIAAGGNYYYYGMDVWGQGTT |
| | VTVSSLGGGSGRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKSFNRGEC |
| 88 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSFNRG |
| | ESASTKGPSVFPLAPSSKSTSGGTAALGCLVKDYFPEPVTVSWNSGALTSGVHTFPAVLQ |
| | SSGLYSLSSVVTVPSSSLGTQTYICNVNHKPSNTKVDKRVEPKSCDKTHTCPPCPAPEAAG |
| | GPSVFLFPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLYSKLTVDKS |
| | RWQQGNVFSCSVMHEALHNHYTQKSLSLSPG |
| 89 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGDGLEWVAFMSYDGSNKKYADSV |
| | KGRFTISRDNSKNTLYLQMNSLRAEDTAVYYCARDRGIAAGGNYYYYGMDVWGQGTT |
| | VTVSSVEPKSSRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKSFNRGEC |
| 90 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSFNRG |
| | ESASTKGPSVFPLAPSSKSTSGGTAALGCLVKDYFPEPVTVSWNSGALTSGVHTFPAVLQ |
| | SSGLYSLSSVVTVPSSSLGTQTYICNVNHKPSNTKVDKRVEPKSCDKTHTCPPCPAPELLG |
| | GPSVFLFPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLYSKLTVDKS |
| | RWQQGNVFSCSVMHEALHNHYTQKSLSLSPG |
| 91 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGDGLEWVAFMSYDGSNKKYADSV |
| | KGRFTISRDNSKNTLYLQMNSLRAEDTAVYYCARDRGIAAGGNYYYYGMDVWGQGTT |
| | VTVSSVEPKSSRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKSFNRGEC |
| 92 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSFNRG |
| | ESASTKGPSVFPLAPCSRSTSESTAALGCLVKDYFPEPVTVSWNSGALTSGVHTFPAVLQS |
| | SGLYSLSSVVTVPSSSLGTKTYTCNVDHKPSNTKVDKRVESKYGPPCPPCPAPEFLGGPSV |
| | FLFPPKPKDTLMISRTPEVTCVVVDVSQEDPEVQFNWYVDGVEVHNAKTKPREEQFNST |
| | YRVVSVLTVLHQDWLNGKEYKCKVSNKGLPSSIEKTISKAKGQPREPQVYTLPPSQEEMT |
| | KNQVSLTCLVKGFYPSDIAVEWESNGQPENNYKTTPPVLDSDGSFFLYSRLTVDKSRWQ |
| | EGNVFSCSVMHEALHNHYTQKSLSLSLG |
| 93 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGDGLEWVAFMSYDGSNKKYADSV |
| | KGRFTISRDNSKNTLYLQMNSLRAEDTAVYYCARDRGIAAGGNYYYYGMDVWGQGTT |
| | VTVSSVEPKSSRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKSFNRGEC |
| 94 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSFNRG |
| | ESASTKGPSVFPLAPCSRSTSESTAALGCLVKDYFPEPVTVSWNSGALTSGVHTFPAVLQS |
| | SGLYSLSSVVTVPSSSLGTKTYTCNVDHKPSNTKVDKRVESKYGPPCPPCPAPEFLGGPSV |
| | FLFPPKPKDTLYITREPEVTCVVVDVSQEDPEVQFNWYVDGVEVHNAKTKPREEQFNST |
| | YRVVSVLTVLHQDWLNGKEYKCKVSNKGLPSSIEKTISKAKGQPREPQVYTLPPSQEEMT |
| | KNQVSLTCLVKGFYPSDIAVEWESNGQPENNYKTTPPVLDSDGSFFLYSRLTVDKSRWQ |
| | EGNVFSCSVMHEALHNHYTQKSLSLSLG |
| 95 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGDGLEWVAFMSYDGSNKKYADSV |
| | KGRFTISRDNSKNTLYLQMNSLRAEDTAVYYCARDRGIAAGGNYYYYGMDVWGQGTT |
| | VTVSSVEPKSSRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKSFNRGEC |
| 96 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGDGLEWVAFMSYDGSNKKYADSV |
| | KGRFTISRDNSKNTLYLQMNSLRAEDTAVYYCARDRGIAAGGNYYYYGMDVWGQGTT |
| | VTVSSLGGGSGASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKRVEPKSCDKTHTC |
| | PPCPAPEAAGGPSVFLFPPKPKDTLMISRTPEVTCVVVDVSHEDPEVKFNWYVDGVEVHN |
| | AKTKPREEQYNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPG |
| 97 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGRTVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC |
| 98 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGDGLEWVAFMSYDGSNKKYADSV |
| | KGRFTISRDNSKNTLYLQMNSLRAEDTAVYYCARDRGIAAGGNYYYYGMDVWGQGTT |
| | VTVSSLGGGSGASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKRVEPKSCDKTHTC |
| | PPCPAPELLGGPSVFLFPPKPKDTLMISRTPEVTCVVVDVSHEDPEVKFNWYVDGVEVHN |
| | AKTKPREEQYNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPG |
| 99 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGRTVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC |
| 100 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGDGLEWVAFMSYDGSNKKYADSV |
| | KGRFTISRDNSKNTLYLQMNSLRAEDTAVYYCARDRGIAAGGNYYYYGMDVWGQGTT |
| | VTVSSLGGGSGASTKGPSVFPLAPCSRSTSESTAALGCLVKDYFPEPVTVSWNSGALTSG |
| | VHTFPAVLQSSGLYSLSSVVTVPSSSLGTKTYTCNVDHKPSNTKVDKRVESKYGPPCPPC |
| | PAPEFLGGPSVFLFPPKPKDTLMISRTPEVTCVVVDVSQEDPEVQFNWYVDGVEVHNAKT |
| | KPREFQFNSTYRVVSVLTVLHQDWLNGKEYKCKVSNKGLPSSIEKTISKAKGQPREPQVY |
| | TLPPSQEEMTKNQVSLTCLVKGFYPSDIAVEWESNGQPENNYKTTPPVLDSDGSFFLYSR |
| | LTVDKSRWQEGNVFSCSVMHEALHNHYTQKSLSLSLG |
| 101 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGRTVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC |
| 102 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGDGLEWVAFMSYDGSNKKYADSV |
| | KGRFTISRDNSKNTLYLQMNSLRAEDTAVYYCARDRGIAAGGNYYYYGMDVWGQGTT |
| | VTVSSLGGGSGASTKGPSVFPLAPCSRSTSESTAALGCLVKDYFPEPVTVSWNSGALTSG |
| | VHTFPAVLQSSGLYSLSSVVTVPSSSLGTKTYTCNVDHKPSNTKVDKRVESKYGPPCPPC |
| | PAPEFLGGPSVFLFPPKPKDTLYITREPEVTCVVVDVSQEDPEVQFNWYVDGVEVHNAKT |
| | KPREEQFNSTYRVVSVLTVLHQDWLNGKEYKCKVSNKGLPSSIEKTISKAKGQPREPQVY |
| | TLPPSQEEMTKNQVSLTCLVKGFYPSDIAVEWESNGQPENNYKTTPPVLDSDGSFFLYSR |
| | LTVDKSRWQEGNVFSCSVMHEALHNHYTQKSLSLSLG |
| 103 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGRTVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC |
| 104 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGDGLEWVAFMSYDGSNKKYADSV |
| | KGRFTISRDNSKNTLYLQMNSLRAEDTAVYYCARDRGIAAGGNYYYYGMDVWGQGTT |
| | VTVSSFNRGESASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKRVEPKSCDKTHTC |
| | PPCPAPEAAGGPSVFLFPPKPKDTLMISRTPEVTCVVVDVSHEDPEVKFNWYVDGVEVHN |
| | AKTKPREEQYNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPG |
| 105 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSVEPK |
| | SSRTVAAPSVFIFPPSDEQLKSGTASVVCLLNNFYPREAKVQWKVDNALQSGNSQESVTE |
| | QDSKDSTYSLSSTLTLSKADYEKHKVYACEVTHQGLSSPVTKSFNRGEC |
| 106 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGDGLEWVAFMSYDGSNKKYADSV |
| | KGRFTISRDNSKNTLYLQMNSLRAEDTAVYYCARDRGIAAGGNYYYYGMDVWGQGTT |
| | VTVSSFNRGESASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKRVEPKSCDKTHTC |
| | PPCPAPELLGGPSVFLFPPKPKDTLMISRTPEVTCVVVDVSHEDPEVKFNWYVDGVEVHN |
| | AKTKPREEQYNSTYRVVSVLTVLHQDWLNGKEYKCKVSNKALPAPIEKTISKAKGQPRE |
| | PQVYTLPPSREEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPG |
| 107 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSVEPK |
| | SSRTVAAPSVFIFPPSDEQLKSGTASVVCLLNNFYPREAKVQWKVDNALQSGNSQESVTE |
| | QDSKDSTYSLSSTLTLSKADYEKHKVYACEVTHQGLSSPVTKSFNRGEC |
| 108 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGDGLEWVAFMSYDGSNKKYADSV |
| | KGRFTISRDNSKNTLYLQMNSLRAEDTAVYYCARDRGIAAGGNYYYYGMDVWGQGTT |
| | VTVSSFNRGESASTKGPSVFPLAPCSRSTSESTAALGCLVKDYFPEPVTVSWNSGALTSGV |
| | HTFPAVLQSSGLYSLSSVVTVPSSSLGTKTYTCNVDHKPSNTKVDKRVESKYGPPCPPCP |
| | APEFLGGPSVFLFPPKPKDTLMISRTPEVTCVVVDVSQEDPEVQFNWYVDGVEVHNAKT |
| | KPREEQFNSTYRVVSVLTVLHQDWLNGKEYKCKVSNKGLPSSIEKTISKAKGQPREPQVY |
| | TLPPSQEEMTKNQVSLTCLVKGFYPSDIAVEWESNGQPENNYKTTPPVLDSDGSFFLYSR |
| | LTVDKSRWQEGNVFSCSVMHEALHNHYTQKSLSLSLG |
| 109 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSVEPK |
| | SSRTVAAPSVFIFPPSDEQLKSGTASVVCLLNNFYPREAKVQWKVDNALQSGNSQESVTE |
| | QDSKDSTYSLSSTLTLSKADYEKHKVYACEVTHQGLSSPVTKSFNRGEC |
| 110 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGDGLEWVAFMSYDGSNKKYADSV |
| | KGRFTISRDNSKNTLYLQMNSLRAEDTAVYYCARDRGIAAGGNYYYYGMDVWGQGTT |
| | VTVSSFNRGESASTKGPSVFPLAPCSRSTSESTAALGCLVKDYFPEPVTVSWNSGALTSGV |
| | HTFPAVLQSSGLYSLSSVVTVPSSSLGTKTYTCNVDHKPSNTKVDKRVESKYGPPCPPCP |
| | APEFLGGPSVFLFPPKPKDTLYITREPEVTCVVVDVSQEDPEVQFNWYVDGVEVHNAKT |
| | KPREEQFNSTYRVVSVLTVLHQDWLNGKEYKCKVSNKGLPSSIEKTISKAKGQPREPQVY |
| | TLPPSQEEMTKNQVSLTCLVKGFYPSDIAVEWESNGQPENNYKTTPPVLDSDGSFFLYSR |
| | LTVDKSRWQEGNVFSCSVMHEALHNHYTQKSLSLSLG |
| 111 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSVEPK |
| | SSRTVAAPSVFIFPPSDEQLKSGTASVVCLLNNFYPREAKVQWKVDNALQSGNSQESVTE |
| | QDSKDSTYSLSSTLTLSKADYEKHKVYACEVTHQGLSSPVTKSFNRGEC |
| 112 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGASTKGPSVFPLAPSSKSTSGGTAALGCLVKDYFPEPVTVSWNSGALTSGVHTFPAVL |
| | QSSGLYSLSSVVTVPSSSLGTQTYICNVNHKPSNTKVDKRVEPKSCDKTHTCPPCPAPEAA |
| | GGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSFFLYSKLTVD |
| | KSRWQQGNVFSCSVMHEALHNHYTQKSLSLSPG |
| 113 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGNGLEWVAFMSYDGSNKKYADSV |
| | KGRFTISRDNSKNTLYLQMNSLRAEDTAVYYCARDRGIAAGGNYYYYGMDVWGQGTT |
| | VTVSSLGGGSGRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKSFNRGEC |
| 114 | DIQMTQSPSSLSASVGDRVTITCRASQGIRNYLAWYQQKPGKAPKLLIYAASTLQSGVPS |
| | RFSGSGSGTDFTLTISSLQPEDVATYYCQRYNRAPYTFGQGTKVEIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGASTKGPSVFPLAPSSKSTSGGTAALGCLVKDYFPEPVTVSWNSGALTSGVHTFPAVL |
| | QSSGLYSLSSVVTVPSSSLGTQTYICNVNHKPSNTKVDKRVEPKSCDKTHTCPPCPAPELL |
| | GGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSFFLYSKLTVD |
| | KSRWQQGNVFSCSVMHEALHNHYTQKSLSLSPG |
| 115 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGEVQLVE |
| | SGGGLVQPGRSLRLSCAASGFTFDDYAMHWVRQAPGKGLEWVSAITWNSGHIDYADSV |
| | EGRFTISRDNAKNSLYLQMNSLRAEDTAVYYCAKVSYLSTASSLDYWGQGTLVTVSSLG |
| | GGSGRTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| 116 | DIQMTQSPSSLSASVGDRVTITCRASQGIRNYLAWYQQKPGKAPKLLIYAASTLQSGVPS |
| | RFSGSGSGTDFTLTISSLQPEDVATYYCQRYNRAPYTFGQGTKVEIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGASTKGPSVFPLAPCSRSTSESTAALGCLVKDYFPEPVTVSWNSGALTSGVHTFPAVL |
| | QSSGLYSLSSVVTVPSSSLGTKTYTCNVDHKPSNTKVDKRVESKYGPPCPPCPAPEFLGGP |
| | SVFLFPPKPKDTLMISRTPEVTCVVVDVSQEDPEVQFNWYVDGVEVHNAKTKPREEQFN |
| | STYRVVSVLTVLHQDWLNGKEYKCKVSNKGLPSSIEKTISKAKGQPREPQVYTLPPSQEE |
| | MTKNQVSLTCLVKGFYPSDIAVEWESNGQPENNYKTTPPVLDSDGSFFLYSRLTVDKSR |
| | WQEGNVFSCSVMHEALHNHYTQKSLSLSLG |
| 117 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGEVQLVE |
| | SGGGLVQPGRSLRLSCAASGFTFDDYAMHWVRQAPGKGLEWVSAITWNSGHIDYADSV |
| | EGRFTISRDNAKNSLYLQMNSLRAEDTAVYYCAKVSYLSTASSLDYWGQGTLVTVSSLG |
| | GGSGRTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| 118 | DIQMTQSPSSLSASVGDRVTITCRASQGIRNYLAWYQQKPGKAPKLLIYAASTLQSGVPS |
| | RFSGSGSGTDFTLTISSLQPEDVATYYCQRYNRAPYTFGQGTKVEIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGASTKGPSVFPLAPCSRSTSESTAALGCLVKDYFPEPVTVSWNSGALTSGVHTFPAVL |
| | QSSGLYSLSSVVTVPSSSLGTKTYTCNVDHKPSNTKVDKRVESKYGPPCPPCPAPEFLGGP |
| | SVFLFPPKPKDTLYITREPEVTCVVVDVSQEDPEVQFNWYVDGVEVHNAKTKPREEQFNS |
| | TYRVVSVLTVLHQDWLNGKEYKCKVSNKGLPSSIEKTISKAKGQPREPQVYTLPPSQEEM |
| | TKNQVSLTCLVKGFYPSDIAVEWESNGQPENNYKTTPPVLDSDGSFFLYSRLTVDKSRW |
| | QEGNVFSCSVMHEALHNHYTQKSLSLSLG |
| 119 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGEVQLVE |
| | SGGGLVQPGRSLRLSCAASGFTFDDYAMHWVRQAPGKGLEWVSAITWNSGHIDYADSV |
| | EGRFTISRDNAKNSLYLQMNSLRAEDTAVYYCAKVSYLSTASSLDYWGQGTLVTVSSLG |
| | GGSGRTVAAPSVFIFPPSDEQLKSGTASVVCLLNNFYPREAKVQWKVDNALQSGNSQES |
| | VTEQDSKDSTYSLSSTLTLSKADYEKHKVYACEVTHQGLSSPVTKSFNRGEC |
| 120 | DIQMTQSPSSLSASVGDRVTITCRASQGIRNYLAWYQQKPGKAPKLLIYAASTLQSGVPS |
| | RFSGSGSGTDFTLTISSLQPEDVATYYCQRYNRAPYTFGQGTKVEIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSFNRG |
| | ESASTKGPSVFPLAPSSKSTSGGTAALGCLVKDYFPEPVTVSWNSGALTSGVHTFPAVLQ |
| | SSGLYSLSSVVTVPSSSLGTQTYICNVNHKPSNTKVDKRVEPKSCDKTHTCPPCPAPEAAG |
| | GPSVFLEPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLYSKLTVDKS |
| | RWQQGNVFSCSVMHEALHNHYTQKSLSLSPG |
| 121 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGEVQLVE |
| | SGGGLVQPGRSLRLSCAASGFTFDDYAMHWVRQAPGKGLEWVSAITWNSGHIDYADSV |
| | EGRFTISRDNAKNSLYLQMNSLRAEDTAVYYCAKVSYLSTASSLDYWGQGTLVTVSSVE |
| | PKSSRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 122 | DIQMTQSPSSLSASVGDRVTITCRASQGIRNYLAWYQQKPGKAPKLLIYAASTLQSGVPS |
| | RFSGSGSGTDFTLTISSLQPEDVATYYCQRYNRAPYTFGQGTKVEIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSFNRG |
| | ESASTKGPSVFPLAPSSKSTSGGTAALGCLVKDYFPEPVTVSWNSGALTSGVHTFPAVLQ |
| | SSGLYSLSSVVTVPSSSLGTQTYICNVNHKPSNTKVDKRVEPKSCDKTHTCPPCPAPELLG |
| | GPSVFLFPPKPKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFLYSKLTVDKS |
| | RWQQGNVFSCSVMHEALHNHYTQKSLSLSPG |
| 123 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGEVQLVE |
| | SGGGLVQPGRSLRLSCAASGFTFDDYAMHWVRQAPGKGLEWVSAITWNSGHIDYADSV |
| | EGRFTISRDNAKNSLYLQMNSLRAEDTAVYYCAKVSYLSTASSLDYWGQGTLVTVSSVE |
| | PKSSRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 124 | DIQMTQSPSSLSASVGDRVTITCRASQGIRNYLAWYQQKPGKAPKLLIYAASTLQSGVPS |
| | RFSGSGSGTDFTLTISSLQPEDVATYYCQRYNRAPYTFGQGTKVEIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSFNRG |
| | ESASTKGPSVFPLAPCSRSTSESTAALGCLVKDYFPEPVTVSWNSGALTSGVHTFPAVLQS |
| | SGLYSLSSVVTVPSSSLGTKTYTCNVDHKPSNTKVDKRVESKYGPPCPPCPAPEFLGGPSV |
| | FLFPPKPKDTLMISRTPEVTCVVVDVSQEDPEVQFNWYVDGVEVHNAKTKPREEQFNST |
| | YRVVSVLTVLHQDWLNGKEYKCKVSNKGLPSSIEKTISKAKGQPREPQVYTLPPSQEEMT |
| | KNQVSLTCLVKGFYPSDIAVEWESNGQPENNYKTTPPVLDSDGSFFLYSRLTVDKSRWQ |
| | EGNVFSCSVMHEALHNHYTQKSLSLSLG |
| 125 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGEVQLVE |
| | SGGGLVQPGRSLRLSCAASGFTFDDYAMHWVRQAPGKGLEWVSAITWNSGHIDYADSV |
| | EGRFTISRDNAKNSLYLQMNSLRAEDTAVYYCAKVSYLSTASSLDYWGQGTLVTVSSVE |
| | PKSSRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 126 | DIQMTQSPSSLSASVGDRVTITCRASQGIRNYLAWYQQKPGKAPKLLIYAASTLQSGVPS |
| | RFSGSGSGTDFTLTISSLQPEDVATYYCQRYNRAPYTFGQGTKVEIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSFNRG |
| | ESASTKGPSVFPLAPCSRSTSESTAALGCLVKDYFPEPVTVSWNSGALTSGVHTFPAVLQS |
| | SGLYSLSSVVTVPSSSLGTKTYTCNVDHKPSNTKVDKRVESKYGPPCPPCPAPEFLGGPSV |
| | FLFPPKPKDTLYITREPEVTCVVVDVSQEDPEVQFNWYVDGVEVHNAKTKPREEQFNST |
| | YRVVSVLTVLHQDWLNGKEYKCKVSNKGLPSSIEKTISKAKGQPREPQVYTLPPSQEEMT |
| | KNQVSLTCLVKGFYPSDIAVEWESNGQPENNYKTTPPVLDSDGSFFLYSRLTVDKSRWQ |
| | EGNVFSCSVMHEALHNHYTQKSLSLSLG |
| 127 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGEVQLVE |
| | SGGGLVQPGRSLRLSCAASGFTFDDYAMHWVRQAPGKGLEWVSAITWNSGHIDYADSV |
| | EGRFTISRDNAKNSLYLQMNSLRAEDTAVYYCAKVSYLSTASSLDYWGQGTLVTVSSVE |
| | PKSSRTVAAPSVFIFPPSDEQLKSGTASVVCLLNNFYPREAKVQWKVDNALQSGNSQESV |
| | TEQDSKDSTYSLSSTLTLSKADYEKHKVYACEVTHQGLSSPVTKSFNRGEC |
| 128 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGEVQLVE |
| | SGGGLVQPGRSLRLSCAASGFTFDDYAMHWVRQAPGKGLEWVSAITWNSGHIDYADSV |
| | EGRFTISRDNAKNSLYLQMNSLRAEDTAVYYCAKVSYLSTASSLDYWGQGTLVTVSSLG |
| | GGSGASTKGPSVFPLAPSSKSTSGGTAALGCLVKDYFPEPVTVSWNSGALTSGVHTFPAV |
| | LQSSGLYSLSSVVTVPSSSLGTQTYICNVNHKPSNTKVDKRVEPKSCDKTHTCPPCPAPEA |
| | AGGPSVFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPG |
| 129 | DIQMTQSPSSLSASVGDRVTITCRASQGIRNYLAWYQQKPGKAPKLLIYAASTLQSGVPS |
| | RFSGSGSGTDFTLTISSLQPEDVATYYCQRYNRAPYTFGQGTKVEIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGRTVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC |
| 130 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGEVQLVE |
| | SGGGLVQPGRSLRLSCAASGFTFDDYAMHWVRQAPGKGLEWVSAITWNSGHIDYADSV |
| | EGRFTISRDNAKNSLYLQMNSLRAEDTAVYYCAKVSYLSTASSLDYWGQGTLVTVSSLG |
| | GGSGASTKGPSVFPLAPSSKSTSGGTAALGCLVKDYFPEPVTVSWNSGALTSGVHTFPAV |
| | LQSSGLYSLSSVVTVPSSSLGTQTYICNVNHKPSNTKVDKRVEPKSCDKTHTCPPCPAPEL |
| | LGGPSVFLFPPKPKDTLMISRTPEVTCVVVDVSHEDPEVKFNWYVDGVEVHNAKTKPRE |
| | EQYNSTYRVVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPG |
| 131 | DIQMTQSPSSLSASVGDRVTITCRASQGIRNYLAWYQQKPGKAPKLLIYAASTLQSGVPS |
| | RFSGSGSGTDFTLTISSLQPEDVATYYCQRYNRAPYTFGQGTKVEIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGRTVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC |
| 132 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGEVQLVE |
| | SGGGLVQPGRSLRLSCAASGFTFDDYAMHWVRQAPGKGLEWVSAITWNSGHIDYADSV |
| | EGRFTISRDNAKNSLYLQMNSLRAEDTAVYYCAKVSYLSTASSLDYWGQGTLVTVSSLG |
| | GGSGASTKGPSVFPLAPCSRSTSESTAALGCLVKDYFPEPVTVSWNSGALTSGVHTFPAV |
| | LQSSGLYSLSSVVTVPSSSLGTKTYTCNVDHKPSNTKVDKRVESKYGPPCPPCPAPEFLGG |
| | PSVFLFPPKPKDTLMISRTPEVTCVVVDVSQEDPEVQFNWYVDGVEVHNAKTKPREEQF |
| | NSTYRVVSVLTVLHQDWLNGKEYKCKVSNKGLPSSIEKTISKAKGQPREPQVYTLPPSQE |
| | EMTKNQVSLTCLVKGFYPSDIAVEWESNGQPENNYKTTPPVLDSDGSFFLYSRLTVDKSR |
| | WQEGNVFSCSVMHEALHNHYTQKSLSLSLG |
| 133 | DIQMTQSPSSLSASVGDRVTITCRASQGIRNYLAWYQQKPGKAPKLLIYAASTLQSGVPS |
| | RFSGSGSGTDFTLTISSLQPEDVATYYCQRYNRAPYTFGQGTKVEIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGRTVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC |
| 134 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGEVQLVE |
| | SGGGLVQPGRSLRLSCAASGFTFDDYAMHWVRQAPGKGLEWVSAITWNSGHIDYADSV |
| | EGRFTISRDNAKNSLYLQMNSLRAEDTAVYYCAKVSYLSTASSLDYWGQGTLVTVSSLG |
| | GGSGASTKGPSVFPLAPCSRSTSESTAALGCLVKDYFPEPVTVSWNSGALTSGVHTFPAV |
| | LQSSGLYSLSSVVTVPSSSLGTKTYTCNVDHKPSNTKVDKRVESKYGPPCPPCPAPEFLGG |
| | PSVFLFPPKPKDTLYITREPEVTCVVVDVSQEDPEVQFNWYVDGVEVHNAKTKPREEQFN |
| | STYRVVSVLTVLHQDWLNGKEYKCKVSNKGLPSSIEKTISKAKGQPREPQVYTLPPSQEE |
| | MTKNQVSLTCLVKGFYPSDIAVEWESNGQPENNYKTTPPVLDSDGSFFLYSRLTVDKSR |
| | WQEGNVFSCSVMHEALHNHYTQKSLSLSLG |
| 135 | DIQMTQSPSSLSASVGDRVTITCRASQGIRNYLAWYQQKPGKAPKLLIYAASTLQSGVPS |
| | RFSGSGSGTDFTLTISSLQPEDVATYYCQRYNRAPYTFGQGTKVEIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGRTVAAPSVFIFPPSDEQLKSGTASVVCLLNNFYPREAKVQWKVDNALQSGNSQESVT |
| | EQDSKDSTYSLSSTLTLSKADYEKHKVYACEVTHQGLSSPVTKSFNRGEC |
| 136 | EIVLTQSPATLSLSPGERATLSCRASQSVYSYLAWYQQKPGQAPRLLIYDASNRATGIPAR |
| | FSGSGSGTDFTLTISSLEPEDFAVYYCQQRSNWPPFTFGPGTKVDIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSLGG |
| | GSGASTKGPSVFPLAPSSKSTSGGTAALGCLVKDYFPEPVTVSWNSGALTSGVHTFPAVL |
| | QSSGLYSLSSVVTVPSSSLGTQTYICNVNHKPSNTKVDKRVEPKSCDKTHTCPPCPAPELL |
| | GGPSVFLFPPKPKDTLMISRTPEVTCVVVDVSHEDPEVKFNWYVDGVEVHNAKTKPREE |
| | QYNSTYRVVSVLTVLHQDWLNGKEYKCKVSNKALPAPIEKTISKAKGQPREPQVYTLPP |
| | SREEMTKNQVSLTCLVKGFYPSDIAVEWESNGQPENNYKTTPPVLDSDGSFFLYSKLTVD |
| | KSRWQQGNVFSCSVMHEALHNHYTQKSLSLSPG |
| 137 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGQVQLVE |
| | SGGGVVQPGRSLRLSCAASGFIFSSYAMHWVRQAPGNGLEWVAFMSYDGSNKKYADSV |
| | KGRFTISRDNSKNTLYLQMNSLRAEDTAVYYCARDRGIAAGGNYYYYGMDVWGQGTT |
| | VTVSSLGGGSGRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKSFNRGEC |
| 138 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGEVQLVE |
| | SGGGLVQPGRSLRLSCAASGFTFDDYAMHWVRQAPGKGLEWVSAITWNSGHIDYADSV |
| | EGRFTISRDNAKNSLYLQMNSLRAEDTAVYYCAKVSYLSTASSLDYWGQGTLVTVSSFN |
| | RGESASTKGPSVFPLAPSSKSTSGGTAALGCLVKDYFPEPVTVSWNSGALTSGVHTFPAV |
| | LQSSGLYSLSSVVTVPSSSLGTQTYICNVNHKPSNTKVDKRVEPKSCDKTHTCPPCPAPEL |
| | LGGPSVFLFPPKPKDTLMISRTPEVTCVVVDVSHEDPEVKFNWYVDGVEVHNAKTKPRE |
| | FQYNSTYRVVSVLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDSDGSFFLYSKLTV |
| | DKSRWQQGNVFSCSVMHEALHNHYTQKSLSLSPG |
| 139 | DIQMTQSPSSLSASVGDRVTITCRASQGIRNYLAWYQQKPGKAPKLLIYAASTLQSGVPS |
| | RFSGSGSGTDFTLTISSLQPEDVATYYCQRYNRAPYTFGQGTKVEIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSVEPK |
| | SSRTVAAPSVFIFPPSDEQLKSGTASVVCLLNNFYPREAKVQWKVDNALQSGNSQESVTE |
| | QDSKDSTYSLSSTLTLSKADYEKHKVYACEVTHQGLSSPVTKSFNRGEC |
| 140 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGEVQLVE |
| | SGGGLVQPGRSLRLSCAASGFTFDDYAMHWVRQAPGKGLEWVSAITWNSGHIDYADSV |
| | EGRFTISRDNAKNSLYLQMNSLRAEDTAVYYCAKVSYLSTASSLDYWGQGTLVTVSSFN |
| | RGESASTKGPSVFPLAPCSRSTSESTAALGCLVKDYFPEPVTVSWNSGALTSGVHTFPAVL |
| | QSSGLYSLSSVVTVPSSSLGTKTYTCNVDHKPSNTKVDKRVESKYGPPCPPCPAPEFLGGP |
| | SVFLFPPKPKDTLMISRTPEVTCVVVDVSQEDPEVQFNWYVDGVEVHNAKTKPREEQFN |
| | STYRVVSVLTVLHQDWLNGKEYKCKVSNKGLPSSIEKTISKAKGQPREPQVYTLPPSQEE |
| | MTKNQVSLTCLVKGFYPSDIAVEWESNGQPENNYKTTPPVLDSDGSFFLYSRLTVDKSR |
| | WQEGNVFSCSVMHEALHNHYTQKSLSLSLG |
| 141 | DIQMTQSPSSLSASVGDRVTITCRASQGIRNYLAWYQQKPGKAPKLLIYAASTLQSGVPS |
| | RFSGSGSGTDFTLTISSLQPEDVATYYCQRYNRAPYTFGQGTKVEIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSVEPK |
| | SSRTVAAPSVFIFPPSDEQLKSGTASVVCLLNNFYPREAKVQWKVDNALQSGNSQESVTE |
| | QDSKDSTYSLSSTLTLSKADYEKHKVYACEVTHQGLSSPVTKSFNRGEC |
| 142 | DIQMTQSPSSLSASVGDRVTITCKASRDVAIAVAWYQQKPGKVPKLLIYWASTRHTGVPS |
| | RFSGSGSRTDFTLTISSLQPEDVADYFCHQYSSYPFTFGSGTKLEIKGGGSGGGGEVQLVE |
| | SGGGLVQPGRSLRLSCAASGFTFDDYAMHWVRQAPGKGLEWVSAITWNSGIHDYADSV |
| | EGRFTISRDNAKNSLYLQMNSLRAEDTAVYYCAKVSYLSTASSLDYWGQGTLVTVSSFN |
| | RGESASTKGPSVFPLAPCSRSTSESTAALGCLVKDYFPEPVTVSWNSGALTSGVHTFPAVL |
| | QSSGLYSLSSVVTVPSSSLGTKTYTCNVDHKPSNTKVDKRVESKYGPPCPPCPAPEFLGGP |
| | SVFLFPPKPKDTLYITREPEVTCVVVDVSQEDPEVQFNWYVDGVEVHNAKTKPREEQFNS |
| | TYRVVSVLTVLHQDWLNGKEYKCKVSNKGLPSSIEKTISKAKGQPREPQVYTLPPSQEEM |
| | TKNQVSLTCLVKGFYPSDIAVEWESNGQPENNYKTTPPVLDSDGSFFLYSRLTVDKSRW |
| | QEGNVFSCSVMHEALHNHYTQKSLSLSLG |
| 143 | DIQMTQSPSSLSASVGDRVTITCRASQGIRNYLAWYQQKPGKAPKLLIYAASTLQSGVPS |
| | RFSGSGSGTDFTLTISSLQPEDVATYYCQRYNRAPYTFGQGTKVEIKGGGSGGGGQVQLV |
| | QSGAEVKKPGSSVKVSCKASGYTFTDQTIHWMRQAPGQGLEWIGYIYPRDDSPKYNENF |
| | KGKVTITADKSTSTAYMELSSLRSEDTAVYYCAIPDRSGYAWFIYWGQGTLVTVSSVEPK |
| | SSRTVAAPSVFIFPPSDEQLKSGTASVVCLLNNFYPREAKVQWKVDNALQSGNSQESVTE |
| | QDSKDSTYSLSSTLTLSKADYEKHKVYACEVTHQGLSSPVTKSFNRGEC |
| 144 | MSTESMIRDVELAEEALPKKTGGPQGSRRCLFLSLFSFLIVAGATTLFCLLHFGVIGPQREE |
| | FPRDLSLISPLAQAVRSSSRTPSDKPVAHVVANPQAEGQLQWLNRRANALLANGVELRD |
| | NQLVVPSEGLYLIYSQVLFKGQGCPSTHVLLTHTISRIAVSYQTKVNLLSAIKSPCQRETPE |
| | GAEAKPWYEPIYLGGVFQLEKGDRLSAEINRPDYLDFAESGQVYFGHAL |
| 145 | MLGSRAVMLLLLLPWTAQGRAVPGGSSPAWTQCQQLSQKLCTLAWSAHPLVGHMDLR |
| | EEGDEETTNDVPHIQCGDGCDPQGLRDNSQFCLQRIHQGLIFYEKLLGSDIFTGEPSLLPDS |
| | PVGQLHASLLGLSQLLQPEGHHWETQQIPSLSPSQPWQRLLLRFKILRSLQAFVAVAARV |
| | FAHGAATLSP |
| 146 | DYAMH |
| 147 | AITWNSGHIDYADSVEG |
| 148 | VSYLSTASSLDY |
| 149 | RASQGIRNYLA |
| 150 | AASTLQS |
| 151 | QRYNRAPYT |
| 152 | SYAMH |
| 153 | FMSYDGSNKKYADSVKG |
| 154 | NYYYYGMDV |
| 155 | RASQSVYSYLA |
| 156 | DASNRAT |
| 157 | QQRSNWPPFT |
| 158 | DQTIH |
| 159 | YIYPRDDSPKYNENFKG |
| 160 | PDRSGYAWFIY |
| 161 | KASRDVAIAVA |
| 162 | WASTRHT |
| 163 | HQYSSYPFT |

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

What is claimed is:

1. A compound comprising a first polypeptide and a second polypeptide, wherein:
(A) said first polypeptide comprises:
(i) a light chain variable domain of a first immunoglobulin (VL1) specific for a first target protein;
(ii) a heavy chain variable domain of a second immunoglobulin (VH2) specific for a second target protein; and
(iii) a hinge region, a heavy chain constant region 2 (CH2) and a heavy chain constant region 3 (CH3); and
(B) said second polypeptide comprises:
(i) a light chain variable domain of the second immunoglobulin (VL2) specific for said second target protein;
(ii) a heavy chain variable domain of the first immunoglobulin (VH1) specific for said first target protein;
wherein:
(i) said VL1 and VH1 associate to form a binding site that binds said first target protein;
(ii) said VL2 and VH2 associate to form a binding site that binds said second target protein;
(iii) said heavy chain constant region 2 (CH2) comprises a tyrosine at position 252, a threonine at position 254 and a glutamic acid a position 256, numbered according to the EU index as in Kabat; and
(iv) said first target protein is TNF-alpha and said second target protein is IL-23A or said first target protein is IL-23A and said second target protein is TNF-alpha,
wherein:
(i) said VL1 comprises SEQ ID NO:2, said VH1 comprises SEQ ID NO: 1, said VL2 comprises SEQ ID NO:8 and said VH2 comprises SEQ ID NO:7; or
(ii) said VL1 comprises SEQ ID NO:4 or 6, said VH1 comprises SEQ ID NO:3 or 5, said VL2 comprises SEQ ID NO:8 and said VH2 comprises SEQ ID NO:7; or
(iii) said VL1 comprises SEQ ID NO:8, said VH1 comprises SEQ ID NO:7, said VL2 comprises SEQ ID NO:2 and said VH2 comprises SEQ ID NO: 1; or
(iv) said VL1 comprises SEQ ID NO:8, said VH1 comprises SEQ ID NO:7, said VL2 comprises SEQ ID NO:4 or 6 and said VH2 comprises SEQ ID NO:3 or 5.

2-5. (canceled)

6. The compound according to claim 1, wherein said first polypeptide further comprises a first linker between said VL1 and said VH2 and said second polypeptide further comprises a second linker between said VL2 and said VH1.

7. The compound according to claim 6, wherein said first linker id/or said second linker comprises the amino acid sequence GGGSGGG (SEQ ID NO:9).

8. (canceled)

9. The compound according to claim 1, wherein said first polypeptide further comprises a heavy chain constant region 1 domain (CH1) and said second polypeptide further comprises a light chain constant region domain (CL), wherein said CL and said CH1 are associated together via a disulfide bond to form a C1 domain.

10. The compound according to claim 9, wherein said first polypeptide further comprises a third linker between said VH2 and said CH1 and said second polypeptide further comprises a fourth linker between said VH1 and said CL.

11. The compound according to claim 10, wherein said third linker comprises the amino acid sequence FNRGES (SEQ ID NO: 11) and/or said fourth linker comprises the amino acid sequence VEPKSS (SEO ID NO: 12).

12-13. (canceled)

14. The compound according to claim 10, wherein said third linker and/or said fourth linker comprises the amino acid sequence LGGGSG (SEQ ID NO: 10).

15. (canceled)

16. The compound according to claim 1, wherein said heavy chain constant region 2 (CH2) comprises an alanine at positions 234 and an alanine at position 235, numbered according to the EU index as in Kabat.

17. The compound according to claim 1, wherein the amino acid sequence of said hinge region, said heavy chain constant region 2 (CH2) or said heavy chain constant region 3 (CH3) is derived from a IgG1 or from a IgG4, wherein optionally said hinge region comprises the amino acid sequence EPKSCDKTHTCPPCP (SEQ ID NO:40).

18. (canceled)

19. The compound according to claim 1, wherein said compound comprises two said first polypeptides and two said second polypeptides, wherein said two first polypeptides are associated together via at least one disulfide bond.

20. The compound according to claim 1, wherein:
(i) said first polypeptide comprises the amino acid sequence of SEQ ID NO: 13 and said second polypeptide comprises the amino acid sequence of SEQ ID NO: 14;
(ii) said first polypeptide comprises the amino acid sequence of SEQ ID NO: 15 and said second polypeptide comprises the amino acid sequence of SEQ ID NO: 16;
(iii) said first polypeptide comprises the amino acid sequence of SEQ ID NO: 17 and said second polypeptide comprises the amino acid sequence of SEQ ID NO: 18;
(iv) said first polypeptide comprises the amino acid sequence of SEQ ID NO: 19 and said second polypeptide comprises the amino acid sequence of SEQ ID NO:20;
(v) said first polypeptide comprises the amino acid sequence of SEQ ID NO:21 and said second polypeptide comprises the amino acid sequence of SEQ ID NO:22;
(vi) said first polypeptide comprises the amino acid sequence of SEQ ID NO:23 and said second polypeptide comprises the amino acid sequence of SEQ ID NO:24;
(vii) said first polypeptide comprises the amino acid sequence of SEQ ID NO:25 and said second polypeptide comprises the amino acid sequence of SEQ ID NO:26;
(viii) said first polypeptide comprises the amino acid sequence of SEQ ID NO:27 and said second polypeptide comprises the amino acid sequence of SEQ ID NO:28;
(ix) said first polypeptide comprises the amino acid sequence of SEQ ID NO:29 and said second polypeptide comprises the amino acid sequence of SEQ ID NO:30;
(x) said first polypeptide comprises the amino acid sequence of SEQ ID NO:31 and said second polypeptide comprises the amino acid sequence of SEQ ID NO:32;
(xi) said first polypeptide comprises the amino acid sequence of SEQ ID NO:33 and said second polypeptide comprises the amino acid sequence of SEQ ID NO:34; or
(xii) said first polypeptide comprises the amino acid sequence of SEQ ID NO:35 and said second polypeptide comprises the amino acid sequence of SEQ ID NO:36.

21. The compound according to claim 20, wherein said compound comprises two said first polypeptides and two said second polypeptides, wherein said two first polypeptides are associated together via at least one disulfide bond and wherein each of said first polypeptide is associated to one said second polypeptide via at least one disulfide bond and optionally said compound comprises two said first polypeptides and two said second polypeptides, wherein each of said first polypeptides comprises a CH1, a CH2 and a CH3 and each of said second polypeptides comprises a CL and wherein the CH2 and CH3 of one of the first polypeptides associates with the CH2 and CH3 of the other of the first polypeptides and the CH1 of each said first polypeptides associates with the CL of one said second polypeptides to form a tetravalent molecule.

22-23. (canceled)

24. A pharmaceutical composition comprising a compound according to claim 1.

25. A method of treating an autoimmune or an inflammatory disease comprising administering a compound according to claim 1 or a pharmaceutical composition comprising said compound to a subject.

26-29. (canceled)

30. A nucleic acid comprising a nucleotide sequence encoding a compound according to claim 1.

31. A vector comprising a nucleic acid according to claim 30, optionally comprising a promoter operably linked to said nucleic acid.

32. (canceled)

33. A cell comprising a nucleic acid according to claim 30 or a vector comprising said nucleic acid.

34. A method of producing a compound according to claim 1 comprising obtaining a cell comprising a nucleic acid encoding said compound and expressing said nucleic acid in said cell, and optionally isolating and purifying said compound.

35. (canceled)


SEQUENCE LISTING (163 sequences)

SEQ ID NO: 1 - protein, 121 aa, synthetic construct [sequence omitted]

SEQ ID NO: 2 - protein, 107 aa, synthetic construct [sequence omitted]

SEQ ID NO: 3 - protein, 126 aa, synthetic construct
  REGION 1..126: note = Synthetic Polypeptide
QVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSS

SEQ ID NO: 4 - protein, 108 aa, synthetic construct
  REGION 1..108: note = Synthetic Polypeptide
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIK

SEQ ID NO: 5 - protein, 126 aa, synthetic construct
  REGION 1..126: note = Synthetic Polypeptide
QVQLVESGGGVVQPGRSLRLSCAASGFIFSSYAMHWVRQAPGDGLEWVAFMSYDGSNKKYADSVKGRFTISRDNSKNTLYLQMNSLRAEDTAVYYCARDRGIAAGGNYYYYGMDVWGQGTTVTVSS

SEQ ID NO: 6 - protein, 108 aa, synthetic construct; identical to SEQ ID NO: 4
  REGION 1..108: note = Synthetic Polypeptide

SEQ ID NO: 7 - protein, 120 aa, synthetic construct
  REGION 1..120: note = Synthetic Polypeptide
QVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSS

SEQ ID NO: 8 - protein, 107 aa, synthetic construct
  REGION 1..107: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIK

SEQ ID NO: 9 - protein, 7 aa, synthetic construct
  REGION 1..7: note = Synthetic Polypeptide
GGGSGGG

SEQ ID NO: 10 - protein, 6 aa, synthetic construct
  REGION 1..6: note = Synthetic Polypeptide
LGGGSG

SEQ ID NO: 11 - protein, 6 aa, synthetic construct
  REGION 1..6: note = Synthetic Polypeptide
FNRGES

SEQ ID NO: 12 - protein, 6 aa, synthetic construct
  REGION 1..6: note = Synthetic Polypeptide
VEPKSS

SEQ ID NO: 13 - protein, 571 aa, synthetic construct
  REGION 1..571: note = Synthetic Polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKGGGSGGGGQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSLGGGSGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 349 aa, synthetic construct
  REGION 1..349: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSLGGGSGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 571 aa, synthetic construct
  REGION 1..571: note = Synthetic Polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKGGGSGGGGQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSFNRGESASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 349 aa, synthetic construct
  REGION 1..349: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSVEPKSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 572 aa, synthetic construct
  REGION 1..572: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSLGGGSGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 348 aa, synthetic construct
  REGION 1..348: note = Synthetic Polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKGGGSGGGGQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSLGGGSGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 572 aa, synthetic construct
  REGION 1..572: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSFNRGESASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 348 aa, synthetic construct
  REGION 1..348: note = Synthetic Polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKGGGSGGGGQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSVEPKSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 577 aa, synthetic construct
  REGION 1..577: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGQVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSLGGGSGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 349 aa, synthetic construct
  REGION 1..349: note = Synthetic Polypeptide
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKGGGSGGGGQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSLGGGSGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 577 aa, synthetic construct
  REGION 1..577: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGQVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSFNRGESASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 349 aa, synthetic construct
  REGION 1..349: note = Synthetic Polypeptide
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKGGGSGGGGQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSVEPKSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 572 aa, synthetic construct
  REGION 1..572: note = Synthetic Polypeptide
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKGGGSGGGGQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSLGGGSGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 354 aa, synthetic construct
  REGION 1..354: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGQVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSLGGGSGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 572 aa, synthetic construct
  REGION 1..572: note = Synthetic Polypeptide
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKGGGSGGGGQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSFNRGESASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 354 aa, synthetic construct
  REGION 1..354: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGQVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSVEPKSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 572 aa, synthetic construct; identical to SEQ ID NO: 25
  REGION 1..572: note = Synthetic Polypeptide

SEQ ID NO: 30 - protein, 354 aa, synthetic construct
  REGION 1..354: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGQVQLVESGGGVVQPGRSLRLSCAASGFIFSSYAMHWVRQAPGDGLEWVAFMSYDGSNKKYADSVKGRFTISRDNSKNTLYLQMNSLRAEDTAVYYCARDRGIAAGGNYYYYGMDVWGQGTTVTVSSLGGGSGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 577 aa, synthetic construct
  REGION 1..577: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGQVQLVESGGGVVQPGRSLRLSCAASGFIFSSYAMHWVRQAPGDGLEWVAFMSYDGSNKKYADSVKGRFTISRDNSKNTLYLQMNSLRAEDTAVYYCARDRGIAAGGNYYYYGMDVWGQGTTVTVSSLGGGSGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 349 aa, synthetic construct; identical to SEQ ID NO: 22
  REGION 1..349: note = Synthetic Polypeptide

SEQ ID NO: 33 - protein, 572 aa, synthetic construct; identical to SEQ ID NO: 27
  REGION 1..572: note = Synthetic Polypeptide

SEQ ID NO: 34 - protein, 354 aa, synthetic construct
  REGION 1..354: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGQVQLVESGGGVVQPGRSLRLSCAASGFIFSSYAMHWVRQAPGDGLEWVAFMSYDGSNKKYADSVKGRFTISRDNSKNTLYLQMNSLRAEDTAVYYCARDRGIAAGGNYYYYGMDVWGQGTTVTVSSVEPKSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 577 aa, synthetic construct
  REGION 1..577: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGQVQLVESGGGVVQPGRSLRLSCAASGFIFSSYAMHWVRQAPGDGLEWVAFMSYDGSNKKYADSVKGRFTISRDNSKNTLYLQMNSLRAEDTAVYYCARDRGIAAGGNYYYYGMDVWGQGTTVTVSSFNRGESASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 349 aa, synthetic construct; identical to SEQ ID NO: 24
  REGION 1..349: note = Synthetic Polypeptide

SEQ ID NO: 37 - protein, 326 aa, synthetic construct
  REGION 1..326: note = Synthetic Polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 38 - protein, 326 aa, synthetic construct
  REGION 1..326: note = Synthetic Polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 39 - protein, 329 aa, synthetic construct
  REGION 1..329: note = Synthetic Polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 15 aa, synthetic construct
  REGION 1..15: note = Synthetic Polypeptide
EPKSCDKTHTCPPCP

SEQ ID NO: 41 - protein, 107 aa, synthetic construct [sequence omitted]

SEQ ID NO: 42 - protein, 330 aa, synthetic construct
  REGION 1..330: note = Synthetic Polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 329 aa, synthetic construct
  REGION 1..329: note = Synthetic Polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 570 aa, synthetic construct
  REGION 1..570: note = Synthetic Polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKGGGSGGGGQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSLGGGSGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 45 - protein, 349 aa, synthetic construct; identical to SEQ ID NO: 14
  REGION 1..349: note = Synthetic Polypeptide

SEQ ID NO: 46 - protein, 571 aa, synthetic construct
  REGION 1..571: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSFNRGESASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - protein, 348 aa, synthetic construct; identical to SEQ ID NO: 20
  REGION 1..348: note = Synthetic Polypeptide

SEQ ID NO: 48 - protein, 576 aa, synthetic construct
  REGION 1..576: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGQVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSLGGGSGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - protein, 349 aa, synthetic construct; identical to SEQ ID NO: 22
  REGION 1..349: note = Synthetic Polypeptide

SEQ ID NO: 50 - protein, 576 aa, synthetic construct
  REGION 1..576: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGQVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSFNRGESASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 51 - protein, 349 aa, synthetic construct; identical to SEQ ID NO: 24
  REGION 1..349: note = Synthetic Polypeptide

SEQ ID NO: 52 - protein, 568 aa, synthetic construct
  REGION 1..568: note = Synthetic Polypeptide
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKGGGSGGGGQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSLGGGSGASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 53 - protein, 354 aa, synthetic construct; identical to SEQ ID NO: 26
  REGION 1..354: note = Synthetic Polypeptide

SEQ ID NO: 54 - protein, 568 aa, synthetic construct
  REGION 1..568: note = Synthetic Polypeptide
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKGGGSGGGGQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSLGGGSGASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 55 - protein, 354 aa, synthetic construct; identical to SEQ ID NO: 26
  REGION 1..354: note = Synthetic Polypeptide

SEQ ID NO: 56 - protein, 568 aa, synthetic construct; identical to SEQ ID NO: 52
  REGION 1..568: note = Synthetic Polypeptide

SEQ ID NO: 57 - protein, 354 aa, synthetic construct; identical to SEQ ID NO: 26
  REGION 1..354: note = Synthetic Polypeptide

SEQ ID NO: 58 - protein, 568 aa, synthetic construct; identical to SEQ ID NO: 54
  REGION 1..568: note = Synthetic Polypeptide

SEQ ID NO: 59 - protein, 354 aa, synthetic construct; identical to SEQ ID NO: 26
  REGION 1..354: note = Synthetic Polypeptide

SEQ ID NO: 60 - protein, 571 aa, synthetic construct
  REGION 1..571: note = Synthetic Polypeptide
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKGGGSGGGGQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSFNRGESASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 61 - protein, 354 aa, synthetic construct; identical to SEQ ID NO: 28
  REGION 1..354: note = Synthetic Polypeptide

SEQ ID NO: 62 - protein, 571 aa, synthetic construct
  REGION 1..571: note = Synthetic Polypeptide
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKGGGSGGGGQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSFNRGESASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 63 - protein, 354 aa, synthetic construct; identical to SEQ ID NO: 28
  REGION 1..354: note = Synthetic Polypeptide

SEQ ID NO: 64 - protein, 568 aa, synthetic construct
  REGION 1..568: note = Synthetic Polypeptide
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKGGGSGGGGQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSFNRGESASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 65 - protein, 354 aa, synthetic construct; identical to SEQ ID NO: 28
  REGION 1..354: note = Synthetic Polypeptide

SEQ ID NO: 66 - protein, 568 aa, synthetic construct
  REGION 1..568: note = Synthetic Polypeptide
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKGGGSGGGGQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSFNRGESASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 67 - protein, 354 aa, synthetic construct; identical to SEQ ID NO: 28
  REGION 1..354: note = Synthetic Polypeptide

SEQ ID NO: 68 - protein, 576 aa, synthetic construct
  REGION 1..576: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGQVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSLGGGSGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 69 - protein, 349 aa, synthetic construct; identical to SEQ ID NO: 22
  REGION 1..349: note = Synthetic Polypeptide

SEQ ID NO: 70 - protein, 573 aa, synthetic construct
  REGION 1..573: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGQVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSLGGGSGASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 71 - protein, 349 aa, synthetic construct; identical to SEQ ID NO: 22
  REGION 1..349: note = Synthetic Polypeptide

SEQ ID NO: 72 - protein, 573 aa, synthetic construct
  REGION 1..573: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGQVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSLGGGSGASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 73 - protein, 349 aa, synthetic construct; identical to SEQ ID NO: 22
  REGION 1..349: note = Synthetic Polypeptide

SEQ ID NO: 74 - protein, 576 aa, synthetic construct
  REGION 1..576: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGQVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSFNRGESASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 75 - protein, 349 aa, synthetic construct; identical to SEQ ID NO: 24
  REGION 1..349: note = Synthetic Polypeptide

SEQ ID NO: 76 - protein, 573 aa, synthetic construct
  REGION 1..573: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGQVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSFNRGESASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 77 - protein, 349 aa, synthetic construct; identical to SEQ ID NO: 24
  REGION 1..349: note = Synthetic Polypeptide

SEQ ID NO: 78 - protein, 573 aa, synthetic construct
  REGION 1..573: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGQVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSFNRGESASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 79 - protein, 349 aa, synthetic construct; identical to SEQ ID NO: 24
  REGION 1..349: note = Synthetic Polypeptide

SEQ ID NO: 80 - protein, 571 aa, synthetic construct
  REGION 1..571: note = Synthetic Polypeptide
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKGGGSGGGGQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSLGGGSGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 81 - protein, 354 aa, synthetic construct; identical to SEQ ID NO: 30
  REGION 1..354: note = Synthetic Polypeptide

SEQ ID NO: 82 - protein, 571 aa, synthetic construct
  REGION 1..571: note = Synthetic Polypeptide
EIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKGGGSGGGGQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSLGGGSGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 83 - protein, 354 aa, synthetic construct; identical to SEQ ID NO: 30
  REGION 1..354: note = Synthetic Polypeptide

SEQ ID NO: 84 - protein, 568 aa, synthetic construct; identical to SEQ ID NO: 52
  REGION 1..568: note = Synthetic Polypeptide

SEQ ID NO: 85 - protein, 354 aa, synthetic construct; identical to SEQ ID NO: 30
  REGION 1..354: note = Synthetic Polypeptide

SEQ ID NO: 86 - protein, 568 aa, synthetic construct; identical to SEQ ID NO: 54
  REGION 1..568: note = Synthetic Polypeptide

SEQ ID NO: 87 - protein, 354 aa, synthetic construct; identical to SEQ ID NO: 30
  REGION 1..354: note = Synthetic Polypeptide

SEQ ID NO: 88 - protein, 571 aa, synthetic construct; identical to SEQ ID NO: 60
  REGION 1..571: note = Synthetic Polypeptide

SEQ ID NO: 89 - protein, 354 aa, synthetic construct; identical to SEQ ID NO: 34
  REGION 1..354: note = Synthetic Polypeptide

SEQ ID NO: 90 - protein, 571 aa, synthetic construct; identical to SEQ ID NO: 62
  REGION 1..571: note = Synthetic Polypeptide

SEQ ID NO: 91 - protein, 354 aa, synthetic construct; identical to SEQ ID NO: 34
  REGION 1..354: note = Synthetic Polypeptide

SEQ ID NO: 92 - protein, 568 aa, synthetic construct; identical to SEQ ID NO: 64
  REGION 1..568: note = Synthetic Polypeptide

SEQ ID NO: 93 - protein, 354 aa, synthetic construct; identical to SEQ ID NO: 34
  REGION 1..354: note = Synthetic Polypeptide

SEQ ID NO: 94 - protein, 568 aa, synthetic construct; identical to SEQ ID NO: 66
  REGION 1..568: note = Synthetic Polypeptide

SEQ ID NO: 95 - protein, 354 aa, synthetic construct; identical to SEQ ID NO: 34
  REGION 1..354: note = Synthetic Polypeptide

SEQ ID NO: 96 - protein, 576 aa, synthetic construct
  REGION 1..576: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGQVQLVESGGGVVQPGRSLRLSCAASGFIFSSYAMHWVRQAPGDGLEWVAFMSYDGSNKKYADSVKGRFTISRDNSKNTLYLQMNSLRAEDTAVYYCARDRGIAAGGNYYYYGMDVWGQGTTVTVSSLGGGSGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 97 - protein, 349 aa, synthetic construct; identical to SEQ ID NO: 22
  REGION 1..349: note = Synthetic Polypeptide

SEQ ID NO: 98 - protein, 576 aa, synthetic construct
  REGION 1..576: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGQVQLVESGGGVVQPGRSLRLSCAASGFIFSSYAMHWVRQAPGDGLEWVAFMSYDGSNKKYADSVKGRFTISRDNSKNTLYLQMNSLRAEDTAVYYCARDRGIAAGGNYYYYGMDVWGQGTTVTVSSLGGGSGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 99 - protein, 349 aa, synthetic construct; identical to SEQ ID NO: 22
  REGION 1..349: note = Synthetic Polypeptide

SEQ ID NO: 100 - protein, 573 aa, synthetic construct
  REGION 1..573: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGQVQLVESGGGVVQPGRSLRLSCAASGFIFSSYAMHWVRQAPGDGLEWVAFMSYDGSNKKYADSVKGRFTISRDNSKNTLYLQMNSLRAEDTAVYYCARDRGIAAGGNYYYYGMDVWGQGTTVTVSSLGGGSGASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 101 - protein, 349 aa, synthetic construct; identical to SEQ ID NO: 22
  REGION 1..349: note = Synthetic Polypeptide

SEQ ID NO: 102 - protein, 573 aa, synthetic construct
  REGION 1..573: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGQVQLVESGGGVVQPGRSLRLSCAASGFIFSSYAMHWVRQAPGDGLEWVAFMSYDGSNKKYADSVKGRFTISRDNSKNTLYLQMNSLRAEDTAVYYCARDRGIAAGGNYYYYGMDVWGQGTTVTVSSLGGGSGASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 103 - protein, 349 aa, synthetic construct; identical to SEQ ID NO: 22
  REGION 1..349: note = Synthetic Polypeptide

SEQ ID NO: 104 - protein, 576 aa, synthetic construct
  REGION 1..576: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGQVQLVESGGGVVQPGRSLRLSCAASGFIFSSYAMHWVRQAPGDGLEWVAFMSYDGSNKKYADSVKGRFTISRDNSKNTLYLQMNSLRAEDTAVYYCARDRGIAAGGNYYYYGMDVWGQGTTVTVSSFNRGESASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 105 - protein, 349 aa, synthetic construct; identical to SEQ ID NO: 24
  REGION 1..349: note = Synthetic Polypeptide

SEQ ID NO: 106 - protein, 576 aa, synthetic construct
  REGION 1..576: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGQVQLVESGGGVVQPGRSLRLSCAASGFIFSSYAMHWVRQAPGDGLEWVAFMSYDGSNKKYADSVKGRFTISRDNSKNTLYLQMNSLRAEDTAVYYCARDRGIAAGGNYYYYGMDVWGQGTTVTVSSFNRGESASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 107 - protein, 349 aa, synthetic construct; identical to SEQ ID NO: 24
  REGION 1..349: note = Synthetic Polypeptide

SEQ ID NO: 108 - protein, 573 aa, synthetic construct
  REGION 1..573: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGQVQLVESGGGVVQPGRSLRLSCAASGFIFSSYAMHWVRQAPGDGLEWVAFMSYDGSNKKYADSVKGRFTISRDNSKNTLYLQMNSLRAEDTAVYYCARDRGIAAGGNYYYYGMDVWGQGTTVTVSSFNRGESASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 109 - protein, 349 aa, synthetic construct; identical to SEQ ID NO: 24
  REGION 1..349: note = Synthetic Polypeptide

SEQ ID NO: 110 - protein, 573 aa, synthetic construct
  REGION 1..573: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGQVQLVESGGGVVQPGRSLRLSCAASGFIFSSYAMHWVRQAPGDGLEWVAFMSYDGSNKKYADSVKGRFTISRDNSKNTLYLQMNSLRAEDTAVYYCARDRGIAAGGNYYYYGMDVWGQGTTVTVSSFNRGESASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 111 - protein, 349 aa, synthetic construct; identical to SEQ ID NO: 24
  REGION 1..349: note = Synthetic Polypeptide

SEQ ID NO: 112 - protein, 571 aa, synthetic construct; identical to SEQ ID NO: 80
  REGION 1..571: note = Synthetic Polypeptide

SEQ ID NO: 113 - protein, 354 aa, synthetic construct; identical to SEQ ID NO: 26
  REGION 1..354: note = Synthetic Polypeptide

SEQ ID NO: 114 - protein, 570 aa, synthetic construct
  REGION 1..570: note = Synthetic Polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKGGGSGGGGQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSLGGGSGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 115 - protein, 349 aa, synthetic construct; identical to SEQ ID NO: 14
  REGION 1..349: note = Synthetic Polypeptide

SEQ ID NO: 116 - protein, 567 aa, synthetic construct
  REGION 1..567: note = Synthetic Polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKGGGSGGGGQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSLGGGSGASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 117 - protein, 349 aa, synthetic construct; identical to SEQ ID NO: 14
  REGION 1..349: note = Synthetic Polypeptide

SEQ ID NO: 118 - protein, 567 aa, synthetic construct
  REGION 1..567: note = Synthetic Polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKGGGSGGGGQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSLGGGSGASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 119 - protein, 349 aa, synthetic construct; identical to SEQ ID NO: 14
  REGION 1..349: note = Synthetic Polypeptide

SEQ ID NO: 120 - protein, 570 aa, synthetic construct
  REGION 1..570: note = Synthetic Polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKGGGSGGGGQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSFNRGESASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 121 - protein, 349 aa, synthetic construct; identical to SEQ ID NO: 16
  REGION 1..349: note = Synthetic Polypeptide

SEQ ID NO: 122 - protein, 570 aa, synthetic construct
  REGION 1..570: note = Synthetic Polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKGGGSGGGGQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSFNRGESASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 123 - protein, 349 aa, synthetic construct; identical to SEQ ID NO: 16
  REGION 1..349: note = Synthetic Polypeptide

SEQ ID NO: 124 - protein, 567 aa, synthetic construct
  REGION 1..567: note = Synthetic Polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKGGGSGGGGQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSFNRGESASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 125 - protein, 349 aa, synthetic construct; identical to SEQ ID NO: 16
  REGION 1..349: note = Synthetic Polypeptide

SEQ ID NO: 126 - protein, 567 aa, synthetic construct
  REGION 1..567: note = Synthetic Polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKGGGSGGGGQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSFNRGESASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 127 - protein, 349 aa, synthetic construct; identical to SEQ ID NO: 16
  REGION 1..349: note = Synthetic Polypeptide

SEQ ID NO: 128 - protein, 571 aa, synthetic construct
  REGION 1..571: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSLGGGSGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 129 - protein, 348 aa, synthetic construct; identical to SEQ ID NO: 18
  REGION 1..348: note = Synthetic Polypeptide

SEQ ID NO: 130 - protein, 571 aa, synthetic construct
  REGION 1..571: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSLGGGSGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 131 - protein, 348 aa, synthetic construct; identical to SEQ ID NO: 18
  REGION 1..348: note = Synthetic Polypeptide

SEQ ID NO: 132 - protein, 568 aa, synthetic construct
  REGION 1..568: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSLGGGSGASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 133 - protein, 348 aa, synthetic construct; identical to SEQ ID NO: 18
  REGION 1..348: note = Synthetic Polypeptide

SEQ ID NO: 134 - protein, 568 aa, synthetic construct
  REGION 1..568: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSLGGGSGASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 135 - protein, 348 aa, synthetic construct; identical to SEQ ID NO: 18
  REGION 1..348: note = Synthetic Polypeptide

SEQ ID NO: 136 - protein, 571 aa, synthetic construct; identical to SEQ ID NO: 82
  REGION 1..571: note = Synthetic Polypeptide

SEQ ID NO: 137 - protein, 354 aa, synthetic construct; identical to SEQ ID NO: 26
  REGION 1..354: note = Synthetic Polypeptide

SEQ ID NO: 138 - protein, 571 aa, synthetic construct
  REGION 1..571: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSFNRGESASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 139 - protein, 348 aa, synthetic construct; identical to SEQ ID NO: 20
  REGION 1..348: note = Synthetic Polypeptide

SEQ ID NO: 140 - protein, 568 aa, synthetic construct
  REGION 1..568: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSFNRGESASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 141 - protein, 348 aa, synthetic construct; identical to SEQ ID NO: 20
  REGION 1..348: note = Synthetic Polypeptide

SEQ ID NO: 142 - protein, 568 aa, synthetic construct
  REGION 1..568: note = Synthetic Polypeptide
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKGGGSGGGGEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSFNRGESASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 143 - protein, 348 aa, synthetic construct; identical to SEQ ID NO: 20
  REGION 1..348: note = Synthetic Polypeptide

SEQ ID NO: 144 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 145 - protein, 189 aa, Homo sapiens
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 146 - protein, 5 aa, synthetic construct
  REGION 1..5: note = Synthetic Polypeptide
DYAMH

SEQ ID NO: 147 - protein, 17 aa, synthetic construct
  REGION 1..17: note = Synthetic Polypeptide
AITWNSGHIDYADSVEG

SEQ ID NO: 148 - protein, 12 aa, synthetic construct
  REGION 1..12: note = Synthetic Polypeptide
VSYLSTASSLDY

SEQ ID NO: 149 - protein, 11 aa, synthetic construct
  REGION 1..11: note = Synthetic Polypeptide
RASQGIRNYLA

SEQ ID NO: 150 - protein, 7 aa, synthetic construct
  REGION 1..7: note = Synthetic Polypeptide
AASTLQS

SEQ ID NO: 151 - protein, 9 aa, synthetic construct
  REGION 1..9: note = Synthetic Polypeptide
QRYNRAPYT

SEQ ID NO: 152 - protein, 5 aa, synthetic construct
  REGION 1..5: note = Synthetic Polypeptide
SYAMH

SEQ ID NO: 153 - protein, 17 aa, synthetic construct
  REGION 1..17: note = Synthetic Polypeptide
FMSYDGSNKKYADSVKG

SEQ ID NO: 154 - protein, 9 aa, synthetic construct
  REGION 1..9: note = Synthetic Polypeptide
NYYYYGMDV

SEQ ID NO: 155 - protein, 11 aa, synthetic construct
  REGION 1..11: note = Synthetic Polypeptide
RASQSVYSYLA

SEQ ID NO: 156 - protein, 7 aa, synthetic construct
  REGION 1..7: note = Synthetic Polypeptide
DASNRAT

SEQ ID NO: 157 - protein, 10 aa, synthetic construct
  REGION 1..10: note = Synthetic Polypeptide
QQRSNWPPFT

SEQ ID NO: 158 - protein, 5 aa, synthetic construct
  REGION 1..5: note = Synthetic Polypeptide
DQTIH

SEQ ID NO: 159 - protein, 17 aa, synthetic construct
  REGION 1..17: note = Synthetic Polypeptide
YIYPRDDSPKYNENFKG

SEQ ID NO: 160 - protein, 11 aa, synthetic construct
  REGION 1..11: note = Synthetic Polypeptide
PDRSGYAWFIY

SEQ ID NO: 161 - protein, 11 aa, synthetic construct
  REGION 1..11: note = Synthetic Polypeptide
KASRDVAIAVA

SEQ ID NO: 162 - protein, 7 aa, synthetic construct
  REGION 1..7: note = Synthetic Polypeptide
WASTRHT

SEQ ID NO: 163 - protein, 9 aa, synthetic construct
  REGION 1..9: note = Synthetic Polypeptide
HQYSSYPFT